(19) United States
(12) Patent Application Publication
(10) Pub. No.: US 2025/0011811 A1
(43) Pub. Date: Jan. 9, 2025

(54) STABLE PRODUCTION SYSTEMS FOR AAV VECTOR PRODUCTION

(71) Applicant: ASIMOV INC., Boston, MA (US)

(72) Inventors: Christopher S. Stach, Boston, MA (US); Alec A.K. Nielsen, Brooklne, MA (US)

(73) Assignee: ASIMOV INC., Boston, MA (US)

(21) Appl. No.: 18/708,263

(22) PCT Filed: Nov. 9, 2022
(86) PCT No.: PCT/US2022/079543

§ 371 Date: May 8, 2024

Related U.S. Application Data
(60) Provisional application No. 63/277,335, filed on Nov. 9, 2021.

(51) Int. Cl. C12N 15/86

(52) U.S. Cl. CPC C12N 15/86; C12N 2750/14143; C12N 2750/14152; C12N 2830/003

(57) ABSTRACT

Described herein are AAV vector production systems and HSV-helper systems. Also described herein are engineered cells and kits comprising an AAV vector production system and/or an HSV-helper system and methods of using the same for AAV vector production.

### RELATED APPLICATIONS

This application claims the benefit under 35 U.S.C. § 119 of U.S. provisional application Ser. No. 63/277,335, filed Nov. 9, 2021, the entire contents of which are incorporated by reference herein.

### FIELD

Described herein are AAV vector production systems and HSV-helper systems. Also described herein are engineered cells and kits comprising an AAV vector production system and/or an HSV-helper system and methods of using the same for AAV vector production.

### REFERENCE TO AN ELECTRONIC SEQUENCE LISTING

The contents of the electronic sequence listing (A121070006WO00-SEQ-ARM.xml; Size: 82,612 bytes; and Date of Creation: Nov. 4, 2022) is herein incorporated by reference in its entirety.

### BACKGROUND

Viral vectors are a promising gene delivery modality for cell and gene therapy. Viral vectors can be modified to carry therapeutic genetic payloads to cells within a subject. The production of viral vectors normally entails transient transfection of plasmids containing genes required for viral vector production into cell culture. However, transient transfection has several shortfalls. Large quantities of DNA and transfection reagent must be procured for the transfection process, which is costly. Also, poor transfection efficiency can result in minimal numbers of “transfected” cells and increased variation associated with transfection steps and viral production.

### SUMMARY

Described herein are AAV vector production systems and HSV-helper systems. Also described herein are kits comprising an AAV vector production system and/or an HSV-helper system. Finally, engineered cells comprising an AAV vector production system (or at least a portion thereof) and/or an HSV-helper system are described, as well as methods of using the engineered cells for AAV vector production.

In some aspects, the disclosure relates to engineered cells for adeno-associated virus (AAV) production. In some embodiments, an engineered cell comprises one or more stably integrated polynucleic acids collectively comprising a nucleic acid sequence encoding for each of: Rep52 or Rep40; Rep78 or Rep68; E2A; E4Orf6; VARNA; VP1; VP2; VP3; and AAP; each of which is operably linked to a chemically inducible promoter.

In some embodiments, the engineered cell comprises a chemically inducible promoter comprising the nucleic acid sequence of one or more of SEQ ID NOs: 1-9 operably linked to a nucleic acid sequence encoding for at least one of Rep52, Rep40, Rep78, Rep68, E2A, E4Orf6, VARNA, VP1, VP2, VP3, and AAP.

In some embodiments, the engineered cell comprises at least two chemically inducible promoters, wherein two or more of the at least two chemically inducible promoters comprise the same nucleic acid sequence. In some embodiments, each of the at least two chemically inducible promoters comprise the same nucleic acid sequence.

In some embodiments, the engineered cell comprises a nucleic acid sequence encoding for Rep52 operably linked to a chemically inducible promoter comprising the nucleic acid sequence of one or more of SEQ ID NOs: 1-9, optionally wherein Rep52 comprises the amino acid sequence of SEQ ID NO: 18.

In some embodiments, the engineered cell comprises a nucleic acid sequence encoding for Rep40 operably linked to a chemically inducible promoter comprising the nucleic acid sequence of one or more of SEQ ID NOs: 1-9, optionally wherein Rep40 comprises the amino acid sequence of SEQ ID NO: 20.

In some embodiments, the engineered cell comprises a nucleic acid sequence encoding for Rep78 operably linked to a chemically inducible promoter comprising the nucleic acid sequence of one or more of SEQ ID NOs: 1-9, optionally wherein Rep78 comprises the amino acid sequence of any one of SEQ ID NOs: 21 or 22.

In some embodiments, the engineered cell comprises a nucleic acid sequence encoding for Rep68 operably linked to a chemically inducible promoter comprising the nucleic acid sequence of one or more of SEQ ID NOs: 1-9, optionally wherein Rep68 comprises the amino acid sequence of any one of SEQ ID NOs: 26 or 27.

In some embodiments, the engineered cell comprises a nucleic acid sequence encoding for E2A operably linked to a chemically inducible promoter comprising the nucleic acid sequence of one or more of SEQ ID NOs: 1-9, optionally wherein E2A comprises the amino acid sequence of SEQ ID NO: 29.

In some embodiments, the engineered cell comprises a nucleic acid sequence encoding for E4Orf6 operably linked to a chemically inducible promoter comprising the nucleic acid sequence of one or more of SEQ ID NOs: 1-9, optionally wherein E4Orf6 comprises the amino acid sequence of SEQ ID NO: 35.

In some embodiments, the engineered cell comprises a nucleic acid sequence encoding for VARNA operably linked to a chemically inducible promoter comprising the nucleic acid sequence of one or more of SEQ ID NOs: 1-9, optionally wherein VARNA comprises the nucleic acid sequence of SEQ ID NO: 38.

In some embodiments, the engineered cell comprises a nucleic acid sequence encoding for VP1 operably linked to a chemically inducible promoter comprising the nucleic acid sequence of one or more of SEQ ID NOs: 1-9, optionally wherein VP1 comprises the amino acid sequence of any one of SEQ ID NOs: 30 or 31.

In some embodiments, the engineered cell comprises a nucleic acid sequence encoding for VP2 operably linked to a chemically inducible promoter comprising the nucleic acid sequence of one or more of SEQ ID NOs: 1-9, optionally wherein VP2 comprises the amino acid sequence of SEQ ID NO: 33.

In some embodiments, the engineered cell comprises a nucleic acid sequence encoding for VP3 operably linked to a chemically inducible promoter comprising the nucleic acid sequence of one or more of SEQ ID NOs: 1-9, optionally wherein VP3 comprises the amino acid sequence of SEQ ID NO: 34.

In some embodiments, the engineered cell comprises a nucleic acid sequence encoding for AAP operably linked to a chemically inducible promoter comprising the nucleic acid sequence of one or more of SEQ ID NOs: 1-9, optionally wherein AAP comprises the amino acid sequence of SEQ ID NO: 37.

In some embodiments, the engineered cell comprises a first stably integrated polynucleic acid, wherein the first stably integrated polynucleic acid comprises the nucleic acid sequence encoding for Rep52 or Rep40 and the nucleic acid sequence encoding for Rep78 or Rep68. In some embodiments, the nucleic acid sequence encoding for Rep52 or Rep40 and the nucleic acid sequence encoding for Rep78 or Rep68 are each operably linked to a first chemically inducible promoter. In some embodiments, the first chemically inducible promoter comprises the nucleic acid sequence of one or more of SEQ ID NOs: 1-9. In some embodiments, the first stably integrated polynucleic acid further comprises a nucleic acid sequence encoding for an internal ribosome entry site (IRES). In some embodiments, the nucleic acid sequence encoding for the IRES separates the nucleic acid sequence encoding for Rep52 or Rep40 and the nucleic acid sequence encoding for Rep78 or Rep68. In some embodiments, the first stably integrated polynucleic acid further comprises a selection marker that is operably linked to a promoter.

In some embodiments, the engineered cell comprises a second stably integrated polynucleic acid, wherein the second stably integrated polynucleic acid comprises the nucleic acid sequences encoding for E2A, E4Orf6, and VARNA. In some embodiments, the nucleic acid sequences encoding for E2A, E4Orf6, and VARNA are each operably linked to a second chemically inducible promoter. In some embodiments, the second chemically inducible promoter comprises the nucleic acid sequence of one or more of SEQ ID NOs: 1-9. In some embodiments, the second stably integrated polynucleic acid further comprises a nucleic acid sequence encoding an IRES. In some embodiments, the nucleic acid sequence encoding for the IRES separates the nucleic acid sequence encoding for E2A and the nucleic acid sequence encoding for E4Orf6. In some embodiments, the second stably integrated polynucleic acid further comprises a selection marker that is operably linked to a promoter.

In some embodiments, the engineered cell comprises a third stably integrated polynucleic acid, wherein the third stably integrated polynucleic acid comprises the nucleic acid sequences encoding for VP1, VP2, VP3, and AAP. In some embodiments, the third stably integrated polynucleic acid further comprises a selection marker that is operably linked to a promoter. In some embodiments, the nucleic acid sequences encoding for VP1, VP2, VP3, and AAP are each operably linked to a third chemically inducible promoter. In some embodiments, the third chemically inducible promoter comprises the nucleic acid sequence of one or more of SEQ ID NOs: 1-9.

In some embodiments, the engineered cell comprises a fourth stably integrated polynucleic acid, wherein the fourth stably integrated polynucleic acid comprises a nucleic acid sequence encoding for a transcriptional activator that, when expressed in the presence of a small molecule inducer, binds to a chemically inducible promoter of the engineered cell. In some embodiments, the transcriptional activator comprises the amino acid sequence of any one of SEQ ID NOs: 10-16. In some embodiments, the fourth stably integrated polynucleic acid further comprises a selection marker that is operably linked to a promoter.

In some embodiments, the engineered cell further comprises a stable landing pad.

In some embodiments, the engineered cell is derived from a HEK293 cell, a HeLa cell, a BHK cell, or a Sf9 cell.

In some aspect, the disclosure relates to engineered cells for AAV production. In some embodiments, an engineered cell for AAV production comprises one or more stably integrated polynucleic acid collectively comprising a nucleic acid sequence encoding for each of: Rep52 or Rep40; Rep78 or Rep68; E2A; E4Orf6; VARNA; VP1; VP2; VP3; and AAP; at least one of which is operably linked to a chemically inducible promoter; wherein: (i) the nucleic acid sequence encoding for Rep68 lacks the Rep52 start codon; (ii) the nucleic acid sequence encoding for Rep78 lacks the Rep52 start codon and the Rep68/40 splice site; (iii) the nucleic acid sequence encoding for Rep52 lacks the Rep40 splice site; and (iv) the nucleic acid sequence encoding for VP1 lacks start codons for VP2, VP3, and AAP.

In some embodiments, the engineered cell comprises a chemically inducible promoter comprising the nucleic acid sequence of one or more of SEQ ID NOs: 1-9, operably linked to a nucleic acid sequence encoding for at least one of Rep52, Rep40, Rep78, Rep68, E2A, E4Orf6, VARNA, SC-VP1, VP2, VP3, and AAP.

In some embodiments, the engineered cell comprises at least two chemically inducible promoters, wherein two or more of the at least two chemically inducible promoters comprise the same nucleic acid sequence. In some embodiments, each of the at least two chemically inducible promoters comprise the same nucleic acid sequence.

In some embodiments, the engineered cell comprises a nucleic acid sequence encoding for Rep52 operably linked to a chemically inducible promoter comprising the nucleic acid sequence of one or more of SEQ ID NOs: 1-9, optionally wherein Rep52 comprises the amino acid sequence of SEQ ID NO: 18, optionally wherein the nucleic acid sequence encoding for Rep52 comprises the nucleic acid sequence of SEQ ID NO: 19.

In some embodiments, the engineered cell comprises a nucleic acid sequence encoding for Rep40 operably linked to a chemically inducible promoter comprising the nucleic acid sequence of one or more of SEQ ID NOs: 1-9, optionally wherein Rep40 comprises the amino acid sequence of SEQ ID NO: 20.

In some embodiments, the engineered cell comprises a nucleic acid sequence encoding for Rep78 operably linked to a chemically inducible promoter comprising the nucleic acid sequence of one or more of SEQ ID NOs: 1-9, optionally wherein Rep78 comprises the amino acid sequence of SEQ ID NO: 22, optionally wherein the nucleic acid sequence encoding for Rep78 comprises the nucleic acid sequence of SEQ ID NO: 23.

In some embodiments, the engineered cell comprises a nucleic acid sequence encoding for Rep68 operably linked to a chemically inducible promoter comprising the nucleic acid sequence of one or more of SEQ ID NOs: 1-9, optionally wherein Rep68 comprises the amino acid sequence of SEQ ID NO: 27, optionally wherein the nucleic acid sequence encoding for Rep68 comprises the nucleic acid sequence of SEQ ID NO: 28.

In some embodiments, the engineered cell comprises a nucleic acid sequence encoding for E2A operably linked to a chemically inducible promoter comprising the nucleic acid sequence of one or more of SEQ ID NOs: 1-9, optionally wherein E2A comprises the amino acid sequence of SEQ ID NO: 31.

In some embodiments, the engineered cell comprises a nucleic acid sequence encoding for E4Orf6 operably linked to a chemically inducible promoter comprising the nucleic acid sequence of one or more of SEQ ID NOs: 1-9, optionally wherein E4Orf6 comprises the amino acid sequence of SEQ ID NO: 35, optionally wherein the nucleic acid sequence encoding for E4Orf6 comprises the nucleic acid sequence of SEQ ID NO: 36.

In some embodiments, the engineered cell comprises a nucleic acid sequence encoding for VARNA operably linked to a chemically inducible promoter comprising the nucleic acid sequence of one or more of SEQ ID NOs: 1-9, optionally wherein VARNA comprises the nucleic acid sequence of SEQ ID NO: 38.

In some embodiments, the engineered cell comprises a nucleic acid sequence encoding for VP1 operably linked to a chemically inducible promoter comprising the nucleic acid sequence of one or more of SEQ ID NOs: 1-9, optionally wherein VP1 comprises the amino acid sequence of SEQ ID NO: 31, optionally wherein the nucleic acid sequence encoding for VP1 comprises the nucleic acid sequence of SEQ ID NO: 32.

In some embodiments, the engineered cell comprises a nucleic acid sequence encoding for VP2 operably linked to a chemically inducible promoter comprising the nucleic acid sequence of one or more of SEQ ID NOs: 1-9, optionally wherein VP2 comprises the amino acid sequence of SEQ ID NO: 33.

In some embodiments, the engineered cell comprises a nucleic acid sequence encoding for VP3 operably linked to a chemically inducible promoter comprising the nucleic acid sequence of one or more of SEQ ID NOs: 1-9, optionally wherein VP3 comprises the amino acid sequence of SEQ ID NO: 35.

In some embodiments, the engineered cell comprises a nucleic acid sequence encoding for AAP operably linked to a chemically inducible promoter comprising the nucleic acid sequence of one or more of SEQ ID NOs: 1-9, optionally wherein AAP comprises the amino acid sequence of SEQ ID NO: 37.

In some embodiments, the engineered cell comprises a first stably integrated polynucleic acid, wherein the first stably integrated polynucleic acid comprises the nucleic acid sequence encoding for Rep52 or Rep40 and the nucleic acid sequence encoding for Rep78 or Rep68. In some embodiments, the first stably integrated polynucleic acid comprising the nucleic acid sequence encoding for Rep52 and the nucleic acid sequence encoding for Rep78 or Rep68. In some embodiments, the nucleic acid sequence encoding for Rep52 and the nucleic acid sequence encoding for Rep78 or Rep68 are each operably linked to a first chemically inducible promoter. In some embodiments, the first chemically inducible promoter comprises the nucleic acid sequence of one or more of SEQ ID NOs: 1-9. In some embodiments, the first stably integrated polynucleic acid further comprises a nucleic acid sequence encoding for an internal ribosome entry site (IRES) or a 2A peptide. In some embodiments, the nucleic acid sequence encoding for the IRES or the 2A peptide separates the nucleic acid sequence encoding for Rep52 and the nucleic acid sequence encoding for Rep78 or Rep68. In some embodiments, the first stably integrated polynucleic acid further comprises a selection marker that is operably linked to a promoter.

In some embodiments, the engineered cell comprises a second stably integrated polynucleic acid, wherein the second stably integrated polynucleic acid comprises the nucleic acid sequence encoding for E2A, the nucleic acid sequence encoding for E4Orf6, and the nucleic acid sequence encoding for VARNA. In some embodiments, the nucleic acid sequence encoding for E2A, the nucleic acid sequence encoding for E4Orf6, and the nucleic acid sequence encoding for VARNA are each operably linked to the second chemically inducible promoter. In some embodiments, the second chemically inducible promoter comprises the nucleic acid sequence of one or more of SEQ ID NOs: 1-9. In some embodiments, the second stably integrated polynucleic acid further comprises a nucleic acid sequence encoding for an internal ribosome entry site (IRES) or a 2A peptide. In some embodiments, the nucleic acid sequence encoding for the IRES or the 2A peptide separates the nucleic acid sequence encoding for E2A and the nucleic acid sequence encoding E4Orf6. In some embodiments, the second stably integrated polynucleic acid further comprises a selection marker that is operably linked to a promoter.

In some embodiments, the engineered cell comprises a third stably integrated polynucleic acid, wherein the third stably integrated polynucleic acid comprises the nucleic acid sequence encoding for VP1, the nucleic acid sequence encoding for VP2, the nucleic acid sequence encoding for VP3, and the nucleic acid sequence encoding for AAP. In some embodiments, the nucleic acid sequence encoding for VP1 is operably linked to a third chemically inducible promoter and the nucleic acid sequence encoding for VP2, the nucleic acid sequence encoding for VP3, and the nucleic acid sequence encoding for AAP are each operably linked to a fourth chemically inducible promoter. In some embodiments, the third chemically inducible promoter comprises the nucleic acid sequence of one or more of SEQ ID NOs: 1-9. In some embodiments, the fourth chemically inducible promoter comprises the nucleic acid sequence of one or more of SEQ ID NOs: 1-9. In some embodiments, the third stably integrated polynucleic acid further comprises a selection marker that is operably linked to a promoter.

In some embodiments, the engineered cell comprises a fourth stably integrated polynucleic acid, wherein the fourth stably integrated polynucleic acid comprises a nucleic acid sequence encoding for a transcriptional activator that, when expressed in the presence of a small molecule inducer, binds to a chemically inducible promoter of the engineered cell. In some embodiments, the transcriptional activator comprises the amino acid sequence of any one of SEQ ID NOs: 10-16. In some embodiments, the fourth stably integrated polynucleic molecule further comprises a selection marker that is operably linked to a promoter.

In some embodiments, the engineered cell comprises one or more stably integrated polynucleic acid collectively comprising a nucleic acid sequence encoding for each of: UL5; UL8; UL29; UL30; UL42; and UL52; each of which is operably linked to a chemically inducible promoter.

In some embodiments, the one or more stably integrated polynucleic acid further comprise a nucleic acid sequence encoding for one or more of UL12, ICP0, ICP4, and ICP22. In some embodiments, the one or more stably integrated polynucleic acid further comprise a nucleic acid sequence encoding for each of UL12, ICP0, ICP4, and ICP22.

In some embodiments, the engineered cell comprises a chemically inducible promoter comprising the nucleic acid sequence of one or more of SEQ ID NOs: 1-9 operably linked to a nucleic acid sequence encoding for at least one of UL5, UL8, UL29, UL30, UL42, UL52, UL12, ICP0, ICP4, and ICP22.

In some embodiments, the engineered cell comprises at least two chemically inducible promoters, wherein two or more of the at least two chemically inducible promoters comprise the same nucleic acid sequence. In some embodiments, each of the at least two chemically inducible promoters comprise the same nucleic acid sequence.

In some embodiments, the engineered cell comprises a nucleic acid sequence encoding for UL5 operably linked to a chemically inducible promoter comprising the nucleic acid sequence of one or more of SEQ ID NOs: 1-9, optionally wherein UL5 comprises the amino acid sequence of SEQ ID NO: 41.

In some embodiments, the engineered cell comprises a nucleic acid sequence encoding for UL8 operably linked to a chemically inducible promoter comprising the nucleic acid sequence of one or more of SEQ ID NOs: 1-9, optionally wherein UL8 comprises the amino acid sequence of SEQ ID NO: 42.

In some embodiments, the engineered cell comprises a nucleic acid sequence encoding for UL29 operably linked to a chemically inducible promoter comprising the nucleic acid sequence of one or more of SEQ ID NOs: 1-9, optionally wherein UL29 comprises the amino acid sequence of SEQ ID NO: 44.

In some embodiments, the engineered cell comprises a nucleic acid sequence encoding for UL30 operably linked to a chemically inducible promoter comprising the nucleic acid sequence of one or more of SEQ ID NOs: 1-9, optionally wherein UL30 comprises the amino acid sequence of SEQ ID NO: 39.

In some embodiments, the engineered cell comprises a nucleic acid sequence encoding for UL42 operably linked to a chemically inducible promoter comprising the nucleic acid sequence of one or more of SEQ ID NOs: 1-9, optionally wherein UL42 comprises the amino acid sequence of SEQ ID NO: 40.

In some embodiments, the engineered cell comprises a nucleic acid sequence encoding for UL52 operably linked to a chemically inducible promoter comprising the nucleic acid sequence of one or more of SEQ ID NOs: 1-9, optionally wherein UL52 comprises the amino acid sequence of SEQ ID NO: 43.

In some embodiments, the engineered cell comprises a nucleic acid sequence encoding for UL12 operably linked to a chemically inducible promoter comprising the nucleic acid sequence of one or more of SEQ ID NOs: 1-9, optionally wherein UL12 comprises the amino acid sequence of SEQ ID NO: 50.

In some embodiments, the engineered cell comprises a nucleic acid sequence encoding for ICP0 operably linked to a chemically inducible promoter comprising the nucleic acid sequence of one or more of SEQ ID NOs: 1-9, optionally wherein ICP0 comprises the amino acid sequence of SEQ ID NO: 51.

In some embodiments, the engineered cell comprises a nucleic acid sequence encoding for ICP4 operably linked to a chemically inducible promoter comprising the nucleic acid sequence of one or more of SEQ ID NOs: 1-9, optionally wherein ICP4 comprises the amino acid sequence of SEQ ID NO: 52.

In some embodiments, the engineered cell comprises a nucleic acid sequence encoding for ICP22 operably linked to a chemically inducible promoter comprising the nucleic acid sequence of one or more of SEQ ID NOs: 1-9, optionally wherein ICP22 comprises the amino acid sequence of SEQ ID NO: 53.

In some embodiments, the engineered cell comprises a first stably integrated polynucleic acid, wherein the first stably integrated polynucleic acid comprises the nucleic acid sequence encoding for UL30 and the nucleic acid sequence encoding for UL42. In some embodiments, the nucleic acid sequence encoding for UL30 and the nucleic acid sequence encoding for UL42 are each operably linked to a first chemically inducible promoter. In some embodiments, the first chemically inducible promoter comprises the nucleic acid sequence of one or more of SEQ ID NOs: 1-9. In some embodiments, the first stably integrated polynucleic acid further comprises a nucleic acid sequence encoding for an internal ribosome entry site (IRES). In some embodiments, the nucleic acid sequence encoding for the IRES separates the nucleic acid sequence encoding for UL30 and the nucleic acid sequence encoding for UL42. In some embodiments, the first stably integrated polynucleic acid further comprises a selection marker that is operably linked to a promoter.

In some embodiments, the engineered cell comprises a second stably integrated polynucleic acid, wherein the second stably integrated polynucleic acid comprises the nucleic acid sequence encoding for UL5, the nucleic acid sequence encoding for UL8, the nucleic acid sequence encoding for UL52, and the nucleic acid sequence encoding for UL29. In some embodiments, the nucleic acid sequence encoding for UL5, the nucleic acid sequence encoding for UL8, and the nucleic acid sequence encoding for UL52 are each operably linked to a second chemically inducible promoter, and the nucleic acid sequence encoding for UL29 is operably linked to a third chemically inducible promoter. In some embodiments, the second chemically inducible promoter comprises the nucleic acid sequence of one or more of SEQ ID NOs: 1-9. In some embodiments, the third chemically inducible promoter the nucleic acid sequence of one or more of SEQ ID NOs: 1-9. In some embodiments, the second stably integrated polynucleic acid further comprises a nucleic acid sequence encoding for an internal ribosome entry site (IRES). In some embodiments, the nucleic acid sequence encoding for the IRES separates the nucleic acid sequence encoding for UL8 and the nucleic acid sequence encoding UL52. In some embodiments, the second stably integrated polynucleic acid further comprises a selection marker that is operably linked to a promoter.

In some embodiments, the engineered cell comprises a third stably integrated polynucleic acid, wherein the third stably integrated polynucleic acid comprises a nucleic acid sequence encoding for a transcriptional activator that, when expressed in the presence of a small molecule inducer, binds to a chemically inducible promoter of the engineered cell. In some embodiments, the transcriptional activator comprises the amino acid sequence of any one of SEQ ID NOs: 10-16. In some embodiments, the third stably integrated polynucleic acid further comprises a selection marker that is operably linked to a promoter.

In some embodiments, the engineered cell comprises a fourth stably integrated polynucleic acid, wherein the fourth stably integrated polynucleic acid comprises the nucleic acid sequence encoding for ICP0, the nucleic acid sequence encoding for UL12, the nucleic acid sequence encoding for ICP4, and the nucleic acid sequence encoding for ICP22.

In some embodiments, the nucleic acid sequence encoding for ICP0, the nucleic acid sequence encoding for UL12, and the nucleic acid sequence encoding for ICP4, are each linked to a fourth chemically inducible promoter and the nucleic acid sequence encoding for ICP22 is operably linked to a fifth chemically inducible promoter.

In some embodiments, the fourth chemically inducible promoter and fifth chemically inducible promoter comprises the nucleic acid sequence of one or more of SEQ ID NOs: 1-9.

In some embodiments, the first stably integrated polynucleic acid further comprises a nucleic acid sequence encoding for an internal ribosome entry site (IRES).

In some embodiments, the nucleic acid sequence encoding for the IRES separates the nucleic acid sequence encoding for UL12 and the nucleic acid sequence encoding for ICP4.

In some embodiments, the first stably integrated polynucleic acid further comprises a selection marker that is operably linked to a promoter.

In some embodiments, the engineered cell further comprises a stable landing pad.

In some embodiments, the engineered cell is derived from a HEK293 cell, a HeLa cell, a BHK cell, or a Sf9 cell.

In some aspects, the disclosure relates to kits comprising an engineered cell described herein.

In some embodiments, a kit further comprises a transfer polynucleic acid molecule comprising, from 5′ to 3′: (i) a nucleic acid sequence of a 5′ AAV inverted tandem repeat (ITR); (ii) a multiple cloning site; and (iii) a nucleic acid sequence of a 3′ AAV inverted tandem repeat (ITR). In some embodiments, the transfer polynucleic acid is a plasmid or a vector.

In some embodiments, the kit further comprises a small molecule inducer corresponding to a chemically inducible promoter of the engineered cell.

In some embodiments, the engineered cell of the kit comprises a chemically inducible promoter comprising the nucleic acid sequence of one or more of SEQ ID NOs: 1-9 operably linked to the nucleic acid sequence encoding for at least one of Rep52, Rep40, Rep78, Rep68, E2A, E4Orf6, VARNA, VP1, VP2, VP3, and AAP. In some embodiments, the nucleic acid sequence encoding for Rep52 or Rep40, the nucleic acid sequence encoding for Rep78 or Rep68, the nucleic acid sequence encoding for E2A, the nucleic acid sequence encoding for EOrf6, the nucleic acid sequence encoding for VARNA, the nucleic acid sequence encoding for VP1, the nucleic acid sequence encoding for VP2, the nucleic acid sequence encoding for Vp3, and the nucleic acid sequence encoding for AAP are each operably linked to a chemically inducible promoter comprising the nucleic acid sequence of one or more of SEQ ID NOs: 1-9.

In some embodiments, the engineered cell of the kit comprises a chemically inducible promoter comprising the nucleic acid sequence of one or more of SEQ ID NOs: 1-9 operably linked to the nucleic acid sequence encoding for at least one of ICP0, ICP4, ICP22, UL5, UL8, UL12, UL29, UL30, UL42, and UL52. In some embodiments, the nucleic acid sequence encoding for ICP0, the nucleic acid sequence encoding for ICP4, the nucleic acid sequence encoding for ICP22, the nucleic acid sequence encoding for UL5, the nucleic acid sequence encoding for UL8, the nucleic acid sequence encoding for UL12, the nucleic acid sequence encoding for UL29, the nucleic acid sequence encoding for UL30, the nucleic acid sequence encoding for UL42, and the nucleic acid sequence encoding for UL52 are each operably linked to a chemically inducible promoter comprising the nucleic acid sequence of one or more of SEQ ID NOs: 1-9.

In some embodiments, the engineered cell of the kit comprises at least two chemically inducible promoters. In some embodiments, two or more of the at least two chemically inducible promoters are distinct.

In some embodiments, the kit comprises a polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the engineered cell, optionally wherein the engineered cell comprises the polynucleic acid comprising the nucleic acid sequence of the transcriptional activator. In some embodiments, the transcriptional activator comprises the amino acid sequence of any one of SEQ ID NOs: 10-12.

In some embodiments, the kit comprises the small molecule inducer doxycycline or tetracycline.

In some aspects, the disclosure relates to methods of producing an AAV vector.

In some embodiments, the method comprises: (a) introducing a transfer polynucleic acid into an engineered cell described herein (e.g., comprising one or more polynucleotides collectively encoding for Rep52 or Rep40; Rep78 or SC-Rep68; E2A; E4Orf6; VARNA; VP1; VP2; VP3; AAP; and a transcriptional activator); and (b) contacting the engineered cell with a small molecule inducer corresponding to a chemically inducible promoter of the engineered cell, thereby inducing expression of: Rep52 or Rep40; Rep78 or SC-Rep68; E2A; E4Orf6; VARNA; VP1; VP2; VP3; and AAP; wherein the engineered cell comprises a heterologous polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the engineered cell; wherein (b) occurs before, concurrently with, or after (a).

In some embodiments, the engineered cell comprises a chemically inducible promoter comprising the nucleic acid sequence of one or more of SEQ ID NOs: 1-9 operably linked to the nucleic acid sequence encoding for at least one of Rep52, Rep40, Rep78, Rep68, E2A, E4Orf6, VARNA, VP1, VP2, VP3, and AAP.

In some embodiments, the nucleic acid sequence encoding for Rep52 or Rep40, the nucleic acid sequence encoding for Rep78 or Rep68, the nucleic acid sequence encoding for E2A, the nucleic acid sequence encoding for EOrf6, the nucleic acid sequence encoding for VARNA, the nucleic acid sequence encoding for VP1, the nucleic acid sequence encoding for VP2, the nucleic acid sequence encoding for VP3, and the nucleic acid sequence encoding for AAP are each operably linked to a chemically inducible promoter comprising the nucleic acid sequence of one or more of SEQ ID NOs: 1-9. In some embodiments, the engineered cell comprises at least two chemically inducible promoters. In some embodiments, two or more of the at least two chemically inducible promoters are distinct.

In some embodiments, the transcriptional activator comprises the amino acid sequence of any one of SEQ ID NOs: 10-12.

In some embodiments, the small molecule inducer is doxycycline or tetracycline.

In some embodiments, the method comprises: (a) introducing a transfer polynucleic acid into the engineered cell of any one of claims F1-F28; and (b) contacting the engineered cell with a small molecule inducer corresponding to a chemically inducible promoter of the engineered cell, thereby inducing expression of: UL5, UL8, UL29, UL30, UL42, and UL52; wherein the engineered cell comprises a heterologous polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the engineered cell; wherein (b) occurs before, concurrently with, or after (a).

In some embodiments, the engineered cell comprises the engineered cell comprises one or more polynucleic acids collectively comprising a nucleic acid sequence encoding for each of: Rep52 or Rep40; Rep78 or Rep68; E2A; E4Orf6; VARNA; VP1; VP2; VP3; and AAP.

In some embodiments, the engineered cell comprises one or more stably integrated polynucleic acids collectively comprising a nucleic acid sequence encoding for each of: Rep52 or Rep40; Rep78 or Rep68; E2A; E4Orf6; VARNA; VP1; VP2; VP3; and AAP.

In some embodiments, the method further comprises: (c) introducing one or more polynucleic acids collectively comprising a nucleic acid sequence encoding for each of: Rep52 or Rep40; Rep78 or Rep68; E2A; E4Orf6; VARNA; VP1; VP2; VP3; and AAP; wherein (c) occurs prior to, concurrently with, or after (a) or (b).

In some embodiments, the engineered cell comprises a chemically inducible promoter comprising the nucleic acid sequence of one or more of SEQ ID NOs: 1-9 operably linked to the nucleic acid sequence encoding for at least one of ICP0, ICP4, ICP22, UL5, UL8, UL12 UL29, UL30, UL42, and UL52.

In some embodiments, the nucleic acid sequence encoding for Rep52 or Rep40, the nucleic acid sequence encoding for ICP0, the nucleic acid sequence encoding for ICP4, the nucleic acid sequence encoding for ICP22, the nucleic acid sequence encoding for UL5, the nucleic acid sequence encoding for UL8, the nucleic acid sequence encoding for IL12, the nucleic acid sequence encoding for UL29, the nucleic acid sequence encoding for UL30, the nucleic acid sequence encoding for UL42, and the nucleic acid sequence encoding for UL52 are each operably linked to a chemically inducible promoter comprising the nucleic acid sequence of one or more of SEQ ID NOs: 1-9.

In some embodiments, the engineered cell comprises at least two chemically inducible promoters. In some embodiments, two or more of the at least two chemically inducible promoters are distinct.

In some embodiments, the transcriptional activator comprises the amino acid sequence of any one of SEQ ID NOs: 10-12.

In some embodiments, the small molecule inducer is doxycycline or tetracycline.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 shows a plasmid schematic for inducible AAV transient transfection plasmids. An EGFP expressing transfer plasmid was used as the payload for AAV. Constitutively expressing Tet-On 3G rtTA and pTRE3G dox-inducible: linked Rep52/40 and Rep78/68, linked E2A and E4orf6 with VARNA cassette, and AAV2 Cap, were used to test the system in a transient transfection using HEK293FT as the host cell line.

FIG. 2 shows a plasmid schematic for inducible AAV stable integration plasmids. The Tet-On 3G plasmid expresses the rtTA required for induction of gene expression from TRE3G promoters. AAV system is split into Transfer plasmid, Rep (Rep52/40+Rep78/68), Helper (E2A+E4orf6 and VARNA), and Cap gene containing plasmids. The expression of each gene required for AAV production is induced through the addition of doxycycline. Sleeping Beauty transposon IR/DRs and antibiotic selection cassettes are included to enhance the integration efficiency and enable efficient selection of cells with genomic integration events, respectively.

FIG. 3 shows results from production of AAV using pTRE3G induced expression of Rep+Cap, and E2A+E4orf6 and VA RNA. Dox-induction leads to ˜27 fold increase in AAV titer.

FIG. 4 shows a plasmid schematic for refactored AAV stable integration plasmids. Rep, Cap, and Helper genes are all under the control of doxycycline inducible promoters. Rep68/78 was modified to remove the Rep52 start codon and in Rep78 the Rep68/40 splice site was removed. Rep52 was modified to remove the Rep40 splice site. All sequences were refactored to minimize promoter elements. Rep52 and Rep78 genes are transcriptionally (IRES) or translationally (P2A) linked. Coding DNA sequences for helper genes E2A and E4orf6 are transcriptionally (IRES) or translationally (P2A) linked. Cap gene VP1 is modified to remove start codons for VP2/3/AAP and expressed on the same plasmid as wild type VP2/3/AAP gene fragment.

FIG. 5 shows a plasmid schematic for HSV helper stable integration plasmids. All elements of the HSV-1 replication complex are under doxycycline inducible control. Genes encoding the HSV-1 polymerase (UL30 and UL42) are transcriptionally linked via an IRES sequence. HSV-1 endonuclease (UL12) is constitutively expressed from the hEF1a promoter. ICP0, UL12, ICP4, and ICP22 are also encoded under control of pTRE3G promoters. UL12 and ICP4 are also transcriptionally linked via an IRES.

FIG. 6 shows results from production of AAV using pTRE3G induced expression of Rep+Cap, HSV-1 polymerase, helicase-primase elements, and with and without UL12 endonuclease. Dox-induction leads to ˜18 fold increase in AAV titer.

### DETAILED DESCRIPTION

The production of viral vectors normally entails transient transfection of plasmids into cell culture. However, stable integration of genes necessary to produce therapeutic viral vectors into the genome offers several advantages compared to traditional production via transient transfection. Since cells amplify the viral genes during their own cell division, large quantities of DNA and transfection reagent no longer need to be procured for the transfection process, reducing costs. Also, since the DNA is already within the nucleus, viral titers may be higher and more consistent due to minimal numbers of “untransfected” cells and reduced variation associated with transfection steps. The simpler production process also saves time.

However, several genes required for adeno-associated viral (AAV) vector production have been demonstrated by others to be cytostatic or cytotoxic, namely Rep, E2A and E4. The cytotoxic and cytostatic nature of these proteins has hampered the development of stable AAV producer cell lines in the widely used HEK293 cell line, since the native expression of adenovirus E1 genes in HEK293 cells upregulates expression of these toxic genes. Cells stably transfected with these genes fail to survive selection steps or have silenced expression, resulting in an inability to produce relevant quantities of AAV.

Described herein are AAV vector production systems and HSV-helper systems. Also described herein are kits comprising an AAV vector production system and/or an HSV-helper system. Finally, engineered cells comprising an AAV vector production system (or at least a portion thereof) and/or an HSV-helper system are described, as well as methods of using the engineered cells for AAV vector production.

### I. AAV Vector Production Systems

In some aspects, the disclosure relates to AAV vector production systems. An AAV vector production system, as described herein, comprises one or more polynucleic acids that collectively encode the gene products required for generation of an AAV vector in a recombinant host cell (or an “engineered cell” as described herein). The AAV vector production systems described herein comprise one or more polynucleotides that collectively encode for AAV gene products: Rep52 (or a functional variant thereof) or Rep40 (or a functional variant thereof); Rep78 (or a functional variant thereof) or Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); and AAP (or a functional variant thereof). In some embodiments, an AAV vector production system comprises one or more polynucleotides that collectively encode for: Rep52 (or a functional variant thereof); Rep40 (or a functional variant thereof); Rep78 (or a functional variant thereof); Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); and AAP (or a functional variant thereof). In some embodiments, an AAV vector production system further comprises a polynucleotide encoding for MAAP (or a functional variant thereof)—which is not essential for generation of an AAV vector in a recombinant host cell.

In some embodiments, an AAV vector production system is (i.e., the gene products of the viral vector component are) encoded on a single polynucleic acid. In other embodiments, multiple polynucleic acids collectively comprise an AAV vector production system (i.e., at least two of the gene products of the viral vector component are encoded on different polynucleic acids). For example, an AAV vector production system may comprise at least 2, at least 3, at least 4, or at least 5 polynucleic acids. In some embodiments, an AAV vector production system comprises 2, 3, 4, or 5 polynucleic acids. Exemplary AAV production system architectures are provided below (Part IC).

Rep52 is a protein involved in AAV genome packaging. In some embodiments, an AAV vector production system comprises a polynucleic acid encoding for Rep52. In some embodiments, Rep52 comprises the amino acid sequence of SEQ ID NO: 18. In some embodiments, an AAV vector production system comprises a polynucleic acid encoding for a functional variant of Rep52. A functional variant of Rep52 comprises at least 80% identity (at least 85%, at least 90%, at least 95%, at least 96%, at least 97%, at least 98%, or at least 99% identity) with SEQ ID NO: 18 and maintains at least 80% of the function (at least 85%, at least 90%, at least 95%, at least 96%, at least 97%, at least 98%, or at least 99% of the function) of Rep52 (that is, its function in AAV genome packaging). In some embodiments, the nucleic acid sequence encoding for Rep52 lacks the Rep40 splice site. See Stutika et al. J Virol. 2015 Nov. 11; 90(3):1278-89 the entire contents of which are incorporated herein by reference. In some embodiments, the nucleic acid sequence encoding for Rep52 and lacking the Rep40 splice site comprises the nucleic acid sequence of SEQ ID NO: 19 (“SS-Rep52”) or a similar nucleic acid sequence that lacks the Rep40 splice site and encodes Rep52 (or a Rep52 variant, as described above).

Methods of determining the extent of identity between two sequences (e.g., two amino acid sequences or two polynucleic acids) are known to those having ordinary skill in the art. One exemplary method is the use of Basic Local Alignment Search Tool (BLAST®) software with default parameters (blast.ncbi.nlm.nih.gov/Blast.cgi).

Rep40 is a protein involved in AAV genome packaging. In some embodiments, an AAV vector production system comprises a polynucleic acid encoding for Rep40. In some embodiments, Rep40 comprises the amino acid sequence of SEQ ID NO: 20. In some embodiments, an AAV vector production system comprises a polynucleic acid encoding for a functional variant of Rep40. A functional variant of Rep40 comprises at least 80% identity (at least 85%, at least 90%, at least 95%, at least 96%, at least 97%, at least 98%, or at least 99% identity) with SEQ ID NO: 20 and maintains at least 80% of the function (at least 85%, at least 90%, at least 95%, at least 96%, at least 97%, at least 98%, or at least 99% of the function) of Rep40 (that is, its function in AAV genome packaging).

Rep78 is a protein involved in AAV genome replication. In some embodiments, an AAV vector production system comprises a polynucleic acid encoding for Rep78. In some embodiments, Rep78 comprises the amino acid sequence of SEQ ID NO: 21. In some embodiments, an AAV vector production system comprises a polynucleic acid encoding for a functional variant of Rep78. A functional variant of Rep78 comprises at least 80% identity (at least 85%, at least 90%, at least 95%, at least 96%, at least 97%, at least 98%, or at least 99% identity) with SEQ ID NO: 21 and maintains at least 80% of the function (at least 85%, at least 90%, at least 95%, at least 96%, at least 97%, at least 98%, or at least 99% of the function) of Rep78 (that is, its function in AAV genome replication). An exemplary functional variant of Rep78 is one that lacks a Rep52 start codon. Such a functional variant may comprise the amino acid sequence of SEQ ID NO: 22 (“SC/SS-Rep78”). In some embodiments, the nucleic acid sequence encoding for Rep78 lacks the Rep68/40 splice site. See Stutika et al. J Virol. 2015 Nov. 11; 90(3):1278-89 the entire contents of which are incorporated herein by reference. In some embodiments, the nucleic acid sequence encoding for Rep78 lacks the Rep52 start codon and the Rep68/40 splice site. In some embodiments, the nucleic acid sequence encoding for Rep78 and lacking the Rep52 start codon and the Rep68/40 splice site comprises the nucleic acid sequence of SEQ ID NO: 23 or a similar nucleic acid sequence that lacks the Rep52 start codon and the Rep68/40 splice site and encodes a Rep78 variant (as described above).

Rep68 is a protein involved in AAV genome replication. In some embodiments, an AAV vector production system comprises a polynucleic acid encoding for Rep68. In some embodiments, Rep68 comprises the amino acid sequence of SEQ ID NO: 26. In some embodiments, an AAV vector production system comprises a polynucleic acid encoding for a functional variant of Rep68. A functional variant of Rep78 comprises at least 80% identity (at least 85%, at least 90%, at least 95%, at least 96%, at least 97%, at least 98%, or at least 99% identity) with SEQ ID NO: 26 and maintains at least 80% of the function (at least 85%, at least 90%, at least 95%, at least 96%, at least 97%, at least 98%, or at least 99% of the function) of Rep68 (that is, its function in AAV genome replication). An exemplary functional variant of Rep68 is one that lacks the Rep52 start codon. Such a functional variant may comprise the amino acid sequence of SEQ ID NO: 27 (“SC-Rep68”). In some embodiments, the nucleic acid sequence encoding for Rep78 lacks the Rep52 start codon. In some embodiments, the nucleic acid sequence encoding for Rep78 and lacking the Rep52 start codon comprises the nucleic acid sequence of SEQ ID NO: 28 or a similar nucleic acid sequence that lacks the Rep52 start codon and encodes a Rep68 variant (as described above).

E2A is a protein involved in AAV genome replication. In some embodiments, an AAV vector production system comprises a polynucleic acid encoding for E2A. In some embodiments, E2A comprises the amino acid sequence of SEQ ID NO: 29. In some embodiments, an AAV vector production system comprises a polynucleic acid encoding for a functional variant of E2A. A functional variant of E2A comprises at least 80% identity (at least 85%, at least 90%, at least 95%, at least 96%, at least 97%, at least 98%, or at least 99% identity) with SEQ ID NO: 29 and maintains at least 80% of the function (at least 85%, at least 90%, at least 95%, at least 96%, at least 97%, at least 98%, or at least 99% of the function) of E2A (that is, its function in AAV genome replication).

E4Orf6 is a protein involved in AAV genome replication. In some embodiments, an AAV vector production system comprises a polynucleic acid encoding for E4Orf6. In some embodiments, E4Orf6 comprises the amino acid sequence of SEQ ID NO: 35. In some embodiments, an AAV vector production system comprises a polynucleic acid encoding for a functional variant of E4Orf6. A functional variant of E4Orf6 comprises at least 80% identity (at least 85%, at least 90%, at least 95%, at least 96%, at least 97%, at least 98%, or at least 99% identity) with SEQ ID NO: 35 and maintains at least 80% of the function (at least 85%, at least 90%, at least 95%, at least 96%, at least 97%, at least 98%, or at least 99% of the function) of E4Orf6 (that is, its function in AAV genome replication). In some embodiments, the nucleic acid sequence encoding for E4Orf6 lacks a splice site, such as the E4orf6sf “splice-fix” variant reported in Querido E., Identification and elimination of an aberrant splice product from cDNAs encoding the human adenovirus type 5 E4orf6 protein, Virology. 2000 Sep. 30; 275(2):263-6. In some embodiments, the nucleic acid sequence encoding for E4Orf6 that lacks the splice site comprises the nucleic acid sequence of SEQ ID NO: 36 (“SS-E40RF6”) or a similar nucleic acid sequence that lacks the splice site and that encodes E4Orf6 (or a variant thereof, as described above).

VARNA is a non-coding RNA that stimulates expression of AAV proteins. In some embodiments, an AAV vector production system comprises a polynucleic acid encoding for VARNA. In some embodiments, VARNA comprises the nucleic acid sequence of SEQ ID NO: 38. In some embodiments, an AAV vector production system comprises a polynucleic acid encoding for a functional variant of VARNA. A functional variant of VARNA comprises at least 80% identity (at least 85%, at least 90%, at least 95%, at least 96%, at least 97%, at least 98%, or at least 99% identity) with SEQ ID NO: 38 and maintains at least 80% of the function (at least 85%, at least 90%, at least 95%, at least 96%, at least 97%, at least 98%, or at least 99% of the function) of VARNA (that is, its function in stimulating expression of AAV proteins).

VP1 is an AAV capsid protein. In some embodiments, an AAV vector production system comprises a polynucleic acid encoding for VP1. In some embodiments, VP1 comprises the amino acid sequence of SEQ ID NO: 30. In some embodiments, an AAV vector production system comprises a polynucleic acid encoding for a functional variant of VP1. A functional variant of VP1 comprises at least 80% identity (at least 85%, at least 90%, at least 95%, at least 96%, at least 97%, at least 98%, or at least 99% identity) with SEQ ID NO: 30 and maintains at least 80% of the function (at least 85%, at least 90%, at least 95%, at least 96%, at least 97%, at least 98%, or at least 99% of the function) of VP1 (that is, its function as an AAV capsid protein). An exemplary functional variant of VP1 is one that lacks a start codon for VP2, VP3, AAP, or a combination thereof. In some embodiments, a variant of VP1 lacks a start codon for VP2, VP3, and AAP and comprises the amino acid sequence of SEQ ID NO: 31 (“SC-VP1”). In some embodiments, the nucleic acid sequence encoding for VP1 lacks a start codon for VP2, VP3, AAP, or a combination thereof. In some embodiments, the nucleic acid sequence encoding for VP1 that lacks start codons for VP2, VP3, and AAP comprises the nucleic acid sequence of SEQ ID NO: 32 or a similar nucleic acid sequence that lacks the start codons for VP2, VP3, and AAP and that encodes a VP1 variant (as described above).

VP2 is an AAV capsid protein. In some embodiments, an AAV vector production system comprises a polynucleic acid encoding for VP2. In some embodiments, VP2 comprises the amino acid sequence of SEQ ID NO: 33. In some embodiments, an AAV vector production system comprises a polynucleic acid encoding for a functional variant of VP2. A functional variant of VP2 comprises at least 80% identity (at least 85%, at least 90%, at least 95%, at least 96%, at least 97%, at least 98%, or at least 99% identity) with SEQ ID NO: 33 and maintains at least 80% of the function (at least 85%, at least 90%, at least 95%, at least 96%, at least 97%, at least 98%, or at least 99% of the function) of VP2 (that is, its function as an AAV capsid protein).

VP3 is an AAV capsid protein. In some embodiments, an AAV vector production system comprises a polynucleic acid encoding for VP3. In some embodiments, VP3 comprises the amino acid sequence of SEQ ID NO: 34. In some embodiments, an AAV vector production system comprises a polynucleic acid encoding for a functional variant of VP3. A functional variant of VP3 comprises at least 80% identity (at least 85%, at least 90%, at least 95%, at least 96%, at least 97%, at least 98%, or at least 99% identity) with SEQ ID NO: 34 and maintains at least 80% of the function (at least 85%, at least 90%, at least 95%, at least 96%, at least 97%, at least 98%, or at least 99% of the function) of VP3 (that is, its function as an AAV capsid protein).

AAP is protein that promotes AAV capsid assembly. In some embodiments, an AAV vector production system comprises a polynucleic acid encoding for AAP. In some embodiments, AAP comprises the amino acid sequence of SEQ ID NO: 37. In some embodiments, an AAV vector production system comprises a polynucleic acid encoding for a functional variant of AAP. A functional variant of APP comprises at least 80% identity (at least 85%, at least 90%, at least 95%, at least 96%, at least 97%, at least 98%, or at least 99% identity) with SEQ ID NO: 37 and maintains at least 80% of the function (at least 85%, at least 90%, at least 95%, at least 96%, at least 97%, at least 98%, or at least 99% of the function) of AAP (that is, its function in promoting AAV capsid assembly).

MAAP is a membrane-associated accessory protein that is beneficial for AAV replication/infection. In some embodiments, an AAV vector production system comprises a polynucleic acid encoding for MAAP. In some embodiments, MAAP comprises the amino acid sequence of SEQ ID NO: 49. In some embodiments, an AAV vector production system comprises a polynucleic acid encoding for a functional variant of MAAP. A functional variant of MAPP comprises at least 80% identity (at least 85%, at least 90%, at least 95%, at least 96%, at least 97%, at least 98%, or at least 99% identity) with SEQ ID NO: 49 and maintains at least 80% of the function (at least 85%, at least 90%, at least 95%, at least 96%, at least 97%, at least 98%, or at least 99% of the function) of MAAP (that is, its function in promoting replication/infection).

The AAV production systems described herein comprise at least one expression cassette. As used herein, the term “expression cassette” refers to a polynucleic acid sequence encoding a nucleic acid sequence of a promoter that is operably linked to a nucleic acid encoding a product (e.g., an RNA product(s), VARNA (or a functional variant thereof), and/or a polypeptide product(s), such as Rep52 (or a functional variant thereof), Rep40 (or a functional variant thereof), Rep78 (or a functional variant thereof), Rep68 (or a functional variant thereof), E2A (or a functional variant thereof), E4Orf6 (or a functional variant thereof), VP1 (or a functional variant thereof), VP2 (or a functional variant thereof), VP3 (or a functional variant thereof), AAP (or a functional variant thereof), MAAP (or a functional variant thereof), or any combination thereof). In some embodiments, multiple products are encoded within a single expression cassette. For example, in some embodiments, a single promoter drives expression of a polycistronic RNA encoding for multiple products (an RNA product(s) and/or a polypeptide product(s)). A polycistronic RNA may comprise a nucleic acid sequence of an internal ribosomal entry site (IRES) and/or a nucleic acid sequence of a viral 2A peptide (V2A).

An IRES may comprises the nucleic acid sequence of SEQ ID NO: 45:

CCCCTCTCCCTCCCCCCCCCCTAACGTTACTGGCCGAAGCCGCTTGGAA
TAAGGCCGGTGTGCGTTTGTCTATATGTTATTTTCCACCATATTGCCGT
CTTTTGGCAATGTGAGGGCCCGGAAACCTGGCCCTGTCTTCTTGACGAG
CATTCCTAGGGGTCTTTCCCCTCTCGCCAAAGGAATGCAAGGTCTGTTG
AATGTCGTGAAGGAAGCAGTTCCTCTGGAAGCTTCTTGAAGACAAACAA
CGTCTGTAGCGACCCTTTGCAGGCAGCGGAACCCCCCACCTGGCGACAG
GTGCCTCTGCGGCCAAAAGCCACGTGTATAAGATACACCTGCAAAGGCG
GCACAACCCCAGTGCCACGTTGTGAGTTGGATAGTTGTGGAAAGAGTCA
AATGGCTCTCCTCAAGCGTATTCAACAAGGGGCTGAAGGATGCCCAGAA
GGTACCCCATTGTATGGGATCTGATCTGGGGCCTCGGTGCACATGCTTT
ACATGTGTTTAGTCGAGGTTAAAAAAACGTCTAGGCCCCCCGAACCACG
GGGACGTGGTTTTCCTTTGAAAAACACGATGATAATATG

An IRES may comprise the nucleic acid sequence of SEQ ID NO: 46:

CCCCCCCCCCTAACGTTACTGGCCGAAGCCGCTTGGAATAAGGCCGGTG
TGCGTTTGTCTATATGTTATTTTCCACCATATTGCCGTCTTTTGGCAAT
GTGAGGGCCCGGAAACCTGGCCCTGTCTTCTTGACGAGCATTCCTAGGG
GTCTTTCCCCTCTCGCCAAAGGAATGCAAGGTCTGTTGAATGTCGTGAA
GGAAGCAGTTCCTCTGGAAGCTTCTTGAAGACAAACAACGTCTGTAGCG
ACCCTTTGCAGGCAGCGGAACCCCCCACCTGGCGACAGGTGCCTCTGCG
GCCAAAAGCCACGTGTATAAGATACACCTGCAAAGGCGGCACAACCCCA
GTGCCACGTTGTGAGTTGGATAGTTGTGGAAAGAGTCAAATGGCTCTCC
TCAAGCGTATTCAACAAGGGGCTGAAGGATGCCCAGAAGGTACCCCATT
GTATGGGATCTGATCTGGGGCCTCGGTGCACATGCTTTACATGTGTTTA
GTCGAGGTTAAAAAAACGTCTAGGCCCCCCGAACCACGGGGACGTGGTT
TTCCTTTGAAAAACACGATGATAATAGTTATC

A viral 2A peptide may comprise the amino acid sequence of

(SEQ ID NO: 47)
ATNFSLLKQAGDVEENPGP
or
(SEQ ID NO: 48)
EGRGSLLTCGDVEENPGP.

In some embodiments, a polynucleic acid of an AAV vector production system comprises multiple cassettes. In instances when an AAV vector production system comprises multiple cassettes, the cassettes may be positioned in various orientations. For example, in some embodiments, each of the cassettes are encoded in the same orientation (i.e., encoding on the same strand). In other embodiments, at least one cassette is encoded in an opposite orientation (i.e., encoding on the opposite strand). Cassettes positioned in an opposite orientation may have convergent expression (→←) or divergent expression (←→). In some embodiments, the expression cassettes are positioned in an alternating orientation.

As described herein, a promoter is “operably linked” to a nucleic acid coding sequence when the position of the promoter relative to the nucleic acid coding sequence is such that binding of a transcriptional activator to the promoter can induce expression of the coding sequence. A promoter of an expression cassette may be a constitutive promoter or an inducible promoter.

A promoter may be a constitutive promoter (i.e., an unregulated promoter that allows for continual transcription). Examples of constitutive promoters are known in the art and include, but are not limited to, cytomegalovirus (CMV) promoters, elongation factor 1α (EF1α) promoters, simian vacuolating virus 40 (SV40) promoters, ubiquitin-C (UBC) promoters, U6 promoters, and phosphoglycerate kinase (PGK) promoters. See e.g., Ferreira et al., Tuning gene expression with synthetic upstream open reading frames. Proc. Natl. Acad. Sci. U.S.A. 2013 July; 110(28): 11284-89; Pub. No.: US 2014/377861 A1—the entireties of which are incorporated herein by reference.

Alternatively, a promoter may be an inducible promoter (i.e., only activates transcription under specific circumstances). An inducible promoter may be a chemically inducible promoter, a temperature inducible promoter, or a light inducible promoter. Examples of chemically inducible promoters are known in the art and include, but are not limited to, tetracycline/doxycycline inducible promoters, cumate inducible promoters, ABA inducible promoters, CRY2-CIB1 inducible promoters, DAPG inducible promoters, and mifepristone inducible promoters. See e.g., Stanton et al., ACS Synth. Biol. 2014 Dec. 19; 3(12): 880-91; Liang et al., Sci. Signal. 2011 Mar. 15; 4(164): rs2; U.S. Pat. No. 7,745,592 B2; U.S. Pat. No. 7,935,788 B2—the entireties of which are incorporated herein by reference. A chemically inducible promoter may comprise the nucleic acid sequence of one or more of SEQ ID NOs: 1-9.

### A. Selectable Markers

As described above, an AAV vector production system, as described herein, comprises one or more polynucleic acids that collectively encode the gene products required for generation of an AAV vector in a recombinant host cell (or an “engineered cell” as described herein). In some embodiments, one or more of the polynucleic acids of an AAV vector production system comprises an expression cassette comprising: (i) a nucleic acid sequence of a promoter (constitutive or inducible, as described herein); and (ii) a nucleic acid sequence encoding a selectable maker. In some embodiments, each of the polynucleic acids of a AAV vector production system comprises a selectable maker. In some embodiments, each polynucleic acid of an AAV vector production system comprises a nucleic acid sequence of a distinct selectable marker.

As used herein, the term “selectable marker” refers to a protein that—when introduced into or expressed in a cell—confers a trait that is suitable for selection.

A selectable marker may be a fluorescent protein. Examples of fluorescent proteins are known in the art (e.g., TagBFP, EBFP2, EGFP, EYFP, mKO2, or Sirius). See e.g., U.S. Pat. No. 5,874,304; Patent No.: EP 0969284 A1; Pub. No.: US 2010/167394 A—the entireties of which are incorporated here by reference.

Alternatively, or in addition, a selectable marker may be an antibiotic resistance protein. Examples of antibiotic resistance proteins are known in the art (e.g., facilitating puromycin, hygromycin, neomycin, zeocin, blasticidin, or phleomycin selection). See e.g., Pub. No.: WO 1997/15668 A2; Pub. No.: WO 1997/43900 A1—the entireties of which are incorporated here by reference.

### B. Inducible AAV Vector Production Systems

In some embodiments, an AAV vector production system described herein comprises one or more polynucleotides that collectively encode for: Rep52 (or a functional variant thereof) or Rep40 (or a functional variant thereof); Rep78 (or a functional variant thereof) or Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); and AAP (or a functional variant thereof); optionally MAAP (or a functional variant thereof); at least one of which is operably linked to a chemically inducible promoter. In some embodiments, an AAV vector production system comprises one or more polynucleotides that collectively encode for: Rep52 (or a functional variant thereof); Rep40 (or a functional variant thereof); Rep78 (or a functional variant thereof); Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); and AAP (or a functional variant thereof); optionally MAAP (or a functional variant thereof); each of which is operably linked to a chemically inducible promoter.

In any of the embodiments described in this section, a chemically inducible promoter may comprise a tetracycline/doxycycline inducible promoter, a cumate inducible promoter, an ABA inducible promoter, a CRY2-CIB1 inducible promoter, a DAPG inducible promoter, a mifepristone inducible promoter, or a combination thereof. In some embodiments, a chemically inducible promoter comprises the nucleic acid sequence of one or more of SEQ ID NOs: 1-9. In some embodiments, a chemically inducible promoter comprises the nucleic acid sequence of SEQ ID NO: 1. In some embodiments, a chemically inducible promoter comprises the nucleic acid sequence of SEQ ID NO: 2. In some embodiments, a chemically inducible promoter comprises the nucleic acid sequence of SEQ ID NO: 3. In some embodiments, a chemically inducible promoter comprises the nucleic acid sequence of SEQ ID NO: 4. In some embodiments, a chemically inducible promoter comprises the nucleic acid sequence of SEQ ID NO: 5. In some embodiments, a chemically inducible promoter comprises the nucleic acid sequence of SEQ ID NO: 6. In some embodiments, a chemically inducible promoter comprises the nucleic acid sequence of SEQ ID NO: 7. In some embodiments, a chemically inducible promoter comprises the nucleic acid sequence of SEQ ID NO: 8. In some embodiments, a chemically inducible promoter comprises the nucleic acid sequence of SEQ ID NO: 9.

In some embodiments, the nucleic acid sequence encoding Rep52 (or a functional variant thereof) is operably linked to a chemically inducible promoter (as described herein); optionally wherein: the nucleic acid sequence encoding Rep40 (or a functional variant thereof) is operably linked to a chemically inducible promoter (as described herein); and/or the nucleic acid sequence encoding Rep78 (or a functional variant thereof) is operably linked to a chemically inducible promoter (as described herein); and/or the nucleic acid sequence encoding Rep68 (or a functional variant thereof) is operably linked to a chemically inducible promoter (as described herein); and/or the nucleic acid sequence encoding E2A (or a functional variant thereof) is operably linked to a chemically inducible promoter (as described herein); and/or the nucleic acid sequence encoding E4Orf6 (or a functional variant thereof) is operably linked to a chemically inducible promoter (as described herein); and/or the nucleic acid sequence encoding VARNA (or a functional variant thereof) is operably linked to a chemically inducible promoter (as described herein); and/or the nucleic acid sequence encoding VP1 (or a functional variant thereof) is operably linked to a chemically inducible promoter (as described herein); and/or the nucleic acid sequence encoding VP2 (or a functional variant thereof) is operably linked to a chemically inducible promoter (as described herein); and/or the nucleic acid sequence encoding VP3 (or a functional variant thereof) is operably linked to a chemically inducible promoter (as described herein); and/or the nucleic acid sequence encoding AAP (or a functional variant thereof) is operably linked to a chemically inducible promoter (as described herein); and/or the nucleic acid sequence encoding MAAP (or a functional variant thereof) is operably linked to a chemically inducible promoter (as described herein).

In some embodiments, the nucleic acid sequence encoding Rep40 (or a functional variant thereof) is operably linked to a chemically inducible promoter (as described herein); optionally wherein: the nucleic acid sequence encoding Rep52 (or a functional variant thereof) is operably linked to a chemically inducible promoter (as described herein); and/or the nucleic acid sequence encoding Rep78 (or a functional variant thereof) is operably linked to a chemically inducible promoter (as described herein); and/or the nucleic acid sequence encoding Rep68 (or a functional variant thereof) is operably linked to a chemically inducible promoter (as described herein); and/or the nucleic acid sequence encoding E2A (or a functional variant thereof) is operably linked to a chemically inducible promoter (as described herein); and/or the nucleic acid sequence encoding E4Orf6 (or a functional variant thereof) is operably linked to a chemically inducible promoter (as described herein); and/or the nucleic acid sequence encoding VARNA (or a functional variant thereof) is operably linked to a chemically inducible promoter (as described herein); and/or the nucleic acid sequence encoding VP1 (or a functional variant thereof) is operably linked to a chemically inducible promoter (as described herein); and/or the nucleic acid sequence encoding VP2 (or a functional variant thereof) is operably linked to a chemically inducible promoter (as described herein); and/or the nucleic acid sequence encoding VP3 (or a functional variant thereof) is operably linked to a chemically inducible promoter (as described herein); and/or the nucleic acid sequence encoding AAP (or a functional variant thereof) is operably linked to a chemically inducible promoter (as described herein); and/or the nucleic acid sequence encoding MAAP (or a functional variant thereof) is operably linked to a chemically inducible promoter (as described herein).

In some embodiments, the nucleic acid sequence encoding Rep78 (or a functional variant thereof) is operably linked to a chemically inducible promoter (as described herein); optionally wherein: the nucleic acid sequence encoding Rep40 (or a functional variant thereof) is operably linked to a chemically inducible promoter (as described herein); and/or the nucleic acid sequence encoding Rep52 (or a functional variant thereof) is operably linked to a chemically inducible promoter (as described herein); and/or the nucleic acid sequence encoding Rep68 (or a functional variant thereof) is operably linked to a chemically inducible promoter (as described herein); and/or the nucleic acid sequence encoding E2A (or a functional variant thereof) is operably linked to a chemically inducible promoter (as described herein); and/or the nucleic acid sequence encoding E4Orf6 (or a functional variant thereof) is operably linked to a chemically inducible promoter (as described herein); and/or the nucleic acid sequence encoding VARNA (or a functional variant thereof) is operably linked to a chemically inducible promoter (as described herein); and/or the nucleic acid sequence encoding VP1 (or a functional variant thereof) is operably linked to a chemically inducible promoter (as described herein); and/or the nucleic acid sequence encoding VP2 (or a functional variant thereof) is operably linked to a chemically inducible promoter (as described herein); and/or the nucleic acid sequence encoding VP3 (or a functional variant thereof) is operably linked to a chemically inducible promoter (as described herein); and/or the nucleic acid sequence encoding AAP (or a functional variant thereof) is operably linked to a chemically inducible promoter (as described herein); and/or the nucleic acid sequence encoding MAAP (or a functional variant thereof) is operably linked to a chemically inducible promoter (as described herein).

In some embodiments, the nucleic acid sequence encoding Rep78 (or a functional variant thereof) is operably linked to a chemically inducible promoter (as described herein); optionally wherein: the nucleic acid sequence encoding Rep40 (or a functional variant thereof) is operably linked to a chemically inducible promoter (as described herein); and/or the nucleic acid sequence encoding Rep52 (or a functional variant thereof) is operably linked to a chemically inducible promoter (as described herein); and/or the nucleic acid sequence encoding Rep68 (or a functional variant thereof) is operably linked to a chemically inducible promoter (as described herein); and/or the nucleic acid sequence encoding E2A (or a functional variant thereof) is operably linked to a chemically inducible promoter (as described herein); and/or the nucleic acid sequence encoding E4Orf6 (or a functional variant thereof) is operably linked to a chemically inducible promoter (as described herein); and/or the nucleic acid sequence encoding VARNA (or a functional variant thereof) is operably linked to a chemically inducible promoter (as described herein); and/or the nucleic acid sequence encoding VP1 (or a functional variant thereof) is operably linked to a chemically inducible promoter (as described herein); and/or the nucleic acid sequence encoding VP2 (or a functional variant thereof) is operably linked to a chemically inducible promoter (as described herein); and/or the nucleic acid sequence encoding VP3 (or a functional variant thereof) is operably linked to a chemically inducible promoter (as described herein); and/or the nucleic acid sequence encoding AAP (or a functional variant thereof) is operably linked to a chemically inducible promoter (as described herein); and/or the nucleic acid sequence encoding MAAP (or a functional variant thereof) is operably linked to a chemically inducible promoter (as described herein).

In some embodiments, the nucleic acid sequence encoding Rep68 (or a functional variant thereof) is operably linked to a chemically inducible promoter (as described herein); optionally wherein: the nucleic acid sequence encoding Rep40 (or a functional variant thereof) is operably linked to a chemically inducible promoter (as described herein); and/or the nucleic acid sequence encoding Rep78 (or a functional variant thereof) is operably linked to a chemically inducible promoter (as described herein); and/or the nucleic acid sequence encoding Rep52 (or a functional variant thereof) is operably linked to a chemically inducible promoter (as described herein); and/or the nucleic acid sequence encoding E2A (or a functional variant thereof) is operably linked to a chemically inducible promoter (as described herein); and/or the nucleic acid sequence encoding E4Orf6 (or a functional variant thereof) is operably linked to a chemically inducible promoter (as described herein); and/or the nucleic acid sequence encoding VARNA (or a functional variant thereof) is operably linked to a chemically inducible promoter (as described herein); and/or the nucleic acid sequence encoding VP1 (or a functional variant thereof) is operably linked to a chemically inducible promoter (as described herein); and/or the nucleic acid sequence encoding VP2 (or a functional variant thereof) is operably linked to a chemically inducible promoter (as described herein); and/or the nucleic acid sequence encoding VP3 (or a functional variant thereof) is operably linked to a chemically inducible promoter (as described herein); and/or the nucleic acid sequence encoding AAP (or a functional variant thereof) is operably linked to a chemically inducible promoter (as described herein); and/or the nucleic acid sequence encoding MAAP (or a functional variant thereof) is operably linked to a chemically inducible promoter (as described herein).

In some embodiments, the nucleic acid sequence encoding E2A (or a functional variant thereof) is operably linked to a chemically inducible promoter (as described herein); optionally wherein: the nucleic acid sequence encoding Rep40 (or a functional variant thereof) is operably linked to a chemically inducible promoter (as described herein); and/or the nucleic acid sequence encoding Rep78 (or a functional variant thereof) is operably linked to a chemically inducible promoter (as described herein); and/or the nucleic acid sequence encoding Rep68 (or a functional variant thereof) is operably linked to a chemically inducible promoter (as described herein); and/or the nucleic acid sequence encoding Rep52 (or a functional variant thereof) is operably linked to a chemically inducible promoter (as described herein); and/or the nucleic acid sequence encoding E4Orf6 (or a functional variant thereof) is operably linked to a chemically inducible promoter (as described herein); and/or the nucleic acid sequence encoding VARNA (or a functional variant thereof) is operably linked to a chemically inducible promoter (as described herein); and/or the nucleic acid sequence encoding VP1 (or a functional variant thereof) is operably linked to a chemically inducible promoter (as described herein); and/or the nucleic acid sequence encoding VP2 (or a functional variant thereof) is operably linked to a chemically inducible promoter (as described herein); and/or the nucleic acid sequence encoding VP3 (or a functional variant thereof) is operably linked to a chemically inducible promoter (as described herein); and/or the nucleic acid sequence encoding AAP (or a functional variant thereof) is operably linked to a chemically inducible promoter (as described herein); and/or the nucleic acid sequence encoding MAAP (or a functional variant thereof) is operably linked to a chemically inducible promoter (as described herein).

In some embodiments, the nucleic acid sequence encoding E4Orf6 (or a functional variant thereof) is operably linked to a chemically inducible promoter (as described herein); optionally wherein: the nucleic acid sequence encoding Rep40 (or a functional variant thereof) is operably linked to a chemically inducible promoter (as described herein); and/or the nucleic acid sequence encoding Rep78 (or a functional variant thereof) is operably linked to a chemically inducible promoter (as described herein); and/or the nucleic acid sequence encoding Rep68 (or a functional variant thereof) is operably linked to a chemically inducible promoter (as described herein); and/or the nucleic acid sequence encoding E2A (or a functional variant thereof) is operably linked to a chemically inducible promoter (as described herein); and/or the nucleic acid sequence encoding Rep52 (or a functional variant thereof) is operably linked to a chemically inducible promoter (as described herein); and/or the nucleic acid sequence encoding VARNA (or a functional variant thereof) is operably linked to a chemically inducible promoter (as described herein); and/or the nucleic acid sequence encoding VP1 (or a functional variant thereof) is operably linked to a chemically inducible promoter (as described herein); and/or the nucleic acid sequence encoding VP2 (or a functional variant thereof) is operably linked to a chemically inducible promoter (as described herein); and/or the nucleic acid sequence encoding VP3 (or a functional variant thereof) is operably linked to a chemically inducible promoter (as described herein); and/or the nucleic acid sequence encoding AAP (or a functional variant thereof) is operably linked to a chemically inducible promoter (as described herein); and/or the nucleic acid sequence encoding MAAP (or a functional variant thereof) is operably linked to a chemically inducible promoter (as described herein).

In some embodiments, the nucleic acid sequence encoding VARNA (or a functional variant thereof) is operably linked to a chemically inducible promoter (as described herein); optionally wherein: the nucleic acid sequence encoding Rep40 (or a functional variant thereof) is operably linked to a chemically inducible promoter (as described herein); and/or the nucleic acid sequence encoding Rep78 (or a functional variant thereof) is operably linked to a chemically inducible promoter (as described herein); and/or the nucleic acid sequence encoding Rep68 (or a functional variant thereof) is operably linked to a chemically inducible promoter (as described herein); and/or the nucleic acid sequence encoding E2A (or a functional variant thereof) is operably linked to a chemically inducible promoter (as described herein); and/or the nucleic acid sequence encoding E4Orf6 (or a functional variant thereof) is operably linked to a chemically inducible promoter (as described herein); and/or the nucleic acid sequence encoding Rep52 (or a functional variant thereof) is operably linked to a chemically inducible promoter (as described herein); and/or the nucleic acid sequence encoding VP1 (or a functional variant thereof) is operably linked to a chemically inducible promoter (as described herein); and/or the nucleic acid sequence encoding VP2 (or a functional variant thereof) is operably linked to a chemically inducible promoter (as described herein); and/or the nucleic acid sequence encoding VP3 (or a functional variant thereof) is operably linked to a chemically inducible promoter (as described herein); and/or the nucleic acid sequence encoding AAP (or a functional variant thereof) is operably linked to a chemically inducible promoter (as described herein); and/or the nucleic acid sequence encoding MAAP (or a functional variant thereof) is operably linked to a chemically inducible promoter (as described herein).

In some embodiments, the nucleic acid sequence encoding VP1 (or a functional variant thereof) is operably linked to a chemically inducible promoter (as described herein); optionally wherein: the nucleic acid sequence encoding Rep40 (or a functional variant thereof) is operably linked to a chemically inducible promoter (as described herein); and/or the nucleic acid sequence encoding Rep78 (or a functional variant thereof) is operably linked to a chemically inducible promoter (as described herein); and/or the nucleic acid sequence encoding Rep68 (or a functional variant thereof) is operably linked to a chemically inducible promoter (as described herein); and/or the nucleic acid sequence encoding E2A (or a functional variant thereof) is operably linked to a chemically inducible promoter (as described herein); and/or the nucleic acid sequence encoding E4Orf6 (or a functional variant thereof) is operably linked to a chemically inducible promoter (as described herein); and/or the nucleic acid sequence encoding VARNA (or a functional variant thereof) is operably linked to a chemically inducible promoter (as described herein); and/or the nucleic acid sequence encoding Rep52 (or a functional variant thereof) is operably linked to a chemically inducible promoter (as described herein); and/or the nucleic acid sequence encoding VP2 (or a functional variant thereof) is operably linked to a chemically inducible promoter (as described herein); and/or the nucleic acid sequence encoding VP3 (or a functional variant thereof) is operably linked to a chemically inducible promoter (as described herein); and/or the nucleic acid sequence encoding AAP (or a functional variant thereof) is operably linked to a chemically inducible promoter (as described herein); and/or the nucleic acid sequence encoding MAAP (or a functional variant thereof) is operably linked to a chemically inducible promoter (as described herein).

In some embodiments, the nucleic acid sequence encoding VP2 (or a functional variant thereof) is operably linked to a chemically inducible promoter (as described herein); optionally wherein: the nucleic acid sequence encoding Rep40 (or a functional variant thereof) is operably linked to a chemically inducible promoter (as described herein); and/or the nucleic acid sequence encoding Rep78 (or a functional variant thereof) is operably linked to a chemically inducible promoter (as described herein); and/or the nucleic acid sequence encoding Rep68 (or a functional variant thereof) is operably linked to a chemically inducible promoter (as described herein); and/or the nucleic acid sequence encoding E2A (or a functional variant thereof) is operably linked to a chemically inducible promoter (as described herein); and/or the nucleic acid sequence encoding E4Orf6 (or a functional variant thereof) is operably linked to a chemically inducible promoter (as described herein); and/or the nucleic acid sequence encoding VARNA (or a functional variant thereof) is operably linked to a chemically inducible promoter (as described herein); and/or the nucleic acid sequence encoding VP1 (or a functional variant thereof) is operably linked to a chemically inducible promoter (as described herein); and/or the nucleic acid sequence encoding Rep52 (or a functional variant thereof) is operably linked to a chemically inducible promoter (as described herein); and/or the nucleic acid sequence encoding VP3 (or a functional variant thereof) is operably linked to a chemically inducible promoter (as described herein); and/or the nucleic acid sequence encoding AAP (or a functional variant thereof) is operably linked to a chemically inducible promoter (as described herein); and/or the nucleic acid sequence encoding MAAP (or a functional variant thereof) is operably linked to a chemically inducible promoter (as described herein).

In some embodiments, the nucleic acid sequence encoding VP3 (or a functional variant thereof) is operably linked to a chemically inducible promoter (as described herein); optionally wherein: the nucleic acid sequence encoding Rep40 (or a functional variant thereof) is operably linked to a chemically inducible promoter (as described herein); and/or the nucleic acid sequence encoding Rep78 (or a functional variant thereof) is operably linked to a chemically inducible promoter (as described herein); and/or the nucleic acid sequence encoding Rep68 (or a functional variant thereof) is operably linked to a chemically inducible promoter (as described herein); and/or the nucleic acid sequence encoding E2A (or a functional variant thereof) is operably linked to a chemically inducible promoter (as described herein); and/or the nucleic acid sequence encoding E4Orf6 (or a functional variant thereof) is operably linked to a chemically inducible promoter (as described herein); and/or the nucleic acid sequence encoding VARNA (or a functional variant thereof) is operably linked to a chemically inducible promoter (as described herein); and/or the nucleic acid sequence encoding VP1 (or a functional variant thereof) is operably linked to a chemically inducible promoter (as described herein); and/or the nucleic acid sequence encoding VP2 (or a functional variant thereof) is operably linked to a chemically inducible promoter (as described herein); and/or the nucleic acid sequence encoding Rep52 (or a functional variant thereof) is operably linked to a chemically inducible promoter (as described herein); and/or the nucleic acid sequence encoding AAP (or a functional variant thereof) is operably linked to a chemically inducible promoter (as described herein); and/or the nucleic acid sequence encoding MAAP (or a functional variant thereof) is operably linked to a chemically inducible promoter (as described herein).

In some embodiments, the nucleic acid sequence encoding AAP (or a functional variant thereof) is operably linked to a chemically inducible promoter (as described herein); optionally wherein: the nucleic acid sequence encoding Rep40 (or a functional variant thereof) is operably linked to a chemically inducible promoter (as described herein); and/or the nucleic acid sequence encoding Rep78 (or a functional variant thereof) is operably linked to a chemically inducible promoter (as described herein); and/or the nucleic acid sequence encoding Rep68 (or a functional variant thereof) is operably linked to a chemically inducible promoter (as described herein); and/or the nucleic acid sequence encoding E2A (or a functional variant thereof) is operably linked to a chemically inducible promoter (as described herein); and/or the nucleic acid sequence encoding E4Orf6 (or a functional variant thereof) is operably linked to a chemically inducible promoter (as described herein); and/or the nucleic acid sequence encoding VARNA (or a functional variant thereof) is operably linked to a chemically inducible promoter (as described herein); and/or the nucleic acid sequence encoding VP1 (or a functional variant thereof) is operably linked to a chemically inducible promoter (as described herein); and/or the nucleic acid sequence encoding VP2 (or a functional variant thereof) is operably linked to a chemically inducible promoter (as described herein); and/or the nucleic acid sequence encoding VP3 (or a functional variant thereof) is operably linked to a chemically inducible promoter (as described herein); and/or the nucleic acid sequence encoding Rep52 (or a functional variant thereof) is operably linked to a chemically inducible promoter (as described herein); and/or the nucleic acid sequence encoding MAAP (or a functional variant thereof) is operably linked to a chemically inducible promoter (as described herein).

In some embodiments, the nucleic acid sequence encoding MAAP (or a functional variant thereof) is operably linked to a chemically inducible promoter (as described herein); optionally wherein: the nucleic acid sequence encoding Rep40 (or a functional variant thereof) is operably linked to a chemically inducible promoter (as described herein); and/or the nucleic acid sequence encoding Rep78 (or a functional variant thereof) is operably linked to a chemically inducible promoter (as described herein); and/or the nucleic acid sequence encoding Rep68 (or a functional variant thereof) is operably linked to a chemically inducible promoter (as described herein); and/or the nucleic acid sequence encoding E2A (or a functional variant thereof) is operably linked to a chemically inducible promoter (as described herein); and/or the nucleic acid sequence encoding E4Orf6 (or a functional variant thereof) is operably linked to a chemically inducible promoter (as described herein); and/or the nucleic acid sequence encoding VARNA (or a functional variant thereof) is operably linked to a chemically inducible promoter (as described herein); and/or the nucleic acid sequence encoding VP1 (or a functional variant thereof) is operably linked to a chemically inducible promoter (as described herein); and/or the nucleic acid sequence encoding VP2 (or a functional variant thereof) is operably linked to a chemically inducible promoter (as described herein); and/or the nucleic acid sequence encoding VP3 (or a functional variant thereof) is operably linked to a chemically inducible promoter (as described herein); and/or the nucleic acid sequence encoding Rep52 (or a functional variant thereof) is operably linked to a chemically inducible promoter (as described herein); and/or the nucleic acid sequence encoding AAP (or a functional variant thereof) is operably linked to a chemically inducible promoter (as described herein).

In some embodiments, an AAV vector production system described herein comprises one or more polynucleotides that collectively encode for: Rep52 (or a functional variant thereof) or Rep40 (or a functional variant thereof); Rep78 (or a functional variant thereof) or Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); and AAP (or a functional variant thereof); each of which is operably linked to a chemically inducible promoter. In some embodiments, an AAV vector production system comprises one or more polynucleotides that collectively encode for: Rep52 (or a functional variant thereof); Rep40 (or a functional variant thereof); Rep78 (or a functional variant thereof); Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); AAP (or a functional variant thereof); and optionally MAAP (or a functional variant thereof); each of which is operably linked to a chemically inducible promoter.

In some embodiments, an AAV vector production system described herein comprises a single chemically inducible promoter. In such embodiments, the single chemically inducible promoter may be operably linked to a nucleic acid sequence encoding for: Rep52 (or a functional variant thereof) or Rep40 (or a functional variant thereof); Rep78 (or a functional variant thereof) or Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); and AAP (or a functional variant thereof). In some embodiments, the single chemically inducible promoter may be operably linked to a nucleic acid sequence encoding for: Rep52 (or a functional variant thereof); Rep40 (or a functional variant thereof); Rep78 (or a functional variant thereof); Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); AAP (or a functional variant thereof); and optionally MAAP (or a functional variant thereof).

In some embodiments, an AAV vector production system comprises at least two, at least three, at least four, or at least five chemically inducible promoters. In some embodiments, an AAV vector production system comprises 2, 3, 4, or 5 chemically inducible promoters.

In some embodiments, wherein an AAV vector production system comprises at least two chemically inducible promoters, two or more of the chemically inducible promoters comprise the same nucleic acid sequence. In some embodiments, wherein an AAV vector production system comprises at least two chemically inducible promoters, each of the chemically inducible promoters comprise the same nucleic acid sequence. In some embodiments, wherein an AAV vector production system comprises at least two chemically inducible promoters, one or more of the chemically inducible promoters comprises a distinct nucleic acid sequence. In some embodiments, wherein an AAV vector production system comprises at least two chemically inducible promoters, each of the chemically inducible promoters comprises a distinct nucleic acid sequence.

An inducible AAV vector production system described herein may further comprise a polynucleic acid comprising an expression cassette comprising; (i) a nucleic acid sequence of a promoter (constitutive or inducible, as described herein); and (ii) a nucleic acid sequence encoding a transcriptional activator, wherein the transcriptional activator, when expressed in the presence of a small molecule inducer, binds to a chemically inducible promoter of the engineered cell. In embodiments wherein an AAV vector production system comprises two or more distinct chemically inducible promoters, the system may comprise nucleic acid sequences of two or more corresponding transcriptional activators.

In some embodiments, a transcriptional activator is Tet-On 3G. In some embodiments, Tet-On 3G comprises the amino acid sequence of SEQ ID NO: 10. In some embodiments, a transcriptional activator is a functional variant of Tet-On 3G. A functional variant of Tet-On 3G comprises at least 80% identity (at least 85%, at least 90%, at least 95%, at least 96%, at least 97%, at least 98%, or at least 99% identity) with SEQ ID NO: 10 and maintains at least 80% of the function (at least 85%, at least 90%, at least 95%, at least 96%, at least 97%, at least 98%, or at least 99% of the function) of Tet-On 3G (that is, its function as a transcriptional activator).

In some embodiments, a transcriptional activator is TetOff-Advanced. In some embodiments, TetOff-Advanced comprises the amino acid sequence of SEQ ID NO: 11. In some embodiments, a transcriptional activator is a functional variant of TetOff-Advanced. A functional variant of TetOff-Advanced comprises at least 80% identity (at least 85%, at least 90%, at least 95%, at least 96%, at least 97%, at least 98%, or at least 99% identity) with SEQ ID NO: 11 and maintains at least 80% of the function (at least 85%, at least 90%, at least 95%, at least 96%, at least 97%, at least 98%, or at least 99% of the function) of TetOff-Advanced (that is, its function as a transcriptional activator).

In some embodiments, a transcriptional activator is VanR-VP16. In some embodiments, VanR-VP16 comprises the amino acid sequence of SEQ ID NO: 12. In some embodiments, a transcriptional activator is a functional variant of VanR-VP16. A functional variant of VanR-VP16 comprises at least 80% identity (at least 85%, at least 90%, at least 95%, at least 96%, at least 97%, at least 98%, or at least 99% identity) with SEQ ID NO: 12 and maintains at least 80% of the function (at least 85%, at least 90%, at least 95%, at least 96%, at least 97%, at least 98%, or at least 99% of the function) of VanR-VP16 (that is, its function as a transcriptional activator).

In some embodiments, a transcriptional activator is TtgR-VP16. In some embodiments, TtgR-VP16 comprises the amino acid sequence of SEQ ID NO: 13. In some embodiments, a transcriptional activator is a functional variant of TtgR-VP16. A functional variant of TtgR-VP16 comprises at least 80% identity (at least 85%, at least 90%, at least 95%, at least 96%, at least 97%, at least 98%, or at least 99% identity) with SEQ ID NO: 13 and maintains at least 80% of the function (at least 85%, at least 90%, at least 95%, at least 96%, at least 97%, at least 98%, or at least 99% of the function) of TtgR-VP16 (that is, its function as a transcriptional activator).

In some embodiments, a transcriptional activator is PhlF-VP16. In some embodiments, PhlF-VP16 comprises the amino acid sequence of SEQ ID NO: 14. In some embodiments, a transcriptional activator is a functional variant of PhlF-VP16. A functional variant of PhlF-VP16 comprises at least 80% identity (at least 85%, at least 90%, at least 95%, at least 96%, at least 97%, at least 98%, or at least 99% identity) with SEQ ID NO: 14 and maintains at least 80% of the function (at least 85%, at least 90%, at least 95%, at least 96%, at least 97%, at least 98%, or at least 99% of the function) of PhlF-VP16 (that is, its function as a transcriptional activator).

In some embodiments, a transcriptional activator is cTA. In some embodiments, cTA comprises the amino acid sequence of SEQ ID NO: 15. In some embodiments, a transcriptional activator is a functional variant of cTA. A functional variant of cTA comprises at least 80% identity (at least 85%, at least 90%, at least 95%, at least 96%, at least 97%, at least 98%, or at least 99% identity) with SEQ ID NO: 15 and maintains at least 80% of the function (at least 85%, at least 90%, at least 95%, at least 96%, at least 97%, at least 98%, or at least 99% of the function) of cTA (that is, its function as a transcriptional activator).

In some embodiments, a transcriptional activator is rcTA. In some embodiments, rcTA comprises the amino acid sequence of SEQ ID NO: 16. In some embodiments, a transcriptional activator is a functional variant of rcTA. A functional variant of rcTA comprises at least 80% identity (at least 85%, at least 90%, at least 95%, at least 96%, at least 97%, at least 98%, or at least 99% identity) with SEQ ID NO: 16 and maintains at least 80% of the function (at least 85%, at least 90%, at least 95%, at least 96%, at least 97%, at least 98%, or at least 99% of the function) of rcTA (that is, its function as a transcriptional activator).

### C. Exemplary AAV Vector Production System Architectures

For exemplary purpose, select AAV vector production system architectures are described below.

### 1. First Exemplary Architecture

In some embodiments, an AAV vector production system comprises: (a) a first polynucleic acid comprising at least one expression cassette; (b) a second polynucleic acid comprising at least two expression cassettes; and (c) a third expression cassette comprising at least two expression cassettes.

In some embodiments, the first polynucleic acid comprises: (i) a nucleic acid sequence encoding for Rep52 (or a functional variant thereof); a nucleic acid sequence encoding for Rep40 (or a functional variant thereof), or both; and (ii) a nucleic acid sequence encoding for Rep78 (or a functional variant thereof), a nucleic acid sequence encoding for Rep68 (or a functional variant thereof), or both. In some embodiments, the nucleic acid sequence encoding for (i) and the nucleic acid sequence encoding for (ii) are each operably linked to a first chemically inducible promoter (as described herein). In some embodiments, the first chemically inducible promoter comprises the nucleic acid sequence of one or more of SEQ ID NOs: 1-9. In some embodiments, the first polynucleic acid further comprises a nucleic acid sequence encoding for an internal ribosome entry site (IRES). In some embodiments, the nucleic acid sequence encoding for the IRES separates the nucleic acid sequence encoding for (i) and the nucleic acid sequence encoding for (ii). In some embodiments, the first polynucleic acid further comprises a selection marker that is operably linked to a promoter (constitutive or inducible, as described herein).

In some embodiments, the second polynucleic acid comprises the nucleic acid sequences encoding for E2A (or a functional variant thereof), E4Orf6 (or a functional variant thereof), and VARNA (or a functional variant thereof). In some embodiments, the nucleic acid sequences encoding for E2A (or a functional variant thereof), E4Orf6 (or a functional variant thereof), and VARNA (or a functional variant thereof) are each operably linked to a second chemically inducible promoter (as described herein). In some embodiments, the second chemically inducible promoter comprises the nucleic acid sequence of one or more of SEQ ID NOs: 1-9. In some embodiments, the second polynucleic acid further comprises a nucleic acid sequence encoding an IRES. In some embodiments, the nucleic acid sequence encoding for the IRES separates the nucleic acid sequence encoding for E2A (or a functional variant thereof) and the nucleic acid sequence encoding for E4Orf6 (or a functional variant thereof). In some embodiments, the second polynucleic acid further comprises a selection marker that is operably linked to a promoter (constitutive or inducible, as described herein).

In some embodiments, the third stably integrated polynucleic acid comprises the nucleic acid sequences encoding for VP1 (or a functional variant thereof), VP2 (or a functional variant thereof), VP3 (or a functional variant thereof), AAP (or a functional variant thereof); and optionally MAAP (or a functional variant thereof). In some embodiments, the third polynucleic acid further comprises a selection marker that is operably linked to a promoter. In some embodiments, the nucleic acid sequences encoding for VP1 (or a functional variant thereof), VP2 (or a functional variant thereof), VP3 (or a functional variant thereof), and AAP (or a functional variant thereof) are each operably linked to a third chemically inducible promoter (as described herein). In some embodiments, the third chemically inducible promoter comprises the nucleic acid sequence of one or more of SEQ ID NOs: 1-9.

In some embodiments, the AAV vector production system comprises a fourth polynucleic acid, wherein the fourth polynucleic acid comprises a nucleic acid sequence encoding for a transcriptional activator that, when expressed in the presence of a small molecule inducer, binds to a chemically inducible promoter of the AAV production system. In some embodiments, the transcriptional activator comprises the amino acid sequence of any one of SEQ ID NOs: 10-16. In some embodiments, the fourth polynucleic acid further comprises a selection marker that is operably linked to a promoter (constitutive or inducible, as described herein).

### 2. Second Exemplary Architecture

In some embodiments, an AAV vector production system comprises one or more stably integrated polynucleic acid collectively comprising a nucleic acid sequence encoding for each of: Rep52 (or a functional variant thereof) or Rep40 (or a functional variant thereof); Rep78 (or a functional variant thereof) or Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); AAP (or a functional variant thereof); and optionally MAAP (or a functional variant thereof); at least one of which is operably linked to a chemically inducible promoter (as described herein); wherein: (i) the nucleic acid sequence encoding for Rep68 (or a functional variant thereof) lacks the Rep52 start codon: (ii) the nucleic acid sequence encoding for Rep78 (or a functional variant thereof) lacks the Rep52 start codon and the Rep68/40 splice site; (iii) the nucleic acid sequence encoding for Rep52 (or a functional variant thereof) lacks the Rep40 splice site; and (iv) the nucleic acid sequence encoding for VP1 (or a functional variant thereof) lacks start codons for VP2, VP3, and AAP.

In some embodiments, a first polynucleic acid comprises: (i) a nucleic acid sequence encoding for Rep52 (or a functional variant thereof); a nucleic acid sequence encoding for Rep40 (or a functional variant thereof), or both; and (ii) a nucleic acid sequence encoding for Rep78 (or a functional variant thereof), a nucleic acid sequence encoding for Rep68 (or a functional variant thereof), or both. In some embodiments, the first polynucleic acid comprises the nucleic acid sequence encoding for Rep52 (or a functional variant thereof) and the nucleic acid sequence encoding for Rep78 (or a functional variant thereof) or Rep68 (or a functional variant thereof). In some embodiments, the nucleic acid sequence encoding for Rep52 (or a functional variant thereof) and the nucleic acid sequence encoding for Rep78 (or a functional variant thereof) or Rep68 (or a functional variant thereof) are each operably linked to a first chemically inducible promoter (as described herein). In some embodiments, the first chemically inducible promoter comprises the nucleic acid sequence of one or more of SEQ ID NOs: 1-9. In some embodiments, the first polynucleic acid further comprises a nucleic acid sequence encoding for an internal ribosome entry site (IRES) or a 2A peptide. In some embodiments, the nucleic acid sequence encoding for the IRES or the 2A peptide separates the nucleic acid sequence encoding for Rep52 (or a functional variant thereof) and the nucleic acid sequence encoding for Rep78 (or a functional variant thereof) or Rep68 (or a functional variant thereof). In some embodiments, the first polynucleic acid further comprises a selection marker that is operably linked to a promoter (constitutive or inducible, as described herein).

In some embodiments, a second polynucleic acid comprises the nucleic acid sequence encoding for E2A (or a functional variant thereof), the nucleic acid sequence encoding for E4Orf6 (or a functional variant thereof), and the nucleic acid sequence encoding for VARNA (or a functional variant thereof). In some embodiments, the nucleic acid sequence encoding for E2A (or a functional variant thereof), the nucleic acid sequence encoding for E4Orf6 (or a functional variant thereof), and the nucleic acid sequence encoding for VARNA (or a functional variant thereof) are each operably linked to the second chemically inducible promoter (as described herein). In some embodiments, the second chemically inducible promoter comprises the nucleic acid sequence of one or more of SEQ ID NOs: 1-9. In some embodiments, the second polynucleic acid further comprises a nucleic acid sequence encoding for an internal ribosome entry site (IRES) or a 2A peptide. In some embodiments, the nucleic acid sequence encoding for the IRES or the 2A peptide separates the nucleic acid sequence encoding for E2A and the nucleic acid sequence encoding E4Orf6. In some embodiments, the second polynucleic acid further comprises a selection marker that is operably linked to a promoter (constitutive or inducible, as described herein).

In some embodiments, a third polynucleic acid comprises the nucleic acid sequence encoding for VP1 (or a functional variant thereof), the nucleic acid sequence encoding for VP2 (or a functional variant thereof), the nucleic acid sequence encoding for VP3 (or a functional variant thereof), the nucleic acid sequence encoding for AAP (or a functional variant thereof), and optionally the nucleic acid sequence encoding for MAAP (or a functional variant thereof). In some embodiments, the nucleic acid sequence encoding for VP1 (or a functional variant thereof) is operably linked to a third chemically inducible promoter (as described herein) and the nucleic acid sequence encoding for VP2 (or a functional variant thereof), the nucleic acid sequence encoding for VP3 (or a functional variant thereof), and the nucleic acid sequence encoding for AAP (or a functional variant thereof) are each operably linked to a fourth chemically inducible promoter (as described herein). In some embodiments, the third chemically inducible promoter comprises the nucleic acid sequence of one or more of SEQ ID NOs: 1-9. In some embodiments, the fourth chemically inducible promoter comprises the nucleic acid sequence of one or more of SEQ ID NOs: 1-9. In some embodiments, the third polynucleic acid further comprises a selection marker that is operably linked to a promoter (constitutive or inducible, as described herein).

In some embodiments, an AAV vector production system comprises a fourth polynucleic acid, wherein the fourth polynucleic acid comprises a nucleic acid sequence encoding for a transcriptional activator that, when expressed in the presence of a small molecule inducer, binds to a chemically inducible promoter of the engineered cell. In some embodiments, the transcriptional activator comprises the amino acid sequence of any one of SEQ ID NOs: 10-16. In some embodiments, the fourth polynucleic molecule further comprises a selection marker that is operably linked to a promoter (constitutive or inducible, as described herein).

### 3. Third Exemplary Architecture

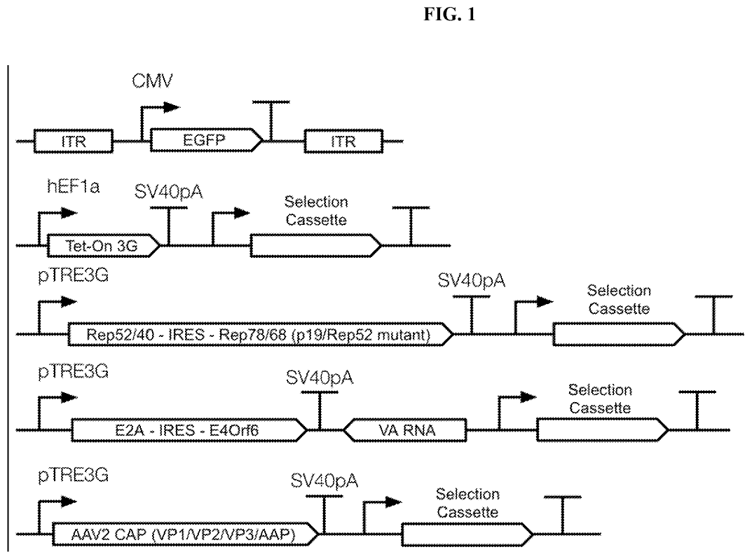

In some embodiments, an AAV vector production system architecture is as depicted in FIG. 1.

### 4. Fourth Exemplary Architecture

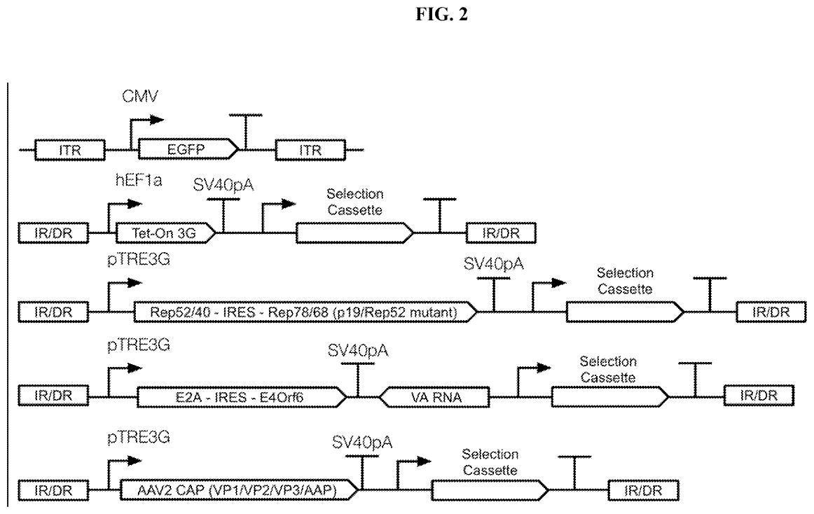

In some embodiments, an AAV vector production system architecture is as depicted in FIG. 2.

### 5. Fifth Exemplary Architecture

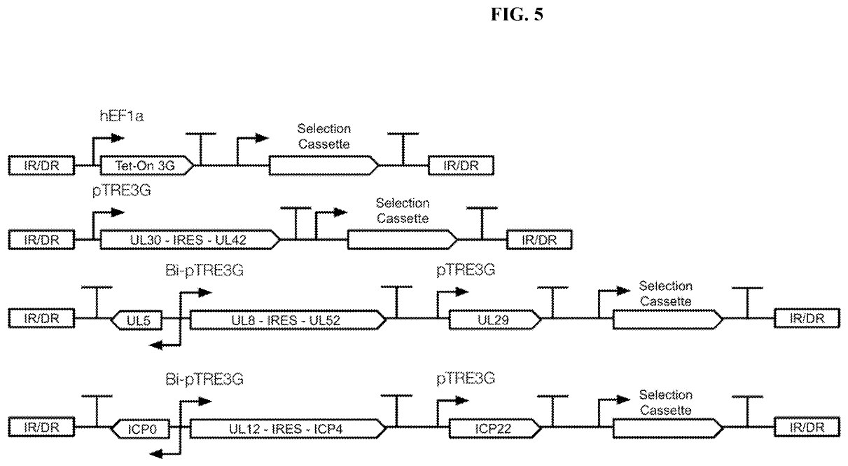

In some embodiments, an AAV vector production system architecture is as depicted in FIG. 5.

### II. HSV-Helper Systems for AAV Vector Production

In some aspects, the disclosure relates to HSV-helper Systems for AAV vector production. A helper system, as described herein, comprises one or more polynucleic acids collectively encoding for UL5 (or a functional variant thereof), UL8 (or a functional variant thereof), UL29 (or a functional variant thereof), UL30 (or a functional variant thereof), UL42 (or a functional variant thereof), UL52 (or a functional variant thereof), UL12 (or a functional variant thereof), ICP10 (or a functional variant thereof), ICP4 (or a functional variant thereof), and ICP22 (or a functional variant thereof).

In some embodiments, a helper system is encoded on a single polynucleic acid. In other embodiments, multiple polynucleic acids collectively comprise a helper system. For example, a helper system may comprise at least 2, at least 3, at least 4, or at least 5 polynucleic acids. In some embodiments, a helper system comprises 2, 3, 4, or 5 polynucleic acids. Exemplary helper system architectures are provided below (Part IC).

UL5 (together with UL8 and UL52) is part of a helicase/primase complex that aids AAV genome replication. In some embodiments, a helper system comprises a polynucleic acid encoding for UL5. In some embodiments, UL5 comprises the amino acid sequence of SEQ ID NO: 41. In some embodiments, a helper system comprises a polynucleic acid encoding for a functional variant of UL5. A functional variant of UL5 comprises at least 80% identity (at least 85%, at least 90%, at least 95%, at least 96%, at least 97%, at least 98%, or at least 99% identity) with SEQ ID NO: 41 and maintains at least 80% of the function (at least 85%, at least 90%, at least 95%, at least 96%, at least 97%, at least 98%, or at least 99% of the function) of UL5 (that is, its function in AAV genome replication).

UL8 (together with UL5 and UL52) is part of a helicase/primase complex that aids AAV genome replication. In some embodiments, a helper system comprises a polynucleic acid encoding for UL8. In some embodiments, UL8 comprises the amino acid sequence of SEQ ID NO: 42. In some embodiments, a helper system comprises a polynucleic acid encoding for a functional variant of UL8. A functional variant of UL8 comprises at least 80% identity (at least 85%, at least 90%, at least 95%, at least 96%, at least 97%, at least 98%, or at least 99% identity) with SEQ ID NO: 42 and maintains at least 80% of the function (at least 85%, at least 90%, at least 95%, at least 96%, at least 97%, at least 98%, or at least 99% of the function) of UL8 (that is, its function in AAV genome replication).

UL52 (together with UL5 and UL8) is part of a helicase/primase complex that aids AAV genome replication. In some embodiments, a helper system comprises a polynucleic acid encoding for UL52. In some embodiments, UL52 comprises the amino acid sequence of SEQ ID NO: 43. In some embodiments, a helper system comprises a polynucleic acid encoding for a functional variant of UL52. A functional variant of UL52 comprises at least 80% identity (at least 85%, at least 90%, at least 95%, at least 96%, at least 97%, at least 98%, or at least 99% identity) with SEQ ID NO: 43 and maintains at least 80% of the function (at least 85%, at least 90%, at least 95%, at least 96%, at least 97%, at least 98%, or at least 99% of the function) of UL52 (that is, its function in AAV genome replication).

UL30 (together with its cofactor UL42) is part of a polymerase complex that aids AAV genome replication. In some embodiments, a helper system comprises a polynucleic acid encoding for UL30. In some embodiments, UL30 comprises the amino acid sequence of SEQ ID NO: 39. In some embodiments, a helper system comprises a polynucleic acid encoding for a functional variant of UL30. A functional variant of UL30 comprises at least 80% identity (at least 85%, at least 90%, at least 95%, at least 96%, at least 97%, at least 98%, or at least 99% identity) with SEQ ID NO: 39 and maintains at least 80% of the function (at least 85%, at least 90%, at least 95%, at least 96%, at least 97%, at least 98%, or at least 99% of the function) of UL30 (that is, its function in AAV genome replication).

UL42 (together with UL30) is part of a polymerase complex that aids AAV genome replication. In some embodiments, a helper system comprises a polynucleic acid encoding for UL42. In some embodiments, UL42 comprises the amino acid sequence of SEQ ID NO: 40. In some embodiments, a helper system comprises a polynucleic acid encoding for a functional variant of UL42. A functional variant of UL42 comprises at least 80% identity (at least 85%, at least 90%, at least 95%, at least 96%, at least 97%, at least 98%, or at least 99% identity) with SEQ ID NO: 40 and maintains at least 80% of the function (at least 85%, at least 90%, at least 95%, at least 96%, at least 97%, at least 98%, or at least 99% of the function) of UL42 (that is, its function in AAV genome replication).

UL29 (or ICP8) is a DNA binding protein that aids AAV genome replication. In some embodiments, a helper system comprises a polynucleic acid encoding for UL29. In some embodiments, UL29 comprises the amino acid sequence of SEQ ID NO: 44. In some embodiments, a helper system comprises a polynucleic acid encoding for a functional variant of UL29. A functional variant of UL29 comprises at least 80% identity (at least 85%, at least 90%, at least 95%, at least 96%, at least 97%, at least 98%, or at least 99% identity) with SEQ ID NO: 44 and maintains at least 80% of the function (at least 85%, at least 90%, at least 95%, at least 96%, at least 97%, at least 98%, or at least 99% of the function) of UL29 (that is, its function in AAV genome replication).

UL12 is a nuclease that plays a role in replication (Reuven et al. Journal of virology 78.9 (2004): 4599-4608, which is incorporated by reference in its entirety). In some embodiments, a helper system comprises a polynucleic acid encoding for UL12. In some embodiments, UL12 comprises the amino acid sequence of SEQ ID NO: 50. In some embodiments, a helper system comprises a polynucleic acid encoding for a functional variant of UL12. A functional variant of UL12 comprises at least 80% identity (at least 85%, at least 90%, at least 95%, at least 96%, at least 97%, at least 98%, or at least 99% identity) with SEQ ID NO: 50 and maintains at least 80% of the function (at least 85%, at least 90%, at least 95%, at least 96%, at least 97%, at least 98%, or at least 99% of the function) of UL12 (that is, its function in AAV genome replication).

ICP0 is a RING protein that has E3 ubiquitin ligase activity which plays a role in balancing lytic replication and latency (Smith et al. Future virology 6.4 (2011): 421-429, which is incorporated by reference in its entirety). In some embodiments, a helper system comprises a polynucleic acid encoding for ICP0. In some embodiments, ICP0 comprises the amino acid sequence of SEQ ID NO: 51. In some embodiments, a helper system comprises a polynucleic acid encoding for a functional variant of ICP0. A functional variant of ICP0 comprises at least 80% identity (at least 85%, at least 90%, at least 95%, at least 96%, at least 97%, at least 98%, or at least 99% identity) with SEQ ID NO: 51 and maintains at least 80% of the function (at least 85%, at least 90%, at least 95%, at least 96%, at least 97%, at least 98%, or at least 99% of the function) of ICP0 (that is, its function as a ubiquitin ligase).

ICP4 is a 1298 amino acid nuclear phosphoprotein required to activate the transcription of the early and late genes (11, 37). Consistent with its ability to bind to viral DNA (3, 33), ICP4 is recruited into viral replication compartments containing large amounts of replicating viral DNA. In some embodiments, a helper system comprises a polynucleic acid encoding for ICP4. In some embodiments, ICP4 comprises the amino acid sequence of SEQ ID NO: 52. In some embodiments, a helper system comprises a polynucleic acid encoding for a functional variant of ICP4. A functional variant of ICP4 comprises at least 80% identity (at least 85%, at least 90%, at least 95%, at least 96%, at least 97%, at least 98%, or at least 99% identity) with SEQ ID NO: 52 and maintains at least 80% of the function (at least 85%, at least 90%, at least 95%, at least 96%, at least 97%, at least 98%, or at least 99% of the function) of ICP4 (that is, its function in AAV viral growth).

ICP22 regulates viral genes associated with viral primary envelopment (Maruzuru, Yuhei, et al. Journal of virology 88.13 (2014): 7445-7454). In some embodiments, a helper system comprises a polynucleic acid encoding for ICP22. In some embodiments, ICP22 comprises the amino acid sequence of SEQ ID NO: 53. In some embodiments, a helper system comprises a polynucleic acid encoding for a functional variant of ICP22. A functional variant of ICP22 comprises at least 80% identity (at least 85%, at least 90%, at least 95%, at least 96%, at least 97%, at least 98%, or at least 99% identity) with SEQ ID NO: 53 and maintains at least 80% of the function (at least 85%, at least 90%, at least 95%, at least 96%, at least 97%, at least 98%, or at least 99% of the function) of ICP22 (that is, its function in AAV regulating genes associated with primary envelopment).

The helper systems described herein comprise at least one expression cassette. As used herein, the term “expression cassette” refers to a polynucleic acid sequence encoding a nucleic acid sequence of a promoter that is operably linked to a nucleic acid encoding a product (e.g., an RNA product(s) and/or a polypeptide product(s), such as UL5 (or a functional variant thereof), UL8 (or a functional variant thereof), UL29 (or a functional variant thereof), UL30 (or a functional variant thereof), UL42 (or a functional variant thereof), UL52 (or a functional variant thereof), ICP0 (or a functional variant thereof), UL12 (or a functional variant thereof), ICP4 (or a functional variant thereof), ICP22 (or a functional variant thereof), or any combination thereof). In some embodiments, multiple products are encoded within a single expression cassette. For example, in some embodiments, a single promoter drives expression of a polycistronic RNA encoding for multiple products (an RNA product(s) and/or a polypeptide product(s)). A polycistronic RNA may comprise a nucleic acid sequence of an internal ribosomal entry site (IRES) and/or a nucleic acid sequence of a viral 2A peptide (V2A).

An IRES may comprises the nucleic acid sequence of SEQ ID NO: 45:

CCCCTCTCCCTCCCCCCCCCCTAACGTTACTGGCCGAAGCCGCTTGGAA
TAAGGCCGGTGTGCGTTTGTCTATATGTTATTTTCCACCATATTGCCGT
CTTTTGGCAATGTGAGGGCCCGGAAACCTGGCCCTGTCTTCTTGACGAG
CATTCCTAGGGGTCTTTCCCCTCTCGCCAAAGGAATGCAAGGTCTGTTG
AATGTCGTGAAGGAAGCAGTTCCTCTGGAAGCTTCTTGAAGACAAACAA
CGTCTGTAGCGACCCTTTGCAGGCAGCGGAACCCCCCACCTGGCGACAG
GTGCCTCTGCGGCCAAAAGCCACGTGTATAAGATACACCTGCAAAGGCG
GCACAACCCCAGTGCCACGTTGTGAGTTGGATAGTTGTGGAAAGAGTCA
AATGGCTCTCCTCAAGCGTATTCAACAAGGGGCTGAAGGATGCCCAGAA
GGTACCCCATTGTATGGGATCTGATCTGGGGCCTCGGTGCACATGCTTT
ACATGTGTTTAGTCGAGGTTAAAAAAACGTCTAGGCCCCCCGAACCACG
GGGACGTGGTTTTCCTTTGAAAAACACGATGATAATATG

An IRES may comprise the nucleic acid sequence of SE ID NO: 46:

CCCCCCCCCCTAACGTTACTGGCCGAAGCCGCTTGGAATAAGGCCGGTG
TGCGTTTGTCTATATGTTATTTTCCACCATATTGCCGTCTTTTGGCAAT
GTGAGGGCCCGGAAACCTGGCCCTGTCTTCTTGACGAGCATTCCTAGGG
GTCTTTCCCCTCTCGCCAAAGGAATGCAAGGTCTGTTGAATGTCGTGAA
GGAAGCAGTTCCTCTGGAAGCTTCTTGAAGACAAACAACGTCTGTAGCG
ACCCTTTGCAGGCAGCGGAACCCCCCACCTGGCGACAGGTGCCTCTGCG
GCCAAAAGCCACGTGTATAAGATACACCTGCAAAGGCGGCACAACCCCA
GTGCCACGTTGTGAGTTGGATAGTTGTGGAAAGAGTCAAATGGCTCTCC
TCAAGCGTATTCAACAAGGGGCTGAAGGATGCCCAGAAGGTACCCCATT
GTATGGGATCTGATCTGGGGCCTCGGTGCACATGCTTTACATGTGTTTA
GTCGAGGTTAAAAAAACGTCTAGGCCCCCCGAACCACGGGGACGTGGTT
TTCCTTTGAAAAACACGATGATAATAGTTATC

A viral 2A peptide may comprise the amino acid sequence of

(SEQ ID NO: 47)
ATNFSLLKQAGDVEENPGP
or
(SEQ ID NO: 48)
EGRGSLLTCGDVEENPGP.

In some embodiments, a helper system comprises multiple cassettes. In instances when a helper system comprises multiple cassettes, the cassettes may be positioned in various orientations. For example, in some embodiments, each of the cassettes are encoded in the same orientation (i.e., encoding on the same strand). In other embodiments, at least one cassette is encoded in an opposite orientation (i.e., encoding on the opposite strand). Cassettes positioned in an opposite orientation may have convergent expression (→←) or divergent expression (←→). In some embodiments, the expression cassettes are positioned in an alternating orientation.

As described herein, a promoter is “operably linked” to a nucleic acid coding sequence when the position of the promoter relative to the nucleic acid coding sequence is such that binding of a transcriptional activator to the promoter can induce expression of the coding sequence. A promoter of an expression cassette may be a constitutive promoter or an inducible promoter.

A promoter may be a constitutive promoter (i.e., an unregulated promoter that allows for continual transcription). Examples of constitutive promoters are known in the art and include, but are not limited to, cytomegalovirus (CMV) promoters, elongation factor 1α (EF1α) promoters, simian vacuolating virus 40 (SV40) promoters, ubiquitin-C (UBC) promoters, U6 promoters, and phosphoglycerate kinase (PGK) promoters. See e.g., Ferreira et al., Tuning gene expression with synthetic upstream open reading frames. Proc. Natl. Acad. Sci. U.S.A. 2013 July; 110(28): 11284-89; Pub. No.: US 2014/377861 A1—the entireties of which are incorporated herein by reference.

Alternatively, a promoter may be an inducible promoter (i.e., only activates transcription under specific circumstances). An inducible promoter may be a chemically inducible promoter, a temperature inducible promoter, or a light inducible promoter. Examples of chemically inducible promoters are known in the art and include, but are not limited to, tetracycline/doxycycline inducible promoters, cumate inducible promoters, ABA inducible promoters, CRY2-CIB1 inducible promoters, DAPG inducible promoters, and mifepristone inducible promoters. See e.g., Stanton et al., ACS Synth. Biol. 2014 Dec. 19; 3(12): 880-91; Liang et al., Sci. Signal. 2011 Mar. 15; 4(164): rs2; U.S. Pat. No. 7,745,592 B2; U.S. Pat. No. 7,935,788 B2—the entireties of which are incorporated herein by reference. A chemically inducible promoter may comprise the nucleic acid sequence of one or more of SEQ ID NOs: 1-9.

### A. Selectable Markers

As described above, a helper system, as described herein, comprises one or more polynucleic acids that collectively encode for: UL5 (or a functional variant thereof), UL8 (or a functional variant thereof), UL29 (or a functional variant thereof), UL30 (or a functional variant thereof), UL42 (or a functional variant thereof), and UL52 (or a functional variant thereof). In some embodiments, one or more of the polynucleic acids of a helper system comprises an expression cassette comprising: (i) a nucleic acid sequence of a promoter (constitutive or inducible, as described herein); and (ii) a nucleic acid sequence encoding a selectable maker. In some embodiments, each of the polynucleic acids of a helper system comprises a selectable maker. In some embodiments, each polynucleic acid of a helper system comprises a nucleic acid sequence of a distinct selectable marker.

As used herein, the term “selectable marker” refers to a protein that—when introduced into or expressed in a cell—confers a trait that is suitable for selection.

A selectable marker may be a fluorescent protein. Examples of fluorescent proteins are known in the art (e.g., TagBFP, EBFP2, EGFP, EYFP, mKO2, or Sirius). See e.g., U.S. Pat. No. 5,874,304; Patent No.: EP 0969284 A1; Pub. No.: US 2010/167394 A—the entireties of which are incorporated here by reference.

Alternatively, or in addition, a selectable marker may be an antibiotic resistance protein. Examples of antibiotic resistance proteins are known in the art (e.g., facilitating puromycin, hygromycin, Geneticin™ (G418), neomycin, zeocin, blasticidin, or phleomycin selection). See e.g., Pub. No.: WO 1997/15668 A2; Pub. No.: WO 1997/43900 A1—the entireties of which are incorporated here by reference.

### B. Inducible HSV-Helper Systems

In some embodiments, an HSV-helper system described herein comprises one or more polynucleotides that collectively encode for: UL5 (or a functional variant thereof); UL8 (or a functional variant thereof); UL29 (or a functional variant thereof); UL30 (or a functional variant thereof); UL42 (or a functional variant thereof); and UL52 (or a functional variant thereof); at least one of which is operably linked to a chemically inducible promoter. In some embodiments, the one or more polynucleotides of an HSV-helper system described herein further encode for (collectively): ICP0 (or a functional variant thereof); UL12 (or a functional variant thereof); ICP4 (or a functional variant thereof); and ICP22 (or a functional variant thereof). In some embodiments, the sequence encoding for ICP0 (or a functional variant thereof); UL12 (or a functional variant thereof); ICP4 (or a functional variant thereof); and/or ICP22 (or a functional variant thereof) is operably linked to a chemically inducible promoter.

In any of the embodiments described in this section, a chemically inducible promoter may comprise a tetracycline/doxycycline inducible promoter, a cumate inducible promoter, a ABA inducible promoter, a CRY2-CIB1 inducible promoter, a DAPG inducible promoter, a mifepristone inducible promoter, or a combination thereof. In some embodiments, a chemically inducible promoter comprises the nucleic acid sequence of one or more of SEQ ID NOs: 1-9. In some embodiments, a chemically inducible promoter comprises the nucleic acid sequence of SEQ ID NO: 1. In some embodiments, a chemically inducible promoter comprises the nucleic acid sequence of SEQ ID NO: 2. In some embodiments, a chemically inducible promoter comprises the nucleic acid sequence of SEQ ID NO: 3. In some embodiments, a chemically inducible promoter comprises the nucleic acid sequence of SEQ ID NO: 4. In some embodiments, a chemically inducible promoter comprises the nucleic acid sequence of SEQ ID NO: 5. In some embodiments, a chemically inducible promoter comprises the nucleic acid sequence of SEQ ID NO: 6. In some embodiments, a chemically inducible promoter comprises the nucleic acid sequence of SEQ ID NO: 7. In some embodiments, a chemically inducible promoter comprises the nucleic acid sequence of SEQ ID NO: 8. In some embodiments, a chemically inducible promoter comprises the nucleic acid sequence of SEQ ID NO: 9.

In some embodiments, the nucleic acid sequence encoding UL5 (or a functional variant thereof) is operably linked to a chemically inducible promoter (as described herein); optionally wherein: the nucleic acid sequence encoding UL8 (or a functional variant thereof) is operably linked to a chemically inducible promoter (as described herein); and/or the nucleic acid sequence encoding UL29 (or a functional variant thereof) is operably linked to a chemically inducible promoter (as described herein); and/or the nucleic acid sequence encoding UL30 (or a functional variant thereof) is operably linked to a chemically inducible promoter (as described herein); and/or the nucleic acid sequence encoding UL42 (or a functional variant thereof) is operably linked to a chemically inducible promoter (as described herein); and/or the nucleic acid sequence encoding UL52 (or a functional variant thereof) is operably linked to a chemically inducible promoter (as described herein); and/or the nucleic acid sequence encoding ICP0 (or a functional variant thereof) is operably linked to a chemically inducible promoter (as described herein); and/or the nucleic acid sequence encoding UL12 (or a functional variant thereof) is operably linked to a chemically inducible promoter (as described herein); and/or the nucleic acid sequence encoding ICP4 (or a functional variant thereof) is operably linked to a chemically inducible promoter (as described herein); and/or the nucleic acid sequence encoding ICP22 (or a functional variant thereof) is operably linked to a chemically inducible promoter (as described herein).

In some embodiments, the nucleic acid sequence encoding UL8 (or a functional variant thereof) is operably linked to a chemically inducible promoter (as described herein); optionally wherein: the nucleic acid sequence encoding UL5 (or a functional variant thereof) is operably linked to a chemically inducible promoter (as described herein); and/or the nucleic acid sequence encoding UL29 (or a functional variant thereof) is operably linked to a chemically inducible promoter (as described herein); and/or the nucleic acid sequence encoding UL30 (or a functional variant thereof) is operably linked to a chemically inducible promoter (as described herein); and/or the nucleic acid sequence encoding UL42 (or a functional variant thereof) is operably linked to a chemically inducible promoter (as described herein); and/or the nucleic acid sequence encoding UL52 (or a functional variant thereof) is operably linked to a chemically inducible promoter (as described herein); and/or the nucleic acid sequence encoding ICP0 (or a functional variant thereof) is operably linked to a chemically inducible promoter (as described herein); and/or the nucleic acid sequence encoding UL12 (or a functional variant thereof) is operably linked to a chemically inducible promoter (as described herein); and/or the nucleic acid sequence encoding ICP4 (or a functional variant thereof) is operably linked to a chemically inducible promoter (as described herein); and/or the nucleic acid sequence encoding ICP22 (or a functional variant thereof) is operably linked to a chemically inducible promoter (as described herein).

In some embodiments, the nucleic acid sequence encoding UL29 (or a functional variant thereof) is operably linked to a chemically inducible promoter (as described herein); optionally wherein: the nucleic acid sequence encoding UL8 (or a functional variant thereof) is operably linked to a chemically inducible promoter (as described herein); and/or the nucleic acid sequence encoding UL5 (or a functional variant thereof) is operably linked to a chemically inducible promoter (as described herein); and/or the nucleic acid sequence encoding UL30 (or a functional variant thereof) is operably linked to a chemically inducible promoter (as described herein); and/or the nucleic acid sequence encoding UL42 (or a functional variant thereof) is operably linked to a chemically inducible promoter (as described herein); and/or the nucleic acid sequence encoding UL52 (or a functional variant thereof) is operably linked to a chemically inducible promoter (as described herein); and/or the nucleic acid sequence encoding ICP0 (or a functional variant thereof) is operably linked to a chemically inducible promoter (as described herein); and/or the nucleic acid sequence encoding UL12 (or a functional variant thereof) is operably linked to a chemically inducible promoter (as described herein); and/or the nucleic acid sequence encoding ICP4 (or a functional variant thereof) is operably linked to a chemically inducible promoter (as described herein); and/or the nucleic acid sequence encoding ICP22 (or a functional variant thereof) is operably linked to a chemically inducible promoter (as described herein).

In some embodiments, the nucleic acid sequence encoding UL30 (or a functional variant thereof) is operably linked to a chemically inducible promoter (as described herein); optionally wherein: the nucleic acid sequence encoding UL8 (or a functional variant thereof) is operably linked to a chemically inducible promoter (as described herein); and/or the nucleic acid sequence encoding UL29 (or a functional variant thereof) is operably linked to a chemically inducible promoter (as described herein); and/or the nucleic acid sequence encoding UL5 (or a functional variant thereof) is operably linked to a chemically inducible promoter (as described herein); and/or the nucleic acid sequence encoding UL42 (or a functional variant thereof) is operably linked to a chemically inducible promoter (as described herein); and/or the nucleic acid sequence encoding UL52 (or a functional variant thereof) is operably linked to a chemically inducible promoter (as described herein); and/or the nucleic acid sequence encoding ICP0 (or a functional variant thereof) is operably linked to a chemically inducible promoter (as described herein); and/or the nucleic acid sequence encoding UL12 (or a functional variant thereof) is operably linked to a chemically inducible promoter (as described herein); and/or the nucleic acid sequence encoding ICP4 (or a functional variant thereof) is operably linked to a chemically inducible promoter (as described herein); and/or the nucleic acid sequence encoding ICP22 (or a functional variant thereof) is operably linked to a chemically inducible promoter (as described herein).

In some embodiments, the nucleic acid sequence encoding UL42 (or a functional variant thereof) is operably linked to a chemically inducible promoter (as described herein); optionally wherein: the nucleic acid sequence encoding UL8 (or a functional variant thereof) is operably linked to a chemically inducible promoter (as described herein); and/or the nucleic acid sequence encoding UL29 (or a functional variant thereof) is operably linked to a chemically inducible promoter (as described herein); and/or the nucleic acid sequence encoding UL30 (or a functional variant thereof) is operably linked to a chemically inducible promoter (as described herein); and/or the nucleic acid sequence encoding UL5 (or a functional variant thereof) is operably linked to a chemically inducible promoter (as described herein); and/or the nucleic acid sequence encoding UL52 (or a functional variant thereof) is operably linked to a chemically inducible promoter (as described herein); and/or the nucleic acid sequence encoding ICP0 (or a functional variant thereof) is operably linked to a chemically inducible promoter (as described herein); and/or the nucleic acid sequence encoding UL12 (or a functional variant thereof) is operably linked to a chemically inducible promoter (as described herein); and/or the nucleic acid sequence encoding ICP4 (or a functional variant thereof) is operably linked to a chemically inducible promoter (as described herein); and/or the nucleic acid sequence encoding ICP22 (or a functional variant thereof) is operably linked to a chemically inducible promoter (as described herein).

In some embodiments, the nucleic acid sequence encoding UL52 (or a functional variant thereof) is operably linked to a chemically inducible promoter (as described herein); optionally wherein: the nucleic acid sequence encoding UL8 (or a functional variant thereof) is operably linked to a chemically inducible promoter (as described herein); and/or the nucleic acid sequence encoding UL29 (or a functional variant thereof) is operably linked to a chemically inducible promoter (as described herein); and/or the nucleic acid sequence encoding UL30 (or a functional variant thereof) is operably linked to a chemically inducible promoter (as described herein); and/or the nucleic acid sequence encoding UL42 (or a functional variant thereof) is operably linked to a chemically inducible promoter (as described herein); and/or the nucleic acid sequence encoding UL5 (or a functional variant thereof) is operably linked to a chemically inducible promoter (as described herein); and/or the nucleic acid sequence encoding ICP0 (or a functional variant thereof) is operably linked to a chemically inducible promoter (as described herein); and/or the nucleic acid sequence encoding UL12 (or a functional variant thereof) is operably linked to a chemically inducible promoter (as described herein); and/or the nucleic acid sequence encoding ICP4 (or a functional variant thereof) is operably linked to a chemically inducible promoter (as described herein); and/or the nucleic acid sequence encoding ICP22 (or a functional variant thereof) is operably linked to a chemically inducible promoter (as described herein).

In some embodiments, the nucleic acid sequence encoding ICP0 (or a functional variant thereof) is operably linked to a chemically inducible promoter (as described herein); optionally wherein: the nucleic acid sequence encoding UL8 (or a functional variant thereof) is operably linked to a chemically inducible promoter (as described herein); and/or the nucleic acid sequence encoding UL29 (or a functional variant thereof) is operably linked to a chemically inducible promoter (as described herein); and/or the nucleic acid sequence encoding UL30 (or a functional variant thereof) is operably linked to a chemically inducible promoter (as described herein); and/or the nucleic acid sequence encoding UL42 (or a functional variant thereof) is operably linked to a chemically inducible promoter (as described herein); and/or the nucleic acid sequence encoding UL5 (or a functional variant thereof) is operably linked to a chemically inducible promoter (as described herein); and/or the nucleic acid sequence encoding UL52 (or a functional variant thereof) is operably linked to a chemically inducible promoter (as described herein); and/or the nucleic acid sequence encoding UL12 (or a functional variant thereof) is operably linked to a chemically inducible promoter (as described herein); and/or the nucleic acid sequence encoding ICP4 (or a functional variant thereof) is operably linked to a chemically inducible promoter (as described herein); and/or the nucleic acid sequence encoding ICP22 (or a functional variant thereof) is operably linked to a chemically inducible promoter (as described herein).

In some embodiments, the nucleic acid sequence encoding UL12 (or a functional variant thereof) is operably linked to a chemically inducible promoter (as described herein); optionally wherein: the nucleic acid sequence encoding UL8 (or a functional variant thereof) is operably linked to a chemically inducible promoter (as described herein); and/or the nucleic acid sequence encoding UL29 (or a functional variant thereof) is operably linked to a chemically inducible promoter (as described herein); and/or the nucleic acid sequence encoding UL30 (or a functional variant thereof) is operably linked to a chemically inducible promoter (as described herein); and/or the nucleic acid sequence encoding UL42 (or a functional variant thereof) is operably linked to a chemically inducible promoter (as described herein); and/or the nucleic acid sequence encoding UL5 (or a functional variant thereof) is operably linked to a chemically inducible promoter (as described herein); and/or the nucleic acid sequence encoding UL52 (or a functional variant thereof) is operably linked to a chemically inducible promoter (as described herein); and/or the nucleic acid sequence encoding ICP0 (or a functional variant thereof) is operably linked to a chemically inducible promoter (as described herein); and/or the nucleic acid sequence encoding ICP4 (or a functional variant thereof) is operably linked to a chemically inducible promoter (as described herein); and/or the nucleic acid sequence encoding ICP22 (or a functional variant thereof) is operably linked to a chemically inducible promoter (as described herein).

In some embodiments, the nucleic acid sequence encoding ICP4 (or a functional variant thereof) is operably linked to a chemically inducible promoter (as described herein); optionally wherein: the nucleic acid sequence encoding UL8 (or a functional variant thereof) is operably linked to a chemically inducible promoter (as described herein); and/or the nucleic acid sequence encoding UL29 (or a functional variant thereof) is operably linked to a chemically inducible promoter (as described herein); and/or the nucleic acid sequence encoding UL30 (or a functional variant thereof) is operably linked to a chemically inducible promoter (as described herein); and/or the nucleic acid sequence encoding UL42 (or a functional variant thereof) is operably linked to a chemically inducible promoter (as described herein); and/or the nucleic acid sequence encoding UL5 (or a functional variant thereof) is operably linked to a chemically inducible promoter (as described herein); and/or the nucleic acid sequence encoding UL52 (or a functional variant thereof) is operably linked to a chemically inducible promoter (as described herein); and/or the nucleic acid sequence encoding ICP0 (or a functional variant thereof) is operably linked to a chemically inducible promoter (as described herein); and/or the nucleic acid sequence encoding UL12 (or a functional variant thereof) is operably linked to a chemically inducible promoter (as described herein); and/or the nucleic acid sequence encoding ICP22 (or a functional variant thereof) is operably linked to a chemically inducible promoter (as described herein).

In some embodiments, the nucleic acid sequence encoding ICP22 (or a functional variant thereof) is operably linked to a chemically inducible promoter (as described herein); optionally wherein: the nucleic acid sequence encoding UL8 (or a functional variant thereof) is operably linked to a chemically inducible promoter (as described herein); and/or the nucleic acid sequence encoding UL29 (or a functional variant thereof) is operably linked to a chemically inducible promoter (as described herein); and/or the nucleic acid sequence encoding UL30 (or a functional variant thereof) is operably linked to a chemically inducible promoter (as described herein); and/or the nucleic acid sequence encoding UL42 (or a functional variant thereof) is operably linked to a chemically inducible promoter (as described herein); and/or the nucleic acid sequence encoding UL5 (or a functional variant thereof) is operably linked to a chemically inducible promoter (as described herein); and/or the nucleic acid sequence encoding UL52 (or a functional variant thereof) is operably linked to a chemically inducible promoter (as described herein); and/or the nucleic acid sequence encoding ICP0 (or a functional variant thereof) is operably linked to a chemically inducible promoter (as described herein); and/or the nucleic acid sequence encoding UL12 (or a functional variant thereof) is operably linked to a chemically inducible promoter (as described herein); and/or the nucleic acid sequence encoding ICP4 (or a functional variant thereof) is operably linked to a chemically inducible promoter (as described herein).

In some embodiments, a helper system comprises at least two, at least three, at least four, or at least five chemically inducible promoters. In some embodiments, a helper system comprises 2, 3, 4, or 5 chemically inducible promoters.

In some embodiments, wherein a helper system comprises at least two chemically inducible promoters, two or more of the chemically inducible promoters comprise the same nucleic acid sequence. In some embodiments, wherein a helper system comprises at least two chemically inducible promoters, each of the chemically inducible promoters comprise the same nucleic acid sequence. In some embodiments, wherein a helper system comprises at least two chemically inducible promoters, one or more of the chemically inducible promoters comprises a distinct nucleic acid sequence. In some embodiments, wherein a helper system comprises at least two chemically inducible promoters, each of the chemically inducible promoters comprises a distinct nucleic acid sequence.

A helper system described herein may further comprise a polynucleic acid comprising an expression cassette comprising; (i) a nucleic acid sequence of a promoter (constitutive or inducible, as described herein); and (ii) a nucleic acid sequence encoding a transcriptional activator, wherein the transcriptional activator, when expressed in the presence of a small molecule inducer, binds to a chemically inducible promoter of the engineered cell. In embodiments wherein a helper system comprises two or more distinct chemically inducible promoters, the system may comprise nucleic acid sequences of two or more corresponding transcriptional activators.

In some embodiments, a transcriptional activator is Tet-On 3G. In some embodiments, Tet-On 3G comprises the amino acid sequence of SEQ ID NO: 10. In some embodiments, a transcriptional activator is a functional variant of Tet-On 3G. A functional variant of Tet-On 3G comprises at least 80% identity (at least 85%, at least 90%, at least 95%, at least 96%, at least 97%, at least 98%, or at least 99% identity) with SEQ ID NO: 10 and maintains at least 80% of the function (at least 85%, at least 90%, at least 95%, at least 96%, at least 97%, at least 98%, or at least 99% of the function) of Tet-On 3G (that is, its function as a transcriptional activator).

In some embodiments, a transcriptional activator is TetOff-Advanced. In some embodiments, TetOff-Advanced comprises the amino acid sequence of SEQ ID NO: 11. In some embodiments, a transcriptional activator is a functional variant of TetOff-Advanced. A functional variant of TetOff-Advanced comprises at least 80% identity (at least 85%, at least 90%, at least 95%, at least 96%, at least 97%, at least 98%, or at least 99% identity) with SEQ ID NO: 11 and maintains at least 80% of the function (at least 85%, at least 90%, at least 95%, at least 96%, at least 97%, at least 98%, or at least 99% of the function) of TetOff-Advanced (that is, its function as a transcriptional activator).

In some embodiments, a transcriptional activator is VanR-VP16. In some embodiments, VanR-VP16 comprises the amino acid sequence of SEQ ID NO: 12. In some embodiments, a transcriptional activator is a functional variant of VanR-VP16. A functional variant of VanR-VP16 comprises at least 80% identity (at least 85%, at least 90%, at least 95%, at least 96%, at least 97%, at least 98%, or at least 99% identity) with SEQ ID NO: 12 and maintains at least 80% of the function (at least 85%, at least 90%, at least 95%, at least 96%, at least 97%, at least 98%, or at least 99% of the function) of VanR-VP16 (that is, its function as a transcriptional activator).

In some embodiments, a transcriptional activator is TtgR-VP16. In some embodiments, TtgR-VP16 comprises the amino acid sequence of SEQ ID NO: 13. In some embodiments, a transcriptional activator is a functional variant of TtgR-VP16. A functional variant of TtgR-VP16 comprises at least 80% identity (at least 85%, at least 90%, at least 95%, at least 96%, at least 97%, at least 98%, or at least 99% identity) with SEQ ID NO: 13 and maintains at least 80% of the function (at least 85%, at least 90%, at least 95%, at least 96%, at least 97%, at least 98%, or at least 99% of the function) of TtgR-VP16 (that is, its function as a transcriptional activator).

In some embodiments, a transcriptional activator is PhlF-VP16. In some embodiments, PhlF-VP16 comprises the amino acid sequence of SEQ ID NO: 14. In some embodiments, a transcriptional activator is a functional variant of PhlF-VP16. A functional variant of PhlF-VP16 comprises at least 80% identity (at least 85%, at least 90%, at least 95%, at least 96%, at least 97%, at least 98%, or at least 99% identity) with SEQ ID NO: 14 and maintains at least 80% of the function (at least 85%, at least 90%, at least 95%, at least 96%, at least 97%, at least 98%, or at least 99% of the function) of PhlF-VP16 (that is, its function as a transcriptional activator).

In some embodiments, a transcriptional activator is cTA. In some embodiments, cTA comprises the amino acid sequence of SEQ ID NO: 15. In some embodiments, a transcriptional activator is a functional variant of cTA. A functional variant of cTA comprises at least 80% identity (at least 85%, at least 90%, at least 95%, at least 96%, at least 97%, at least 98%, or at least 99% identity) with SEQ ID NO: 15 and maintains at least 80% of the function (at least 85%, at least 90%, at least 95%, at least 96%, at least 97%, at least 98%, or at least 99% of the function) of cTA (that is, its function as a transcriptional activator).

In some embodiments, a transcriptional activator is rcTA. In some embodiments, rcTA comprises the amino acid sequence of SEQ ID NO: 16. In some embodiments, a transcriptional activator is a functional variant of rcTA. A functional variant of rcTA comprises at least 80% identity (at least 85%, at least 90%, at least 95%, at least 96%, at least 97%, at least 98%, or at least 99% identity) with SEQ ID NO: 16 and maintains at least 80% of the function (at least 85%, at least 90%, at least 95%, at least 96%, at least 97%, at least 98%, or at least 99% of the function) of rcTA (that is, its function as a transcriptional activator).

### C. Exemplary HSV-Helper System Architectures

For exemplary purpose, select HSV-helper system architectures are described below.

### 1. First Exemplary Architecture

In some embodiments, an HSV-helper system comprises one or more stably integrated polynucleic acid collectively comprising a nucleic acid sequence encoding for each of: UL5 (or a functional variant thereof); UL8 (or a functional variant thereof); UL29 (or a functional variant thereof); UL30 (or a functional variant thereof); UL42 (or a functional variant thereof); and UL52 (or a functional variant thereof); each of which is operably linked to a chemically inducible promoter (as described herein). In some embodiments, the one or more polynucleotides of an HSV-helper system described herein further encode for (collectively): ICP0 (or a functional variant thereof); UL12 (or a functional variant thereof); ICP4 (or a functional variant thereof); and ICP22 (or a functional variant thereof). In some embodiments, the sequence encoding for ICP0 (or a functional variant thereof); UL12 (or a functional variant thereof); ICP4 (or a functional variant thereof); and/or ICP22 (or a functional variant thereof) is operably linked to a chemically inducible promoter.

In some embodiments, a helper system comprises a first polynucleic acid, wherein the first polynucleic acid comprises the nucleic acid sequence encoding for UL30 (or a functional variant thereof) and the nucleic acid sequence encoding for UL42 (or a functional variant thereof). In some embodiments, the nucleic acid sequence encoding for UL30 (or a functional variant thereof) and the nucleic acid sequence encoding for UL42 (or a functional variant thereof) are each operably linked to a first chemically inducible promoter. In some embodiments, the first chemically inducible promoter comprises the nucleic acid sequence of one or more of SEQ ID NOs: 1-9. In some embodiments, the first polynucleic acid further comprises a nucleic acid sequence encoding for an internal ribosome entry site (IRES). In some embodiments, the nucleic acid sequence encoding for the IRES separates the nucleic acid sequence encoding for UL30 (or a functional variant thereof) and the nucleic acid sequence encoding for UL42 (or a functional variant thereof). In some embodiments, the first polynucleic acid further comprises a selection marker that is operably linked to a promoter (constitutive or inducible, as described herein).

In some embodiments, a helper system comprises a second stably integrated polynucleic acid, wherein the second stably integrated polynucleic acid comprises the nucleic acid sequence encoding for UL5 (or a functional variant thereof), the nucleic acid sequence encoding for UL8 (or a functional variant thereof), the nucleic acid sequence encoding for UL52 (or a functional variant thereof), and the nucleic acid sequence encoding for UL29 (or a functional variant thereof). In some embodiments, the nucleic acid sequence encoding for UL5 (or a functional variant thereof), the nucleic acid sequence encoding for UL8 (or a functional variant thereof), and the nucleic acid sequence encoding for UL52 are each operably linked to a second chemically inducible promoter, and the nucleic acid sequence encoding for UL29 (or a functional variant thereof) is operably linked to a third chemically inducible promoter. In some embodiments, the second chemically inducible promoter comprises the nucleic acid sequence of one or more of SEQ ID NOs: 1-9. In some embodiments, the third chemically inducible promoter the nucleic acid sequence of one or more of SEQ ID NOs: 1-9. In some embodiments, the second polynucleic acid further comprises a nucleic acid sequence encoding for an internal ribosome entry site (IRES). In some embodiments, the nucleic acid sequence encoding for the IRES separates the nucleic acid sequence encoding for UL8 (or a functional variant thereof) and the nucleic acid sequence encoding UL52 (or a functional variant thereof). In some embodiments, the second polynucleic acid further comprises a selection marker that is operably linked to a promoter (constitutive or inducible, as described herein).

In some embodiments, the helper system comprises a third polynucleic acid, wherein the third polynucleic acid comprises a nucleic acid sequence encoding for a transcriptional activator that, when expressed in the presence of a small molecule inducer, binds to a chemically inducible promoter of the engineered cell. In some embodiments, the transcriptional activator comprises the amino acid sequence of any one of SEQ ID NOs: 10-16. In some embodiments, the third polynucleic acid further comprises a selection marker that is operably linked to a promoter (constitutive or inducible, as described herein).

In some embodiments, a helper system comprises a fourth stably integrated polynucleic acid, wherein the fourth stably integrated polynucleic acid comprises, the nucleic acid sequence encoding for ICP0 (or a functional variant thereof), the nucleic acid sequence encoding for UL12 (or a functional variant thereof), the nucleic acid sequence encoding for ICP4 (or a functional variant thereof), and the nucleic acid sequence encoding for ICP22 (or a functional variant thereof). In some embodiments, the fourth polynucleic acid further comprises a selection marker that is operably linked to a promoter (constitutive or inducible, as described herein).

### 2. Second Exemplary Architecture

In some embodiments, an HSV-helper system architecture is as depicted in FIG. 5.

### II. Engineered Cells

In some aspects, the disclosure relates to engineered cells comprising: (i) one or more polynucleic acids of an AAV vector production system described in Part I; (ii) one or more polynucleic acids of an HSV-helper system described in Part II; or (iii) a combination thereof. In the context of an engineered cell, such a polynucleic acid is considered a heterologous polynucleic acid (i.e., a polynucleic acid sequence that is not found in the cell naturally). In some embodiments, an engineered cell comprises each of the polynucleic acids of an AAV production system described in Part I. In some embodiments, an engineered cell comprises each of the polynucleic acids of an HSV-helper system described in Part II. In some embodiments, an engineered cell comprises: (i) each of the polynucleic acids of an AAV production system described in Part I; and (ii) one or more of the polynucleic acids of an HSV-helper system described in Part II. In some embodiments, an engineered cell comprises: (i) one or more of the polynucleic acids of an AAV production system described in Part I; and (ii) each of the polynucleic acids of an HSV-helper system described in Part II. In some embodiments, an engineered cell comprises: (i) each of the polynucleic acids of an AAV production system described in Part I; and (ii) each of the polynucleic acids of an HSV-helper system described in Part II.

In some embodiments, a heterologous polynucleic acid of an engineered cell is transiently present.

In other embodiments, a polynucleic acid of an AAV vector production system or an HSV-helper system is stably integrated into the genome of the engineered cell. In some embodiments, one or more polynucleic acid of an AAV vector production system is stably integrated into the genome of the engineered cell; one or more polynucleic acids of an HSV-helper system is stably integrated into the genome of the engineered cell; or a combination thereof. In some embodiments, each polynucleic acid of an AAV vector production system is stably integrated into the genome of the engineered cell; and one or more polynucleic acids of an HSV-helper system is stably integrated into the genome of the engineered cell. In some embodiments, one or more polynucleic acid of an AAV vector production system is stably integrated into the genome of the engineered cell; and each of the polynucleic acids of an HSV-helper system is stably integrated into the genome of the engineered cell. In some embodiments, each heterologous polynucleic acid is stably integrated into the genome of the engineered cell. In some embodiments, each heterologous polynucleic acid is stably integrated at a different position within the genome of the engineered cell.

In some embodiments, an engineered cell is derived from a HEK293 cell.

In some embodiments, an engineered cell is derived from a HeLa cell.

In some embodiments, an engineered cell is derived from a BHK cell.

In some embodiments, an engineered cell is derived from a Sf9 cell.

### A. Landing Pad

An engineered cell described herein may further comprise a landing pad. As used herein, the term “landing pad” refers to a heterologous polynucleic acid sequence that facilitates the targeted insertion of a “payload” sequence into a specific locus (or multiple loci) of the cell's genome. Accordingly, the landing pad is integrated into the genome of the cell. A fixed integration site is desirable to reduce the variability between experiments that may be caused by positional epigenetic effects or proximal regulatory elements. The ability to control payload copy number is also desirable to modulate expression levels of the payload without changing any genetic components.

In some embodiments, the landing pad is located at a safe harbor site in the genome of the engineered cell. As used herein, the term “safe harbor site” refers to a location in the genome where genes or genetic elements can be introduced without disrupting the expression or regulation of adjacent genes and/or adjacent genomic elements do not disrupt expression or regulation of the introduced genes or genetic elements. Examples of safe harbor sites are known to those having skill in the art and include, but are not limited to, AAVS1, ROSA26, COSMIC, H11, CCR5, and LiPS-A3S. See e.g., Gaidukov et al., Nucleic Acids Res. 2018 May 4; 46(8): 4072-4086; U.S. Pat. No. 8,980,579 B2; U.S. Pat. No. 10,017,786 B2; U.S. Pat. No. 9,932,607 B2; Pub. No.: US 2013/280222 A; Pub. No.: WO 2017/180669 A1—the entireties of which are incorporated herein. In some embodiments, the safe harbor site is a known site. In other embodiments, the safe harbor site is a previously undisclosed site. See “Methods of Identifying High-Expressing Genomic Loci and Uses Thereof” herein. In some embodiments, an engineered cell described herein comprises a landing pad that is integrated at a safe harbor locus selected from the group consisting of AAVS1, ROSA26, COSMIC, H11, CCR5, and LiPS-A3S.

In some embodiments, the engineered cell is derived from a HEK293 cell. In some embodiments, the engineered HEK293 cell comprises a landing pad that is integrated at a safe harbor locus selected from the group consisting of AAVS1, ROSA26, CCR5, and LiPS-A3S.

In some embodiments, the engineered cell is derived from a BHK cell. In some embodiments, the engineered BHK cell comprises a landing pad that is integrated at a safe harbor locus selected from the group consisting of ROSA26, COSMIC, and H11.

Each of the landing pads described herein comprises at least one recombination site. Recombination sites for various integrases have been identified previously. For example, a landing pad may comprise recombination sites corresponding to a Bxb1 integrase, lambda-integrase, Cre recombinase, Flp recombinase, gamma-delta resolvase, Tn3 resolvase, φC31 integrase, or R4 integrase. Exemplary recombination site sequences are known in the art (e.g., attP, attB, attR, attL, Lox, and Frt).

The landing pads described herein may comprise one or more expression cassettes.

### B. Exemplary Engineered Cells

For exemplary purpose, select engineered cells are described below.

### 1. First Exemplary Engineered Cell

In some embodiments, an engineered cell comprises one or more stably integrated polynucleic acids collectively comprising a nucleic acid sequence encoding for each of: Rep52 (or a functional variant thereof) or Rep40 (or a functional variant thereof); Rep78 (or a functional variant thereof) or Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); and AAP (or a functional variant thereof); each of which is operably linked to a chemically inducible promoter (as described herein). In some embodiments, the engineered cell comprises a chemically inducible promoter comprising the nucleic acid sequence of one or more of SEQ ID NOs: 1-9 operably linked to a nucleic acid sequence encoding for at least one of Rep52 (or a functional variant thereof), Rep40 (or a functional variant thereof), Rep78 (or a functional variant thereof), Rep68 (or a functional variant thereof), E2A (or a functional variant thereof), E4Orf6 (or a functional variant thereof), VARNA (or a functional variant thereof), VP1 (or a functional variant thereof), VP2 (or a functional variant thereof), VP3 (or a functional variant thereof), and AAP (or a functional variant thereof). In some embodiments, the engineered cell comprises at least two chemically inducible promoters, wherein two or more of the at least two chemically inducible promoters comprise the same nucleic acid sequence. In some embodiments, each of the at least two chemically inducible promoters comprise the same nucleic acid sequence.

In some embodiments, the engineered cell comprises a first stably integrated polynucleic acid, wherein the first stably integrated polynucleic acid comprises: (i) a nucleic acid sequence encoding for Rep52 (or a functional variant thereof); a nucleic acid sequence encoding for Rep40 (or a functional variant thereof), or both; and (ii) a nucleic acid sequence encoding for Rep78 (or a functional variant thereof), a nucleic acid sequence encoding for Rep68 (or a functional variant thereof), or both. In some embodiments, the nucleic acid sequence encoding for (i) and the nucleic acid sequence encoding for (ii) are each operably linked to a first chemically inducible promoter (as described herein). In some embodiments, the first chemically inducible promoter comprises the nucleic acid sequence of one or more of SEQ ID NOs: 1-9. In some embodiments, the first stably integrated polynucleic acid further comprises a nucleic acid sequence encoding for an internal ribosome entry site (IRES). In some embodiments, the nucleic acid sequence encoding for the IRES separates the nucleic acid sequence encoding for (i) and the nucleic acid sequence encoding for (ii). In some embodiments, the first stably integrated polynucleic acid further comprises a selection marker that is operably linked to a promoter (constitutive or inducible, as described herein).

In some embodiments, the engineered cell comprises a second stably integrated polynucleic acid, wherein the second stably integrated polynucleic acid comprises the nucleic acid sequences encoding for E2A (or a functional variant thereof), E4Orf6 (or a functional variant thereof), and VARNA (or a functional variant thereof). In some embodiments, the nucleic acid sequences encoding for E2A (or a functional variant thereof), E4Orf6 (or a functional variant thereof), and VARNA (or a functional variant thereof) are each operably linked to a second chemically inducible promoter (as described herein). In some embodiments, the second chemically inducible promoter comprises the nucleic acid sequence of one or more of SEQ ID NOs: 1-9. In some embodiments, the second polynucleic acid further comprises a nucleic acid sequence encoding an IRES. In some embodiments, the nucleic acid sequence encoding for the IRES separates the nucleic acid sequence encoding for E2A (or a functional variant thereof) and the nucleic acid sequence encoding for E4Orf6 (or a functional variant thereof). In some embodiments, the second stably integrated polynucleic acid further comprises a selection marker that is operably linked to a promoter (constitutive or inducible, as described herein).

In some embodiments, the engineered cell comprises a third stably integrated polynucleic acid, wherein the third stably integrated polynucleic acid comprises the nucleic acid sequences encoding for VP1 (or a functional variant thereof), VP2 (or a functional variant thereof), VP3 (or a functional variant thereof), and AAP (or a functional variant thereof). In some embodiments, the third stably integrated polynucleic acid further comprises a selection marker that is operably linked to a promoter. In some embodiments, the nucleic acid sequences encoding for VP1 (or a functional variant thereof), VP2 (or a functional variant thereof), VP3 (or a functional variant thereof), and AAP (or a functional variant thereof) are each operably linked to a third chemically inducible promoter (as described herein). In some embodiments, the third chemically inducible promoter comprises the nucleic acid sequence of one or more of SEQ ID NOs: 1-9.

In some embodiments, the engineered cell further comprises a fourth stably integrated polynucleic acid, wherein the fourth stably integrated polynucleic acid comprises a nucleic acid sequence encoding for a transcriptional activator that, when expressed in the presence of a small molecule inducer, binds to a chemically inducible promoter of the engineered cell. In some embodiments, the transcriptional activator comprises the amino acid sequence of any one of SEQ ID NOs: 10-16. In some embodiments, the fourth stably integrated polynucleic acid further comprises a selection marker that is operably linked to a promoter.

In some embodiments, the engineered cell further comprises a stable landing pad.

In some embodiments, the engineered cell is derived from a HEK293 cell, a HeLa cell, a BHK cell, or a Sf9 cell.

### 2. Second Exemplary Engineered Cell

In some embodiments, an engineered cell comprises one or more stably integrated polynucleic acid collectively comprising a nucleic acid sequence encoding for each of: Rep52 (or a functional variant thereof) or Rep40 (or a functional variant thereof); Rep78 (or a functional variant thereof) or Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); and AAP (or a functional variant thereof); at least one of which is operably linked to a chemically inducible promoter; wherein: (i) the nucleic acid sequence encoding for Rep68 (or a functional variant thereof) lacks the Rep52 start codon; (ii) the nucleic acid sequence encoding for Rep78 (or a functional variant thereof) lacks the Rep52 start codon and the Rep68/40 splice site; (iii) the nucleic acid sequence encoding for Rep52 (or a functional variant thereof) lacks the Rep40 splice site; and (iv) the nucleic acid sequence encoding for VP1 (or a functional variant thereof) lacks start codons for VP2, VP3, and AAP.

In some embodiments, the engineered cell comprises a chemically inducible promoter comprising the nucleic acid sequence of one or more of SEQ ID NOs: 1-9, operably linked to a nucleic acid sequence encoding for at least one of Rep52 (or a functional variant thereof), Rep40 (or a functional variant thereof), Rep78 (or a functional variant thereof), Rep68 (or a functional variant thereof), E2A (or a functional variant thereof), E4Orf6 (or a functional variant thereof), VARNA (or a functional variant thereof), SC-VP1 (or a functional variant thereof), VP2 (or a functional variant thereof), VP3 (or a functional variant thereof), and AAP (or a functional variant thereof). In some embodiments, the engineered cell comprises at least two chemically inducible promoters, wherein two or more of the at least two chemically inducible promoters comprise the same nucleic acid sequence. In some embodiments, each of the at least two chemically inducible promoters comprise the same nucleic acid sequence.

In some embodiments, the engineered cell comprises a first stably integrated polynucleic acid, wherein the first stably integrated polynucleic acid comprises a first polynucleic acid comprises: (i) a nucleic acid sequence encoding for Rep52 (or a functional variant thereof); a nucleic acid sequence encoding for Rep40 (or a functional variant thereof), or both; and (ii) a nucleic acid sequence encoding for Rep78 (or a functional variant thereof), a nucleic acid sequence encoding for Rep68 (or a functional variant thereof), or both. In some embodiments, the first stably integrated polynucleic acid comprises the nucleic acid sequence encoding for Rep52 (or a functional variant thereof) and the nucleic acid sequence encoding for Rep78 (or a functional variant thereof) or Rep68 (or a functional variant thereof). In some embodiments, the nucleic acid sequence encoding for Rep52 (or a functional variant thereof) and the nucleic acid sequence encoding for Rep78 (or a functional variant thereof) or Rep68 (or a functional variant thereof) are each operably linked to a first chemically inducible promoter (as described herein). In some embodiments, the first chemically inducible promoter comprises the nucleic acid sequence of one or more of SEQ ID NOs: 1-9. In some embodiments, the first stably integrated polynucleic acid further comprises a nucleic acid sequence encoding for an internal ribosome entry site (IRES) or a 2A peptide. In some embodiments, the nucleic acid sequence encoding for the IRES or the 2A peptide separates the nucleic acid sequence encoding for Rep52 (or a functional variant thereof) and the nucleic acid sequence encoding for Rep78 (or a functional variant thereof) or Rep68 (or a functional variant thereof). In some embodiments, the first stably integrated polynucleic acid further comprises a selection marker that is operably linked to a promoter (constitutive or inducible, as described herein).

In some embodiments, the engineered cell comprises a second stably integrated polynucleic acid, wherein the second stably integrated polynucleic acid comprises the nucleic acid sequence encoding for E2A (or a functional variant thereof), the nucleic acid sequence encoding for E4Orf6 (or a functional variant thereof), and the nucleic acid sequence encoding for VARNA (or a functional variant thereof). In some embodiments, the nucleic acid sequence encoding for E2A (or a functional variant thereof), the nucleic acid sequence encoding for E4Orf6 (or a functional variant thereof), and the nucleic acid sequence encoding for VARNA (or a functional variant thereof) are each operably linked to the second chemically inducible promoter (as described herein). In some embodiments, the second chemically inducible promoter comprises the nucleic acid sequence of one or more of SEQ ID NOs: 1-9. In some embodiments, the second stably integrated polynucleic acid further comprises a nucleic acid sequence encoding for an internal ribosome entry site (IRES) or a 2A peptide. In some embodiments, the nucleic acid sequence encoding for the IRES or the 2A peptide separates the nucleic acid sequence encoding for E2A and the nucleic acid sequence encoding E4Orf6. In some embodiments, the second stably integrated polynucleic acid further comprises a selection marker that is operably linked to a promoter (constitutive or inducible, as described herein).

In some embodiments, the engineered cell comprises a third stably integrated polynucleic acid, wherein the third stably integrated polynucleic acid comprises the nucleic acid sequence encoding for VP1 (or a functional variant thereof), the nucleic acid sequence encoding for VP2 (or a functional variant thereof), the nucleic acid sequence encoding for VP3 (or a functional variant thereof), and the nucleic acid sequence encoding for AAP (or a functional variant thereof). In some embodiments, the nucleic acid sequence encoding for VP1 (or a functional variant thereof) is operably linked to a third chemically inducible promoter (as described herein) and the nucleic acid sequence encoding for VP2 (or a functional variant thereof), the nucleic acid sequence encoding for VP3 (or a functional variant thereof), and the nucleic acid sequence encoding for AAP (or a functional variant thereof) are each operably linked to a fourth chemically inducible promoter (as described herein). In some embodiments, the third chemically inducible promoter comprises the nucleic acid sequence of one or more of SEQ ID NOs: 1-9. In some embodiments, the fourth chemically inducible promoter comprises the nucleic acid sequence of one or more of SEQ ID NOs: 1-9. In some embodiments, the third stably integrated polynucleic acid further comprises a selection marker that is operably linked to a promoter (constitutive or inducible, as described herein).

In some embodiments, the engineered cell comprises a fourth stably integrated polynucleic acid, wherein the fourth polynucleic acid comprises a nucleic acid sequence encoding for a transcriptional activator that, when expressed in the presence of a small molecule inducer, binds to a chemically inducible promoter of the engineered cell. In some embodiments, the transcriptional activator comprises the amino acid sequence of any one of SEQ ID NOs: 10-16. In some embodiments, the fourth polynucleic molecule further comprises a selection marker that is operably linked to a promoter (constitutive or inducible, as described herein).

In some embodiments, the engineered cell further comprises a stable landing pad.

In some embodiments, the engineered cell is derived from a HEK293 cell, a HeLa cell, a BHK cell, or a Sf9 cell.

### 3. Third Exemplary Engineered Cell

In some embodiments, an engineered cell comprises one or more stably integrated polynucleic acid collectively comprising a nucleic acid sequence encoding for each of: UL5 (or a functional variant thereof); UL8 (or a functional variant thereof); UL29 (or a functional variant thereof); UL30 (or a functional variant thereof); UL42 (or a functional variant thereof); and UL52 (or a functional variant thereof); each of which is operably linked to a chemically inducible promoter. In some embodiments, the engineered cell comprises one or more stably integrated polynucleic acid further comprising UL12 (or a functional variant thereof), ICP0 (or a functional variant thereof), ICP4 (or a functional variant thereof), and ICP22 (or a functional variant thereof) each of which is operably linked to a chemically inducible promoter. In some embodiments, the engineered cell comprises a chemically inducible promoter comprising the nucleic acid sequence of one or more of SEQ ID NOs: 1-9 operably linked to a nucleic acid sequence encoding for at least one of UL5 (or a functional variant thereof), UL8 (or a functional variant thereof), UL29 (or a functional variant thereof), UL30 (or a functional variant thereof), UL42 (or a functional variant thereof), UL52 (or a functional variant thereof), UL12 (or a functional variant thereof), ICP0 (or a functional variant thereof), ICP4 (or a functional variant thereof), and ICP22 (or a functional variant thereof). In some embodiments, the engineered cell comprises at least two chemically inducible promoters, wherein two or more of the at least two chemically inducible promoters comprise the same nucleic acid sequence. In some embodiments, each of the at least two chemically inducible promoters comprise the same nucleic acid sequence.

In some embodiments, the engineered cell comprises a first stably integrated polynucleic acid, wherein the first stably integrated polynucleic acid comprises the nucleic acid sequence encoding for UL30 (or a functional variant thereof) and the nucleic acid sequence encoding for UL42 (or a functional variant thereof). In some embodiments, the nucleic acid sequence encoding for UL30 (or a functional variant thereof) and the nucleic acid sequence encoding for UL42 (or a functional variant thereof) are each operably linked to a first chemically inducible promoter. In some embodiments, the first chemically inducible promoter comprises the nucleic acid sequence of one or more of SEQ ID NOs: 1-9. In some embodiments, the first stably integrated polynucleic acid further comprises a nucleic acid sequence encoding for an internal ribosome entry site (IRES). In some embodiments, the nucleic acid sequence encoding for the IRES separates the nucleic acid sequence encoding for UL30 (or a functional variant thereof) and the nucleic acid sequence encoding for UL42 (or a functional variant thereof). In some embodiments, the first stably integrated polynucleic acid further comprises a selection marker that is operably linked to a promoter (constitutive or inducible, as described herein).

In some embodiments, the engineered cell comprises a second stably integrated polynucleic acid, wherein the second stably integrated polynucleic acid comprises the nucleic acid sequence encoding for UL5 (or a functional variant thereof), the nucleic acid sequence encoding for UL8 (or a functional variant thereof), the nucleic acid sequence encoding for UL52 (or a functional variant thereof), and the nucleic acid sequence encoding for UL29 (or a functional variant thereof). In some embodiments, the nucleic acid sequence encoding for UL5 (or a functional variant thereof), the nucleic acid sequence encoding for UL8 (or a functional variant thereof), and the nucleic acid sequence encoding for UL52 are each operably linked to a second chemically inducible promoter, and the nucleic acid sequence encoding for UL29 (or a functional variant thereof) is operably linked to a third chemically inducible promoter. In some embodiments, the second chemically inducible promoter comprises the nucleic acid sequence of one or more of SEQ ID NOs: 1-9. In some embodiments, the third chemically inducible promoter the nucleic acid sequence of one or more of SEQ ID NOs: 1-9. In some embodiments, the second stably integrated polynucleic acid further comprises a nucleic acid sequence encoding for an internal ribosome entry site (IRES). In some embodiments, the nucleic acid sequence encoding for the IRES separates the nucleic acid sequence encoding for UL8 (or a functional variant thereof) and the nucleic acid sequence encoding UL52 (or a functional variant thereof). In some embodiments, the second stably integrated polynucleic acid further comprises a selection marker that is operably linked to a promoter (constitutive or inducible, as described herein).

In some embodiments, the engineered cell comprises a third polynucleic acid, wherein the third polynucleic acid comprises a nucleic acid sequence encoding for a transcriptional activator that, when expressed in the presence of a small molecule inducer, binds to a chemically inducible promoter of the engineered cell. In some embodiments, the transcriptional activator comprises the amino acid sequence of any one of SEQ ID NOs: 10-16. In some embodiments, the third stably integrated polynucleic acid further comprises a selection marker that is operably linked to a promoter (constitutive or inducible, as described herein).

In some embodiments, the engineered cell comprises a fourth polynucleic acid, wherein the fourth polynucleic acid comprises a nucleic acid sequence encoding for UL12 (or a functional variant thereof), ICP0 (or a functional variant thereof), ICP4 (or a functional variant thereof), and ICP22 (or a functional variant thereof). In some embodiments, the nucleic acid sequence encoding for UL12 (or a functional variant thereof), ICP0 (or a functional variant thereof), ICP4 (or a functional variant thereof) are operably linked to a fourth chemically inducible promoter, and ICP22 (or a functional variant thereof) is operably linked to a fifth chemically inducible promoter. In some embodiments, the fourth chemically inducible promoter comprises the nucleic acid sequence of one or more of SEQ ID NOs: 1-9. In some embodiments, the fifth chemically inducible promoter the nucleic acid sequence of one or more of SEQ ID NOs: 1-9. In some embodiments, the fourth stably integrated polynucleic acid further comprises a nucleic acid sequence encoding for an internal ribosome entry site (IRES). In some embodiments, the nucleic acid sequence encoding for the IRES separates the nucleic acid sequence encoding for UL12 (or a functional variant thereof) and the nucleic acid sequence encoding ICP4 (or a functional variant thereof). In some embodiments, the fourth stably integrated polynucleic acid further comprises a selection marker that is operably linked to a promoter (constitutive or inducible, as described herein).

In some embodiments, the engineered cell further comprises a stable landing pad.

In some embodiments, the engineered cell is derived from a HEK293 cell, a HeLa cell, a BHK cell, or a Sf9 cell.

### IV. Kits

In some aspects, the disclosure relates to kits comprising: (i) an AAV vector production system described herein in Part I; (ii) an HSV-helper system as described herein in Part II; or (iii) both.

In some embodiments, a kit comprises: one or more polynucleic acids collectively comprising an AAV vector production system; one or more polynucleic acids collectively comprising an HSV-helper system; or a combination thereof.

In some embodiments, a kit comprises an engineered cell described in Part III.

In some embodiments, a kit comprises a transfer polynucleic acid. The transfer polynucleic acids described herein comprise a central nucleic acid sequence flanked, on the 5′ end and the 3′ end, by a nucleic acid sequence of a lentivirus long tandem repeat (LTR) or the transposase binding sites (such as Sleeping beauty transposase binding sites, PiggyBac binding sites, or Leap-In binding sites) containing inverted and direct repeats (MJIDRs). Exemplary lentivirus LRTs and transposase I/DRs (such as sleeping beauty IR/DRs) are known to those having ordinary skill in the art.

The central nucleic acid of a transfer polynucleic acid may comprise a nucleic acid sequence of a multiple cloning site. Exemplary multiple cloning sites are known to those having ordinary skill in the art. A multiple cloning site can be used for cloning a payload molecule (or gene of interest)—or an expression cassette encoding a payload molecule—into the transfer polynucleic acid prior to the generation of viral vectors in a host cell.

In some embodiments, a kit further comprises a small molecule inducer corresponding to a chemically inducible promoter of the AAV vector production system or a chemically inducible promoter of an HSV-helper system. In some embodiments, a small molecule inducer is tetracycline, doxycycline, cumate, ABA, CRY2-CIB1, DAPG, mifepristone, lactose, or arabinose.

Exemplary kits are provided below.

### A. First Exemplary Kit

In some embodiments, a kit comprises an engineered cell comprising one or more stably integrated polynucleic acids collectively comprising a nucleic acid sequence encoding for each of: Rep52 (or a functional variant thereof) or Rep40 (or a functional variant thereof); Rep78 (or a functional variant thereof) or Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); and AAP (or a functional variant thereof); at least one of which is operably linked to a chemically inducible promoter (as described herein).

In some embodiments, the kit further comprises a transfer polynucleic acid molecule comprising, from 5′ to 3′: (i) a nucleic acid sequence of a 5′ AAV inverted tandem repeat (ITR); (ii) a multiple cloning site; and (iii) a nucleic acid sequence of a 3′ AAV inverted tandem repeat (ITR). In some embodiments, the transfer polynucleic acid is a plasmid or a vector.

In some embodiments, the kit further comprises a small molecule inducer corresponding to a chemically inducible promoter of the engineered cell.

In some embodiments, the engineered cell comprises a chemically inducible promoter comprising the nucleic acid sequence of one or more of SEQ ID NOs: 1-9 operably linked to the nucleic acid sequence encoding for at least one of Rep52 (or a functional variant thereof), Rep40 (or a functional variant thereof), Rep78 (or a functional variant thereof), Rep68 (or a functional variant thereof), E2A (or a functional variant thereof), E4Orf6 (or a functional variant thereof), VARNA (or a functional variant thereof), VP1 (or a functional variant thereof), VP2 (or a functional variant thereof), VP3 (or a functional variant thereof), and AAP (or a functional variant thereof).

In some embodiments, the engineered cell comprises a chemically inducible promoter comprising the nucleic acid sequence of one or more of SEQ ID NOs: 1-3 operably linked to the nucleic acid sequence encoding for at least one of Rep52 (or a functional variant thereof), Rep40 (or a functional variant thereof), Rep78 (or a functional variant thereof), Rep68 (or a functional variant thereof), E2A (or a functional variant thereof), E4Orf6 (or a functional variant thereof), VARNA (or a functional variant thereof), VP1 (or a functional variant thereof), VP2 (or a functional variant thereof), VP3 (or a functional variant thereof), and AAP (or a functional variant thereof).

In some embodiments, the nucleic acid sequence encoding for Rep52 (or a functional variant thereof) or Rep40 (or a functional variant thereof), the nucleic acid sequence encoding for Rep78 (or a functional variant thereof) or Rep68 (or a functional variant thereof), the nucleic acid sequence encoding for E2A (or a functional variant thereof), the nucleic acid sequence encoding for EOrf6 (or a functional variant thereof), the nucleic acid sequence encoding for VARNA (or a functional variant thereof), the nucleic acid sequence encoding for VP1 (or a functional variant thereof), the nucleic acid sequence encoding for VP2 (or a functional variant thereof), the nucleic acid sequence encoding for VP3 (or a functional variant thereof), and the nucleic acid sequence encoding for AAP (or a functional variant thereof) are each operably linked to a chemically inducible promoter comprising the nucleic acid sequence of one or more of SEQ ID NOs: 1-9. In some embodiments, the engineered cell comprises at least two chemically inducible promoters. In some embodiments, two or more of the at least two chemically inducible promoters are distinct.

In some embodiments, the nucleic acid sequence encoding for Rep52 (or a functional variant thereof) or Rep40 (or a functional variant thereof), the nucleic acid sequence encoding for Rep78 (or a functional variant thereof) or Rep68 (or a functional variant thereof), the nucleic acid sequence encoding for E2A (or a functional variant thereof), the nucleic acid sequence encoding for EOrf6 (or a functional variant thereof), the nucleic acid sequence encoding for VARNA (or a functional variant thereof), the nucleic acid sequence encoding for VP1 (or a functional variant thereof), the nucleic acid sequence encoding for VP2 (or a functional variant thereof), the nucleic acid sequence encoding for VP3 (or a functional variant thereof), and the nucleic acid sequence encoding for AAP (or a functional variant thereof) are each operably linked to a chemically inducible promoter comprising the nucleic acid sequence of one or more of SEQ ID NOs: 1-3. In some embodiments, the engineered cell comprises at least two chemically inducible promoters. In some embodiments, two or more of the at least two chemically inducible promoters are distinct.

In some embodiments, the kit comprise a polynucleic acid comprising a nucleic acid sequence of a transcriptional activator operably linked to a nucleic acid sequence of a promoter (constitutive or inducible, as described herein), wherein the transcriptional activator, when expressed in the presence of the small molecule inducer, binds to a chemically inducible promoter of the engineered cell, optionally wherein the engineered cell comprises the polynucleic acid comprising the nucleic acid sequence of the transcriptional activator. In some embodiments, the transcriptional activator comprises the amino acid sequence of any one of SEQ ID NOs: 10-16.

In some embodiments, the kit comprise a polynucleic acid comprising a nucleic acid sequence of a transcriptional activator operably linked to a nucleic acid sequence of a promoter (constitutive or inducible, as described herein), wherein the transcriptional activator, when expressed in the presence of the small molecule inducer, binds to a chemically inducible promoter of the engineered cell, optionally wherein the engineered cell comprises the polynucleic acid comprising the nucleic acid sequence of the transcriptional activator. In some embodiments, the transcriptional activator comprises the amino acid sequence of any one of SEQ ID NOs: 10-12.

In some embodiments, the kit comprises a small molecule inducer corresponding to a chemically inducible promoter of the lentiviral vector production. In some embodiments, a small molecule inducer is tetracycline, doxycycline, cumate, ABA, CRY2-CIB1, DAPG, mifepristone, lactose, or arabinose. In some embodiments, a kit comprises tetracycline or doxycycline.

### B. Second Exemplary Kit

In some embodiments, a kit comprises an engineered cell comprising one or more stably integrated polynucleic acid collectively comprising a nucleic acid sequence encoding for each of: UL5 (or a functional variant thereof); UL8 (or a functional variant thereof); UL29 (or a functional variant thereof); UL30 (or a functional variant thereof); UL42 (or a functional variant thereof); and UL52 (or a functional variant thereof); at least one of which is operably linked to a chemically inducible promoter (as described herein). In some embodiments, the one or more stably integrated polynucleic acid(s) further encode for (collectively): ICP0 (or a functional variant thereof); UL12 (or a functional variant thereof); ICP4 (or a functional variant thereof); and ICP22 (or a functional variant thereof). In some embodiments, the sequence encoding for ICP0 (or a functional variant thereof); UL12 (or a functional variant thereof); ICP4 (or a functional variant thereof); and/or ICP22 (or a functional variant thereof) is operably linked to a chemically inducible promoter.

In some embodiments, a kit further comprises a transfer polynucleic acid molecule comprising, from 5′ to 3′: (i) a nucleic acid sequence of a 5′ AAV inverted tandem repeat (ITR); (ii) a multiple cloning site; and (iii) a nucleic acid sequence of a 3′ AAV inverted tandem repeat (ITR). In some embodiments, the transfer polynucleic acid is a plasmid or a vector.

In some embodiments, the kit further comprises a small molecule inducer corresponding to a chemically inducible promoter of the engineered cell.

In some embodiments, the engineered cell comprises a chemically inducible promoter comprising the nucleic acid sequence of one or more of SEQ ID NOs: 1-9 operably linked to the nucleic acid sequence encoding for at least one of UL5 (or a functional variant thereof), UL8 (or a functional variant thereof), UL29 (or a functional variant thereof), UL30 (or a functional variant thereof), UL42 (or a functional variant thereof), and UL52 (or a functional variant thereof). In some embodiments, the nucleic acid sequence encoding for UL5 (or a functional variant thereof), the nucleic acid sequence encoding for UL8 (or a functional variant thereof), the nucleic acid sequence encoding for UL29 (or a functional variant thereof), the nucleic acid sequence encoding for UL30 (or a functional variant thereof), the nucleic acid sequence encoding for UL42 (or a functional variant thereof), the nucleic acid sequence encoding for UL52 (or a functional variant thereof), the nucleic acid sequence encoding for ICP0 (or a functional variant thereof), the nucleic acid sequence encoding for UL12 (or a functional variant thereof), the nucleic acid sequence encoding for ICP4 (or a functional variant thereof), and the nucleic acid sequence encoding for ICP22 (or a functional variant thereof) are each operably linked to a chemically inducible promoter comprising the nucleic acid sequence of one or more of SEQ ID NOs: 1-9. In some embodiments, the engineered cell comprises at least two chemically inducible promoters. In some embodiments, two or more of the at least two chemically inducible promoters are distinct.

In some embodiments, the kit comprise a polynucleic acid comprising a nucleic acid sequence of a transcriptional activator operably linked to a nucleic acid sequence of a promoter (constitutive or inducible, as described herein), wherein the transcriptional activator, when expressed in the presence of the small molecule inducer, binds to a chemically inducible promoter of the engineered cell, optionally wherein the engineered cell comprises the polynucleic acid comprising the nucleic acid sequence of the transcriptional activator. In some embodiments, the transcriptional activator comprises the amino acid sequence of any one of SEQ ID NOs: 10-16. In some embodiments, the kit comprises the small molecule inducer doxycycline or tetracycline.

### V. Methods

In some aspects, the disclosure relates to methods of producing an AAV vector in an engineered cell (e.g., an engineered cell, as described herein). In some embodiments, the method comprises expressing: Rep52 (or a functional variant thereof) or Rep40 (or a functional variant thereof); Rep78 (or a functional variant thereof) or Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); and AAP (or a functional variant thereof) in the engineered cell. In some embodiments, the method comprises expressing UL5 (or a functional variant thereof), UL8 (or a functional variant thereof), UL29 (or a functional variant thereof), UL30 (or a functional variant thereof), UL42 (or a functional variant thereof), and UL52 (or a functional variant thereof) in the engineered cell. In some embodiments, the method comprises expressing ICP0 (or a functional variant thereof), UL12 (or a functional variant thereof), ICP4 (or a functional variant thereof), and ICP22 (or a functional variant thereof) in the engineered cell.

In some embodiments, the engineered cell comprises: one or more polynucleic acids of an inducible AAV vector production system (as described herein); one or more polynucleic acids of an inducible HSV-helper system (as described herein); or both. In some embodiments, the engineered cell comprises an inducible AAV vector production system (as described herein). In some embodiments, the engineered cell comprises an inducible HSV-helper system as described herein. In some embodiments, the engineered cell comprises an inducible AAV vector production system (as described herein) and an inducible HSV-helper system as described herein.

Exemplary kits are provided below.

### A. First Exemplary Method

In some embodiments, a method of producing an AAV vector comprises: (a) introducing a transfer polynucleic acid into the engineered cell comprising one or more stably integrated polynucleic acids collectively comprising a nucleic acid sequence encoding for each of: Rep52 (or a functional variant thereof) or Rep40 (or a functional variant thereof); Rep78 (or a functional variant thereof) or Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); and AAP (or a functional variant thereof); at least one of which is operably linked to a chemically inducible promoter (as described herein); and (b) contacting the engineered cell with a small molecule inducer corresponding to a chemically inducible promoter of the engineered cell, thereby inducing expression of: Rep52 (or a functional variant thereof) or Rep40 (or a functional variant thereof); Rep78 (or a functional variant thereof) or SC-Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); and AAP (or a functional variant thereof); wherein the engineered cell comprises a heterologous polynucleic acid comprising a nucleic acid sequence of a transcriptional activator operably linked to a nucleic acid sequence of a promoter (constitutive or inducible, as described herein), wherein the transcriptional activator, when expressed in the presence of the small molecule inducer, binds to a chemically inducible promoter of the engineered cell; wherein (b) occurs before, concurrently with, or after (a).

In some embodiments, the engineered cell comprises a chemically inducible promoter comprising the nucleic acid sequence of one or more of SEQ ID NOs: 1-9 operably linked to the nucleic acid sequence encoding for at least one of Rep52 (or a functional variant thereof), Rep40 (or a functional variant thereof), Rep78 (or a functional variant thereof), Rep68 (or a functional variant thereof), E2A (or a functional variant thereof), E4Orf6 (or a functional variant thereof), VARNA (or a functional variant thereof), VP1 (or a functional variant thereof), VP2 (or a functional variant thereof), VP3 (or a functional variant thereof), and AAP (or a functional variant thereof). In some embodiments, the nucleic acid sequence encoding for Rep52 (or a functional variant thereof) or Rep40 (or a functional variant thereof), the nucleic acid sequence encoding for Rep78 (or a functional variant thereof) or Rep68 (or a functional variant thereof), the nucleic acid sequence encoding for E2A (or a functional variant thereof), the nucleic acid sequence encoding for EOrf6 (or a functional variant thereof), the nucleic acid sequence encoding for VARNA (or a functional variant thereof), the nucleic acid sequence encoding for VP1 (or a functional variant thereof), the nucleic acid sequence encoding for VP2 (or a functional variant thereof), the nucleic acid sequence encoding for VP3 (or a functional variant thereof), and the nucleic acid sequence encoding for AAP (or a functional variant thereof) are each operably linked to a chemically inducible promoter comprising the nucleic acid sequence of one or more of SEQ ID NOs: 1-9. In some embodiments, the engineered cell comprises at least two chemically inducible promoters. In some embodiments, two or more of the at least two chemically inducible promoters are distinct.

In some embodiments, the transcriptional activator comprises the amino acid sequence of any one of SEQ ID NOs: 10-16. In some embodiments, the small molecule inducer is doxycycline, tetracycline, DAPG, cumate lactose, or arabinose.

In some embodiments, the engineered cell comprises a chemically inducible promoter comprising the nucleic acid sequence of one or more of SEQ ID NOs: 1-3 operably linked to the nucleic acid sequence encoding for at least one of Rep52 (or a functional variant thereof), Rep40 (or a functional variant thereof), Rep78 (or a functional variant thereof), Rep68 (or a functional variant thereof), E2A (or a functional variant thereof), E4Orf6 (or a functional variant thereof), VARNA (or a functional variant thereof), VP1 (or a functional variant thereof), VP2 (or a functional variant thereof), VP3 (or a functional variant thereof), and AAP (or a functional variant thereof). In some embodiments, the nucleic acid sequence encoding for Rep52 (or a functional variant thereof) or Rep40 (or a functional variant thereof), the nucleic acid sequence encoding for Rep78 (or a functional variant thereof) or Rep68 (or a functional variant thereof), the nucleic acid sequence encoding for E2A (or a functional variant thereof), the nucleic acid sequence encoding for EOrf6 (or a functional variant thereof), the nucleic acid sequence encoding for VARNA (or a functional variant thereof), the nucleic acid sequence encoding for VP1 (or a functional variant thereof), the nucleic acid sequence encoding for VP2 (or a functional variant thereof), the nucleic acid sequence encoding for VP3 (or a functional variant thereof), and the nucleic acid sequence encoding for AAP (or a functional variant thereof) are each operably linked to a chemically inducible promoter comprising the nucleic acid sequence of one or more of SEQ ID NOs: 1-3. In some embodiments, the engineered cell comprises at least two chemically inducible promoters. In some embodiments, two or more of the at least two chemically inducible promoters are distinct.

In some embodiments, the transcriptional activator comprises the amino acid sequence of any one of SEQ ID NOs: 10-12. In some embodiments, the small molecule inducer is doxycycline or tetracycline.

### B. Second Exemplary Method

In some embodiments, a method of producing an AAV vector comprises: (a) introducing a transfer polynucleic acid into the engineered cell comprising one or more stably integrated polynucleic acid collectively comprising a nucleic acid sequence encoding for each of: UL5 (or a functional variant thereof); UL8 (or a functional variant thereof); UL29 (or a functional variant thereof); UL30 (or a functional variant thereof); UL42 (or a functional variant thereof); and UL52 (or a functional variant thereof); at least one of which is operably linked to a chemically inducible promoter; and (b) contacting the engineered cell with a small molecule inducer corresponding to a chemically inducible promoter of the engineered cell, thereby inducing expression of: UL5 (or a functional variant thereof), UL8 (or a functional variant thereof), UL29 (or a functional variant thereof), UL30 (or a functional variant thereof), UL42 (or a functional variant thereof), and UL52 (or a functional variant thereof); wherein the engineered cell comprises a heterologous polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the engineered cell; wherein (b) occurs before, concurrently with, or after (a). In some embodiments, the one or more stably integrated polynucleic acid(s) of the engineered cell further comprise a nucleic acid sequence encoding for ICP0 (or a functional variant thereof), UL12 (or a functional variant thereof), ICP4 (or a functional variant thereof), and/or ICP22 (or a functional variant thereof).

In some embodiments, the engineered cell comprises the engineered cell comprises one or more polynucleic acids collectively comprising a nucleic acid sequence encoding for each of: Rep52 (or a functional variant thereof) or Rep40 (or a functional variant thereof); Rep78 (or a functional variant thereof) or Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); and AAP (or a functional variant thereof).

In some embodiments, the engineered cell comprises one or more stably integrated polynucleic acids collectively comprising a nucleic acid sequence encoding for each of: Rep52 (or a functional variant thereof) or Rep40 (or a functional variant thereof); Rep78 (or a functional variant thereof) or Rep68 (or a functional variant thereof); E2A (or a functional variant thereof); E4Orf6; VARNA (or a functional variant thereof); VP1 (or a functional variant thereof); VP2 (or a functional variant thereof); VP3 (or a functional variant thereof); and AAP (or a functional variant thereof).

In some embodiments, the method further comprises: (c) introducing one or more polynucleic acids collectively comprising a nucleic acid sequence encoding for each of: Rep52 (or a functional variant thereof) or Rep40 (or a functional variant thereof); Rep78 (or a functional variant thereof) or Rep68 (or a functional variant thereof); E2A (or a functional variant thereof); E4Orf6 (or a functional variant thereof); VARNA (or a functional variant thereof); VP1 (or a functional variant thereof); VP2 (or a functional variant thereof); VP3 (or a functional variant thereof); and AAP (or a functional variant thereof); wherein (c) occurs prior to, concurrently with, or after (a) or (b).

In some embodiments, the engineered cell comprises a chemically inducible promoter comprising the nucleic acid sequence of one or more of SEQ ID NOs: 1-9 operably linked to the nucleic acid sequence encoding for at least one of UL5 (or a functional variant thereof), UL8 (or a functional variant thereof), UL29 (or a functional variant thereof), UL30 (or a functional variant thereof), UL42 (or a functional variant thereof), UL52 (or a functional variant thereof), ICP0 (or a functional variant thereof), UL12 (or a functional variant thereof), ICP4 (or a functional variant thereof), and ICP22 (or a functional variant thereof). In some embodiments, the nucleic acid sequence encoding for Rep52 (or a functional variant thereof) or Rep40 (or a functional variant thereof), the nucleic acid sequence encoding for UL5 (or a functional variant thereof), the nucleic acid sequence encoding for UL8 (or a functional variant thereof), the nucleic acid sequence encoding for UL29 (or a functional variant thereof), the nucleic acid sequence encoding for UL30 (or a functional variant thereof), the nucleic acid sequence encoding for UL42 (or a functional variant thereof), the nucleic acid sequence encoding for UL52 (or a functional variant thereof), the nucleic acid sequence encoding for ICP0 (or a functional variant thereof), the nucleic acid sequence encoding for UL12 (or a functional variant thereof), the nucleic acid sequence encoding for ICP4 (or a functional variant thereof), and the nucleic acid sequence encoding for ICP22 (or a functional variant thereof) are each operably linked to a chemically inducible promoter comprising the nucleic acid sequence of one or more of SEQ ID NOs: 1-9. In some embodiments, the engineered cell comprises at least two chemically inducible promoters. In some embodiments, two or more of the at least two chemically inducible promoters are distinct.

In some embodiments, the transcriptional activator comprises the amino acid sequence of any one of SEQ ID NOs: 10-16. In some embodiments, the small molecule inducer is doxycycline, tetracycline, cumate, lactose, or arabinose.

In some embodiments, the engineered cell comprises a chemically inducible promoter comprising the nucleic acid sequence of one or more of SEQ ID NOs: 1-3 operably linked to the nucleic acid sequence encoding for at least one of UL5 (or a functional variant thereof), UL8 (or a functional variant thereof), UL29 (or a functional variant thereof), UL30 (or a functional variant thereof), UL42 (or a functional variant thereof), UL52 (or a functional variant thereof), ICP0 (or a functional variant thereof), UL12 (or a functional variant thereof), ICP4 (or a functional variant thereof), and ICP22 (or a functional variant thereof). In some embodiments, the nucleic acid sequence encoding for Rep52 (or a functional variant thereof) or Rep40 (or a functional variant thereof), the nucleic acid sequence encoding for UL5 (or a functional variant thereof), the nucleic acid sequence encoding for UL8 (or a functional variant thereof), the nucleic acid sequence encoding for UL29 (or a functional variant thereof), the nucleic acid sequence encoding for UL30 (or a functional variant thereof), the nucleic acid sequence encoding for UL42 (or a functional variant thereof), the nucleic acid sequence encoding for UL52 (or a functional variant thereof), the nucleic acid sequence encoding for ICP0 (or a functional variant thereof), the nucleic acid sequence encoding for UL12 (or a functional variant thereof), the nucleic acid sequence encoding for ICP4 (or a functional variant thereof), and the nucleic acid sequence encoding for ICP22 (or a functional variant thereof) are each operably linked to a chemically inducible promoter comprising the nucleic acid sequence of one or more of SEQ ID NOs: 1-3. In some embodiments, the engineered cell comprises at least two chemically inducible promoters. In some embodiments, two or more of the at least two chemically inducible promoters are distinct.

In some embodiments, the transcriptional activator comprises the amino acid sequence of any one of SEQ ID NOs: 10-12. In some embodiments, the small molecule inducer is doxycycline or tetracycline.

### EXAMPLES

### Example 1. Inducible Control Over AAV Vector Production

This approach uses a small-molecule inducer to control transcription of genes required for AAV production, including cytostatic or cytotoxic Rep, E2A, E4orf6. The following design is based on using doxycycline (FIGS. 1-2, TABLEs 1-3). In the absence of doxycycline there will be minimal if any expression of the genes necessary for AAV production, and this will minimize cell stress and toxicity associated with AAV production. This results in the ability to generate stable AAV producer cell lines.

Plasmids were designed to remove or minimize native promoter elements to eliminate uncontrolled expression from Rep, Cap, E2A, and E4orf6. Further modifications include linking the Rep52/40 to Rep78/68 and E2A to E4orf6 using an attenuated IRES sequence to minimize unwanted basal expression from multiple transcription units and provide the correct stoichiometry of Rep52/40:Rep78/68 and E2A:E4orf6 respectively.

Adherent HEK293FT cells were co-transfected with EGFP-expressing transfer plasmid, pRepCap, and pHelper. Inducible variants of pRep, pCap and pHelper replaced the ‘wild type’ plasmids to test the ability to produce AAV through dox induction. Doxycycline was added at the time of transfection. Control samples containing only ‘wild type’ AAV2 pRepCap and pHelper plasmids or a negative control transfection mix without DNA were also prepared. 72 hours after transfection, AAV was harvested by three freeze thaw cycles in a dry ice isopropanol bath. Virus stock was serially diluted 1-, 10- and 100-fold and 10 μL of resulting viral stocks was transduced by addition to 5e4 HEK293FT cells plated in a 96-well plate. 72 hours after transduction, transduced cells were harvested and percentage of EGFP positive cells was determined by flow cytometry and used to calculate transducing units per mL (TU/mL).

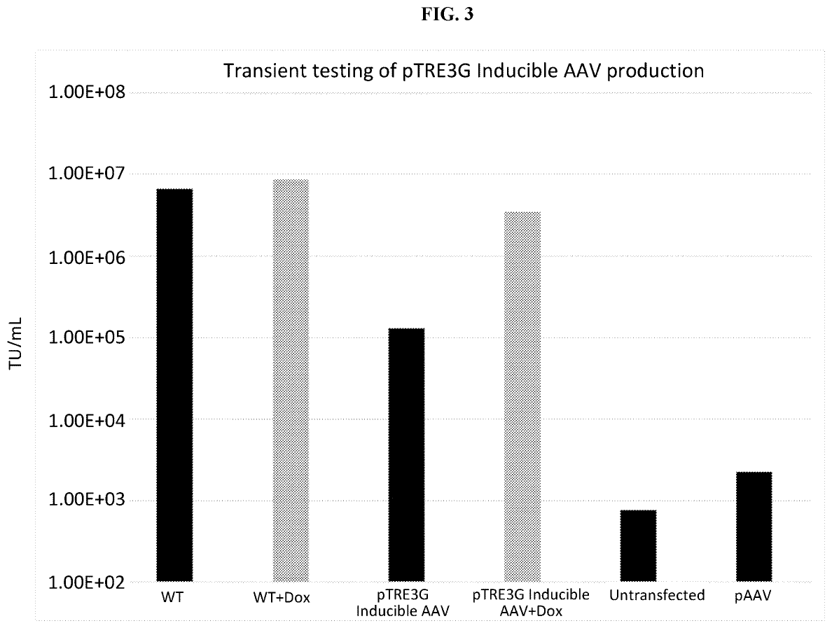

Results are shown in FIG. 3. The presence of AAV particles in the uninduced samples may be due to leakiness of the TRE3G system in transient format. The inducible pRep, pHelper, and pCap genes can be integrated into various HEK293 and HeLa cell lines and the resulting packaging cell lines can be characterized for their inducibility and AAV productivity.

### Example 2. Refactored AAV

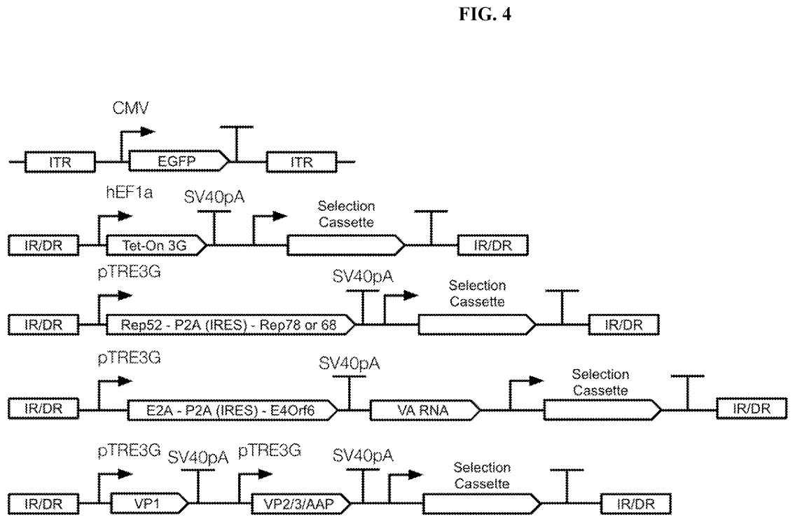

Current systems of AAV production rely on DNA sequences that include all regulatory elements and splice variants of Rep, Cap, and Helper genes. Minimizing this system to the essential genes for AAV production provides the possibility of more control and higher probability of establishing a stable producer cell line. The essential genes for AAV production are the large Rep78, the small Rep52, E2A, E4orf6, and all of the Cap genes. It was hypothesized that expressing only the essential Rep, Cap, and helper genes in a context outside of their native architecture would grant better control over gene expression (FIG. 4, TABLEs 1-3). This enhanced control would more tightly regulate expression of cytotoxic Rep and helper proteins improving cell health and leading to a higher probability of generating a stable packaging cell line.

### Example 3. HSV-Helper System Inducible AAV

Herpesviruses can be used as helper viruses for the production of adeno-associated virus (AAV). HSV replication genes UL5, UL8, UL9, UL29, UL30, UL42, and UL52 are the only required HSV factors for productive AAV replication. Currently infection is used in HSV based AAV production, integration of these genes will remove the need for virus. It was hypothesized that inducible expression of these genes will provide the necessary proteins for production of AAV in a stable cell line providing an alternative platform for AAV manufacturing (FIG. 5, TABLEs 1-3). Benefits of this platform may include higher packaging efficiency and higher yield as compared to adenovirus-based systems.

Adherent HEK293T cells were co-transfected with EGFP-expressing transfer plasmid, pRepCap, and pHelper. Inducible variants of pRep, pCap, pHelper replaced the ‘wild type’ plasmids to determine the ability to produce AAV through dox induction. pHelper inducible variants were replaced with HSV Doxycycline was added at the time of transfection. Control samples containing only ‘wild type’ AAV2 pRepCap and pHelper plasmids or a negative control transfection mix without DNA were also prepared. 72 hours after transfection, AAV was harvested by three freeze thaw cycles in a dry ice isopropanol bath. Virus stocks were treated with DNAse and proteinase to release genomes from packaged AAV. The lysed samples were serially diluted to 1.0e5 and 1e6 fold and genome copy number was determined by ddPCR using sequence specific probes to the GFP gene within the packaged AAV particles and results were calculated as viral genomes/mL (vg/mL).

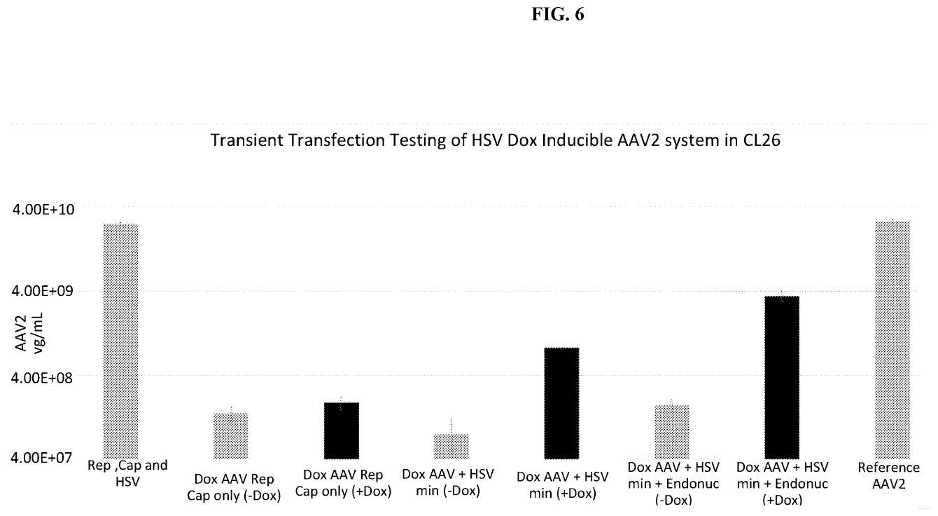

Results are shown in FIG. 6. Inclusion of HSV-based helper system led to AAV production that is inducible, indicated by the increase level of AAV in the presence of doxycycline. The presence of AAV particles in the uninduced samples may be due to leakiness of the TRE3G system in transient format. The inducible pRep, HSV, and pCap genes will be integrated into various HEK293 and HeLa cell lines and the resulting packaging cell lines will be characterized for their inducibility and AAV productivity.

TABLE 1
Exemplary chemically inducible promoters/operators.

| SEQ | | |
|---|---|---|
| ID | | |
| NO: | Name | Sequence |
| 1 | pTREtight | CTCGAGTTTACTCCCTATCAGTGATAGAGAACGTATGTCGAGTTTACTCCCT |
| | | ATCAGTGATAGAGAACGATGTCGAGTTTACTCCCTATCAGTGATAGAGAAC |
| | | GTATGTCGAGTTTACTCCCTATCAGTGATAGAGAACGTATGTCGAGTTTACT |
| | | CCCTATCAGTGATAGAGAACGTATGTCGAGTTTATCCCTATCAGTGATAGAG |
| | | AACGTATGTCGAGTTTACTCCCTATCAGTGATAGAGAACGTATGTCGAGGTA |
| | | GGCGTGTACGGTGGGAGGCCTATATAAGCAGAGCTCGTTTAGTGAACCGTC |
| | | AGATCGCCTGGAGAATTCGAGCTCGGTACCCGGGGA |
| 2 | pTRE3G | GTTTACTCCCTATCAGTGATAGAGAACGTATGAAGAGTTTACTCCCTATCAG |
| | | TGATAGAGAACGTATGCAGACTTTACTCCCTATCAGTGATAGAGAACGTAT |
| | | AAGGAGTTTACTCCCTATCAGTGATAGAGAACGTATGACCAGTTTACTCCCT |
| | | ATCAGTGATAGAGAACGTATCTACAGTTTACTCCCTATCAGTGATAGAGAAC |
| | | GTATATCCAGTTTACTCCCTATCAGTGATAGAGAACGTATAAGCTTTAGGCG |
| | | TGTACGGTGGGCGCCTATAAAAGCAGAGCTCGTTTAGTGAACCGTCAGATC |
| | | GCCTGGAGCAATTCCACAACACTTTTGTCTTATACCAACTTTCCGTACCACTT |
| | | CCTACCCTCGTAAAGTCGACACCGGGGCCCAGATCTATCGATCGGCCGGAT |
| | | AACGCCACC |
| 3 | bi-TRE3G | GAATTCTCCAGGCGATCTGACGGTTCACTAAACGAGCTCTGCTTATATAGGC |
| | | CTCCCACCGTACACGCCACCTCGACATACTCGAGTTTACTCCCTATCAGTGA |
| | | TAGAGAACGTATGAAGAGTTTACTCCCTATCAGTGATAGAGAACGTATGCA |
| | | GACTTTACTCCCTATCAGTGATAGAGAACGTATAAGGAGTTTACTCCCTATC |
| | | AGTGATAGAGAACGTATGACCAGTTTACTCCCTATCAGTGATAGAGAACGT |
| | | ATCTACAGTTTACTCCCTATCAGTGATAGAGAACGTATATCCAGTTTACTCC |
| | | CTATCAGTGATAGAGAACGTATAAGCTTTAGGCGTGTACGGTGGGCGCCTA |
| | | TAAAAGCAGAGCTCGTTTAGTGAACCGTCAGATCGCCTGGAGCAATTCCAC |
| | | AACACTTTTGTCTTATACCAACTTTCCGTACCACTTCCTACCCTCGTAAAGTC |
| | | GACACCGGGGCCCAGATCTCCGCGGGGATCC |
| 4 | VanR | ATTGGATCCAAT |
| | operator | |
| 5 | TtgR | TATTTACAAACAACCATGAATGTAAGTA |
| | operator | |
| 6 | Gal4 UAS | CGGAGTACTGTCCTCCGA |
| | (for CID | |
| | systems) | |
| 7 | PhIF | ATGATACGAAACGTACCGTATCGTTAAGGT |
| | operator | |
| 8 | CymR | AGAAACAAACCAACCTGTCTGTATTA |
| | operator | |
| | v1 | |
| 9 | CymR | AACAAACAGACAATCTGGTCTGTTTGTA |
| | operator | |
| | v2 | |

TABLE 2
Exemplary transcriptional activators.

| SEQ | | |
|---|---|---|
| ID | | |
| NO: | Name | Sequence |
| 10 | Tet-On | MSRLDKSKVINSALELLNGVGIEGLTTRKLAQKLGVEQPTLYWHVKNKRALLD |
| | 3G | ALPIEMLDRHHTHSCPLEGESWQDFLRNNAKSYRCALLSHRDGAKVHLGTRPT |
| | | EKQYELTLENQLAFLCQQGFSLENALYALSAVGHFTLGCVLEEQEHQVAKEER |
| | | ETPTTDSMPPLLKQAIELFDRQGAEPAFLFGLELIICGLEKQLKCESGGPTDALD |
| | | DFDLDMLPADALDDFDLDMLPADALDDFDLDMLPG |
| 11 | TetOff- | MSRLDKSKVINSALELLNEVGIEGLTTRKLAQKLGVEQPTLYWHVKNKRALLD |
| | Advanced | ALAIEMLDRHHTHFCPLEGESWQDFLRNNAKSFRCALLSHRDGAKVHLGTRPT |
| | | EKQYETLENQLAFLCQQGFSLENALYALSAVGHFTLGCVLEDQEHQVAKEERE |
| | | TPTTDSMPPLLRQAIELFDHQGAEPAFLFGLELIICGLEKQLKCESGGPADALDD |
| | | FDLDMLPADALDDFDLDMLPADALDDFDLDMLPG |
| 12 | VanR- | MDMPRIKPGQRVMMALRKMIASGEIKSGERIAEIPTAAALGVSRMPVRIALRSL |
| | VP16 | EQEGLVVRLGARGYAARGVSSDQIRDAIEVRGVLEGFAARRLAERGMTAETHA |
| | | RFVVLIAEGEALFAAGRLNGEDLDRYAAYNQAFHDTLVSAAGNGAVESALAR |
| | | NGFEPFAAAGALALDLMDLSAEYEHLLAAHRQHQAVLDAVSCGDAEGAERIM |
| | | RDHALAAIRNAKVFEAAASAGAPLGAAWSIRADSGGGGPTDALDDFDLDMLP |
| | | ADALDDFDLDMLPADALDDFDLDMLPGPPKKKRKV |
| 13 | TtgR- | MVRRTKEEAQETRAQIIEAAERAFYKRGVARTTLADIAELAGVTRGAIYWHEN |
| | VP16 | NKAELVQALLDSLHETHDHLARASESEDEVDPLGCMRKLLLQVFNELVLDART |
| | | RRINEILHHKCEFTDDMCEIRQQHQSAVLDCHKGITLTLANVVRRGQLPGELDA |
| | | ERAAVAMFAYVDGLIRRWLLLPDSVDLLGDVEKWVDTGLDMLRLSPALRKSG |
| | | GGGPTDALDDFDLDMLPADALDDFDLDMLPADALDDFDLDMLPGPPKKKRKV |
| 14 | PhlF- | MARTPSRSSIGSLRSPHTHKAILTSTIEILKECGYSGLSIESVARRAGAGKPTIYR |
| | VP16 | WWTNKAALIAEVYENEIEQVRKFPDLGSFKADLDFLLHNLWKVWRETICGEAF |
| | | RCVIAEAQLDPVTLTQLKDQFMERRREIPKKLVEDAISNGELPKDINRELLLDMI |
| | | FGFCWYRLLTEQLTVEQDIEEFTFLLINGVCPGTQCSGGGGPTDALDDFDLDML |
| | | PADALDDFDLDMLPADALDDFDLDMLPGPPKKKRKV |
| 15 | cTA | MSPKRRTQAERAMETQGKLIAAALGVLREKGYAGFRIADVPGAAGVSRGAQS |
| | | HHFPTKLELLLATFEWLYEQITERSRARLAKLKPEDDVIQQMLDDAAEFFLDDD |
| | | FSIGLDLIVAADRDPALREGIQRTVERNRFVVEDMWLGVLVSRGLSRDDAEDIL |
| | | WLIFNSVRGLVVRSWQKDKERFERVRNSTLEIARERYAKFKRSGGGGPTDAL |
| | | DDFDLDMLPADALDDFDLDMLPADALDDFDLDMLPGPPKKKRKV |
| 16 | rcTA | MVIMSPKRRTQAERAMETQGKLIAAALGVLREKGYAGFRIADVPGAAGVSRG |
| | | AQSHHFPTKLELLLATFEWLYEQITERSRARLAKLKPEDDVIQQMLDDAAEFFL |
| | | DDDFSIGLDLIVAADRDPVLREGIQRTVERNRFVVGDIWLGVLVSRGLSRDDAE |
| | | DILWLIFNSVRGLVVRSLWQKDKERFERVRNSTLEIARERYAKFKRSGGGGPTD |
| | | ALDDFDLDMLPADALDDFDLDMLPADALDDFDLDMLPGPPKKKRKV |

TABLE 3
Exemplary AAV vector production components.

| SEQ | | |
|---|---|---|
| ID | | |
| NO: | Name | Sequence |
| 17 | WT Full | ATGCCGGGGTTTTACGAGATTGTGATTAAGGTCCCCAGCGACCTTGACGAGC |
| | Rep | ATCTGCCCGGCATTTCTGACAGCTTTGTGAACTGGGTGGCCGAGAAGGAATG |
| | | GGAGTTGCCGCCAGATTCTGACATGGATCTGAATCTGATTGAGCAGGCACCC |
| | | CTGACCGTGGCCGAGAAGCTGCAGCGCGACTTTCTGACGGAATGGCGCCGTG |
| | | TGAGTAAGGCCCCGGAGGCCCTTTTCTTTGTGCAATTTGAGAAGGGAGAGAG |
| | | CTACTTCCACATGCACGTGCTCGTGGAAACCACCGGGGTGAAATCCATGGTT |
| | | TTGGGACGTTTCCTGAGTCAGATTCGCGAAAAACTGATTCAGAGAATTTACC |
| | | GCGGGATCGAGCCGACTTTGCCAAACTGGTTCGCGGTCACAAAGACCAGAAA |
| | | TGGCGCCGGAGGCGGGAACAAGGTGGTGGATGAGTGCTACATCCCCAATTAC |
| | | TTGCTCCCCAAAACCCAGCCTGAGCTCCAGTGGGCGTGGACTAATATGGAAC |
| | | AGTATTTAAGCGCCTGTTTGAATCTCACGGAGCGTAAACGGTTGGTGGCGCA |
| | | GCATCTGACGCACGTGTCGCAGACGCAGGAGCAGAACAAAGAGAATCAGAA |
| | | TCCCAATTCTGATGCGCCGGTGATCAGATCAAAAACTTCAGCCAGGTACATG |
| | | GAGCTGGTCGGGTGGCTCGTGGACAAGGGGATTACCTCGGAGAAGCAGTGG |
| | | ATCCAGGAGGACCAGGCCTCATACATCTCCTTCAATGCGGCCTCCAACTCGC |
| | | GGTCCCAAATCAAGGCTGCCTTGGACAATGCGGGAAAGATTATGAGCCTGAC |
| | | TAAAACCGCCCCCGACTACCTGGTGGGCCAGCAGCCCGTGGAGGACATTTCC |
| | | AGCAATCGGATTTATAAAATTTTGGAACTAAACGGGTACGATCCCCAATATG |
| | | CGGCTTCCGTCTTTCTGGGATGGGCCACGAAAAAGTTCGGCAAGAGGAACAC |
| | | CATCTGGCTGTTTGGGCCTGCAACTACCGGGAAGACCAACATCGCGGAGGCC |
| | | ATAGCCCACACTGTGCCCTTCTACGGGTGCGTAAACTGGACCAATGAGAACT |
| | | TTCCCTTCAACGACTGTGTCGACAAGATGGTGATCTGGTGGGAGGAGGGGAA |
| | | GATGACCGCCAAGGTCGTGGAGTCGGCCAAAGCCATTCTCGGAGGAAGCAA |
| | | GGTGCGCGTGGACCAGAAATGCAAGTCCTCGGCCCAGATAGACCCGACTCCC |
| | | GTGATCGTCACCTCCAACACCAACATGTGCGCCGTGATTGACGGGAACTCAA |
| | | CGACCTTCGAACACCAGCAGCCGTTGCAAGACCGGATGTTCAAATTTGAACT |
| | | CACCCGCCGTCTGGATCATGACTTTGGGAAGGTCACCAAGCAGGAAGTCAAA |
| | | GACTTTTTCCGGTGGGCAAAGGATCACGTGGTTGAGGTGGAGCATGAATTCT |
| | | ACGTCAAAAAGGGTGGAGCCAAGAAAAGACCCGCCCCCAGTGACGCAGATA |
| | | TAAGTGAGCCCAAACGGGTGCGCGAGTCAGTTGCGCAGCCATCGACGTCAGA |
| | | CGCGGAAGCTTCGATCAACTACGCAGACAGGTACCAAAACAAATGTTCTCGT |
| | | CACGTGGGCATGAATCTGATGCTGTTTCCCTGCAGACAATGCGAGAGAATGA |
| | | ATCAGAATTCAAATATCTGCTTCACTCACGGACAGAAAGACTGTTTAGAGTG |
| | | CTTTCCCGTGTCAGAATCTCAACCCGTTTCTGTCGTCAAAAAGGCGTATCAGA |
| | | AACTGTGCTACATTCATCATATCATGGGAAAGGTGCCAGACGCTTGCACTGC |
| | | CTGCGATCTGGTCAATGTGGATTTGGATGACTGCATCTTTGAACAATAAATG |
| | | ATTTAAATCAGGTATGGCTGCCGATGGTTATCTTCCAGATTGGCTCGAGGAC |
| | | ACTCTCTCTGA |
| 18 | Rep52 | MELVGWLVDKGITSEKQWIQEDQASYISFNAASNSRSQIKAALDNAGKIMSLTK |
| | | TAPDYLVGQQPVEDISSNRIYKILELNGYDPQYAASVFLGWATKKFGKRNTIWLF |
| | | GPATTGKTNIAEAIAHTVPFYGCVNWTNENFPFNDCVDKMVIWWEEGKMTAKV |
| | | VESAKAILGGSKVRVDQKCKSSAQIDPTPVIVTSNTNMCAVIDGNSTTFEHQQPL |
| | | QDRMFKFELTRRLDHDFGKVTKQEVKDFFRWAKDHVVEVEHEFYVKKGGAKK |
| | | RPAPSDADISEPKRVRESVAQPSTSDAEASINYADRYQNKCSRHVGMNLMLFPC |
| | | RQCERMNQNSNICFTHGQKDCLECFPVSESQPVSVVKKAYQKLCYIHHIMGKVP |
| | | DACTACDLVNVDLDDCIFEQ |
| 19 | SS- | ATGGAATTAGTGGGCTGGTTGGTCGATAAAGGCATCACAAGCGAAAAACAA |
| | Rep52 | TGGATTCAAGAAGATCAAGCGAGCTATATTAGTTTTAACGCCGCTAGTAATA |
| | (splice | GCAGAAGTCAGATTAAAGCCGCTCTCGATAACGCCGGCAAAATCATGTCTTT |
| | mut) | AACCAAGACAGCTCCTGATTATTTAGTCGGGCAACAACCTGTCGAGGACATC |
| | | AGTTCTAACAGAATCTACAAGATCCTCGAATTGAATGGCTATGACCCTCAGT |
| | | ACGCCGCCAGTGTGTTCTTAGGCTGGGCTACCAAGAAATTTGGGAAACGCAA |
| | | TACAATTTGGTTATTCGGCCCCGCCACCACAGGCAAAACAAATATTGCCGAA |
| | | GCTATCGCTCATACCGTCCCTTTCTATGGCTGTGTGAATTGGACAAACGAAAA |
| | | TTTCCCTTTTAATGATTGCGTGGATAAAATGGTCATTTGGTGGGAAGAAGGCA |
| | | AAATGACAGCTAAAGTGGTCGAAAGCGCTAAGGCTATCTTGGGCGGCTCTAA |
| | | AGTCAGAGTCGATCAAAAGTGTAAAAGTAGCGCTCAAATCGATCCCACCCCT |
| | | GTCATTGTGACAAGTAATACAAATATGTGTGCTGTCATCGATGGCAATAGCA |
| | | CCACATTTGAGCATCAACAACCCCTCCAGGATAGAATGTTTAAGTTCGAGTT |
| | | GACAAGAAGATTAGACCACGATTTCGGCAAAGTGACAAAACAAGAGGTGAA |
| | | GGATTTCTTTAGATGGGCCAAAGACCATGTCGTGGAAGTCGAACACGAGTTT |
| | | TATGTGAAGAAAGGCGGCGCTAAAAAGCGGCCTGCTCCTTCCGATGCCGACA |
| | | TCTCCGAACCTAAGAGAGTCAGAGAAAGCGTGGCCCAACCCAGCACCAGCG |
| | | ATGCCGAGGCCAGCATTAATTATGCCGATCGCTATCAGAATAAGTGCAGCAG |
| | | ACATGTCGGGATGAACTTAATGTTATTCCCTTGTCGGCAGTGTGAACGGATG |
| | | AACCAAAACAGCAACATTTGTTTTACCCACGGACAAAAGGATTGCCTGGAAT |
| | | GTTTCCCTGTCAGCGAGAGCCAGCCTGTGAGCGTGGTGAAGAAAGCCTACCA |
| | | AAAGTTATGTTATATCCACCACATTATGGGCAAAGTCCCCGATGCCTGTACC |
| | | GCTTGTGACTTAGTGAACGTAGACCTCGACGATTGTATTTTCGAGCAGTGA |
| 20 | Rep40 | MELVGWLVDKGITSEKQWIQEDQASYISFNAASNSRSQIKAALDNAGKIMSLTK |
| | | TAPDYLVGQQPVEDISSNRIYKILELNGYDPQYAASVFLGWATKKFGKRNTIWLF |
| | | GPATTGKTNIAEAIAHTVPFYGCVNWTNENFPFNDCVDKMVIWWEEGKMTAKV |
| | | VESAKAILGGSKVRVDQKCKSSAQIDPTPVIVTSNTNMCAVIDGNSTTFEHQQPL |
| | | QDRMFKFELTRRLDHDFGKVTKQEVKDFFRWAKDHVVEVEHEFYVKKGGAKK |
| | | RPAPSDADISEPKRVRESVAQPSTSDAEASINYADRLARGHSL |
| 21 | Rep78 | MPGFYEIVIKVPSDLDEHLPGISDSFVNWVAEKEWELPPDSDMDLNLIEQAPLTV |
| | | AEKLQRDFLTEWRRVSKAPEALFFVQFEKGESYFHMHVLVETTGVKSMVLGRF |
| | | LSQIREKLIQRIYRGIEPTLPNWFAVTKTRNGAGGGNKVVDECYIPNYLLPKTQPE |
| | | LQWAWTNMEQYLSACLNLTERKRLVAQHLTHVSQTQEQNKENQNPNSDAPVIR |
| | | SKTSARYMELVGWLVDKGITSEKQWIQEDQASYISFNAASNSRSQIKAALDNAG |
| | | KIMSLTKTAPDYLVGQQPVEDISSNRIYKILELNGYDPQYAASVFLGWATKKFGK |
| | | RNTIWLFGPATTGKTNIAEAIAHTVPFYGCVNWTNENFPFNDCVDKMVIWWEE |
| | | GKMTAKVVESAKAILGGSKVRVDQKCKSSAQIDPTPVIVTSNTNMCAVIDGNST |
| | | TFEHQQPLQDRMFKFELTRRLDHDFGKVTKQEVKDFFRWAKDHVVEVEHEFYV |
| | | KKGGAKKRPAPSDADISEPKRVRESVAQPSTSDAEASINYADRYQNKCSRHVGM |
| | | NLMLFPCRQCERMNQNSNICFTHGQKDCLECFPVSESQPVSVVKKAYQKLCYIH |
| | | HIMGKVPDACTACDLVNVDLDDCIFEQ |
| 22 | SC/SS- | MPGFYEIVIKVPSDLDEHLPGISDSFVNWVAEKEWELPPDSDMDLNLIEQAPLTV |
| | Rep78 | AEKLQRDFLTEWRRVSKAPEALFFVQFEKGESYFHMHVLVETTGVKSMVLGRF |
| | (start + | LSQIREKLIQRIYRGIEPTLPNWFAVTKTRNGAGGGNKVVDECYIPNYLLPKTQPE |
| | splice) | LQWAWTNMEQYLSACLNLTERKRLVAQHLTHVSQTQEQNKENQNPNSDAPVIR |
| | | SKTSARYGELVGWLVDKGITSEKQWIQEDQASYISFNAASNSRSQIKAALDNAG |
| | | KIMSLTKTAPDYLVGQQPVEDISSNRIYKILELNGYDPQYAASVFLGWATKKFGK |
| | | RNTIWLFGPATTGKTNIAEAIAHTVPFYGCVNWTNENFPFNDCVDKMVIWWEE |
| | | GKMTAKVVESAKAILGGSKVRVDQKCKSSAQIDPTPVIVTSNTNMCAVIDGNST |
| | | TFEHQQPLQDRMFKFELTRRLDHDFGKVTKQEVKDFFRWAKDHVVEVEHEFYV |
| | | KKGGAKKRPAPSDADISEPKRVRESVAQPSTSDAEASINYADRYQNKCSRHVGM |
| | | NLMLFPCRQCERMNQNSNICFTHGQKDCLECFPVSESQPVSVVKKAYQKLCYIH |
| | | HIMGKVPDACTACDLVNVDLDDCIFEQ |
| 23 | | ATGCCTGGCTTCTACGAGATCGTGATCAAGGTGCCCAGCGACCTGGACGAGC |
| | | ATCTGCCTGGCATCAGCGACAGCTTCGTGAATTGGGTCGCCGAGAAAGAGTG |
| | | GGAGCTGCCTCCTGACAGCGACATGGACCTGAACCTGATTGAGCAGGCCCCT |
| | | CTGACAGTGGCCGAGAAGCTGCAGAGGGATTTCCTGACAGAGTGGCGGAGA |
| | | GTGTCTAAGGCCCCTGAGGCTCTGTTCTTCGTGCAGTTCGAGAAGGGCGAGA |
| | | GCTACTTCCACATGCACGTGCTGGTCGAGACAACCGGCGTGAAGTCTATGGT |
| | | GCTGGGCAGATTCCTGAGCCAGATCAGAGAGAAGCTGATCCAGCGGATCTAC |
| | | CGGGGCATCGAGCCCACACTGCCTAATTGGTTCGCCGTGACCAAGACCAGAA |
| | | ACGGTGCTGGCGGCGGAAACAAGGTGGTGGACGAGTGCTACATCCCCAACT |
| | | ACCTGCTGCCTAAGACACAGCCCGAACTGCAGTGGGCCTGGACCAACATGGA |
| | | ACAGTACCTGAGCGCCTGCCTGAATCTGACCGAGCGGAAAAGACTGGTGGCC |
| | | CAGCATCTGACCCACGTGTCCCAGACACAAGAGCAGAACAAAGAGAATCAG |
| | | AACCCCAACAGCGACGCCCCTGTGATCAGAAGCAAGACCAGCGCCAGATAC |
| | | GGaGAACTCGTTGGCTGGCTGGTGGACAAGGGCATCACAAGCGAGAAGCAGT |
| | | GGATCCAAGAGGACCAGGCCAGCTACATCAGCTTCAACGCCGCCTCCAACAG |
| | | CAGATCCCAGATCAAGGCCGCTCTGGACAACGCCGGCAAGATCATGAGCCTG |
| | | ACAAAGACAGCCCCTGACTACCTCGTGGGCCAGCAGCCTGTGGAAGATATCA |
| | | GCAGCAACCGGATCTACAAGATCCTGGAACTGAACGGCTACGACCCTCAGTA |
| | | TGCCGCCTCTGTGTTTCTCGGCTGGGCTACCAAGAAGTTCGGCAAGCGGAAC |
| | | ACCATCTGGCTGTTTGGCCCTGCCACAACCGGCAAGACCAATATCGCCGAGG |
| | | CTATCGCCCACACCGTGCCTTTTTACGGCTGCGTGAACTGGACCAATGAGAA |
| | | CTTCCCCTTCAACGACTGCGTGGACAAGATGGTCATTTGGTGGGAAGAGGGC |
| | | AAGATGACCGCCAAAGTGGTGGAAAGCGCCAAGGCCATCCTCGGCGGATCT |
| | | AAAGTTCGCGTGGACCAGAAGTGCAAGTCTAGCGCCCAGATCGACCCCACAC |
| | | CTGTGATCGTGACCAGCAACACCAACATGTGCGCCGTGATCGACGGCAACAG |
| | | CACCACCTTTGAACACCAGCAGCCACTGCAGGACCGGATGTTCAAGTTCGAG |
| | | CTGACCAGACGGCTGGACCACGACTTCGGCAAAGTGACCAAGCAAGAAGTG |
| | | AAGGACTTCTTCCGCTGGGCCAAAGATCACGTGGTGGAAGTGGAACACGAGT |
| | | TCTACGTGAAGAAAGGCGGAGCCAAGAAGAGGCCCGCTCCTTCCGATGCCG |
| | | ATATCAGCGAGCCTAAGCGCGTGCGGGAATCTGTGGCTCAGCCTAGCACATC |
| | | TGATGCCGAGGCCAGCATCAACTACGCCGACAGATACCAGAACAAGTGCAG |
| | | CCGGCACGTGGGAATGAATCTGATGCTGTTCCCCTGTCGGCAGTGCGAGCGG |
| | | ATGAACCAGAACAGCAACATCTGCTTCACCCACGGCCAGAAAGACTGCCTGG |
| | | AATGCTTCCCCGTGTCCGAGTCTCAGCCTGTGTCCGTGGTCAAGAAGGCCTAC |
| | | CAGAAGCTGTGTTACATCCACCACATCATGGGCAAAGTGCCCGATGCCTGCA |
| | | CCGCCTGCGATCTGGTTAATGTGGACCTGGATGACTGCATCTTCGAGCAGTG |
| | | A |
| 24 | Rep78 + | cTGGCGGGCTTCTACGAGATCGTGATCAAGGTGCCCAGCGACCTGGACGAGC |
| | Rep52 | ATCTGCCTGGCATCAGCGACAGCTTCGTGAATTGGGTCGCCGAGAAAGAGTG |
| | Only | GGAGCTGCCTCCTGACAGCGACITGGACCTGAACCTGATTGAGCAGGCCCCTC |
| | | TGACAGTGGCCGAGAAGCTGCAGAGGGATTTCCTGACAGAGTGGCGGAGAG |
| | | TGTCTAAGGCCCCTGAGGCTCTGTTCTTCGTGCAGTTCGAGAAGGGCGAGAG |
| | | CTACTTCCACTTACACGTGCTGGTCGAGACAACCGGCGTGAAGTCTTTAGTGC |
| | | TGGGCAGATTCCTGAGCCAGATCAGAGAGAAGCTGATCCAGCGGATCTACCG |
| | | GGGCATCGAGCCCACACTGCCTAATTGGTTCGCCGTGACCAAGACCAGAAAC |
| | | GGcGCTGGCGGCGGAAACAAGGTGGTGGACGAGTGCTACATCCCCAACTACC |
| | | TGCTGCCTAAGACACAGCCCGAACTGCAGTGGGCCTGGACCAACTTAGAACA |
| | | GTACCTGAGCGCCTGCCTGAATCTGACCGAGCGGAAAAGACTGGTGGCCCAG |
| | | CATCTGACCCACGTGTCCCAGACACAAGAGCAGAACAAAGAGAATCAGAAC |
| | | CCCAACAGCGACGCCCCTGTGATCAGAAGCAAGACCAGCGCCAGATACATG |
| | | GAACTCGTTGGCTGGCTGGTGGACAAGGGCATCACAAGCGAGAAGCAGTGG |
| | | ATCCAAGAGGACCAGGCCAGCTACATCAGCTTCAACGCCGCCTCCAACAGCA |
| | | GATCCCAGATCAAGGCCGCTCTGGACAACGCCGGCAAGATCATGAGCCTGAC |
| | | AAAGACAGCCCCTGACTACCTCGTGGGCCAGCAGCCTGTGGAAGATATCAGC |
| | | AGCAACCGGATCTACAAGATCCTGGAACTGAACGGCTACGACCCTCAGTATG |
| | | CCGCCTCTGTGTTTCTCGGCTGGGCTACCAAGAAGTTCGGCAAGCGGAACAC |
| | | CATCTGGCTGTTTGGCCCTGCCACAACCGGCAAGACCAATATCGCCGAGGCT |
| | | ATCGCCCACACCGTGCCTTTTTACGGCTGCGTGAACTGGACCAATGAGAACT |
| | | TCCCCTTCAACGACTGCGTGGACAAGATGGTCATTTGGTGGGAAGAGGGCAA |
| | | GATGACCGCCAAAGTGGTGGAAAGCGCCAAGGCCATCCTCGGCGGATCTAA |
| | | AGTTCGCGTGGACCAGAAGTGCAAGTCTAGCGCCCAGATCGACCCCACACCT |
| | | GTGATCGTGACCAGCAACACCAACATGTGCGCCGTGATCGACGGCAACAGCA |
| | | CCACCTTTGAACACCAGCAGCCACTGCAGGACCGGATGTTCAAGTTCGAGCT |
| | | GACCAGACGGCTGGACCACGACTTCGGCAAAGTGACCAAGCAAGAAGTGAA |
| | | GGACTTCTTCCGCTGGGCCAAAGATCACGTGGTGGAAGTGGAACACGAGTTC |
| | | TACGTGAAGAAAGGCGGAGCCAAGAAGAGGCCCGCTCCTTCCGATGCCGAT |
| | | ATCAGCGAGCCTAAGCGCGTGCGGGAATCTGTGGCTCAGCCTAGCACATCTG |
| | | ATGCCGAGGCCAGCATCAACTACGCCGACAGATACCAGAACAAGTGCAGCC |
| | | GGCACGTGGGAATGAATCTGATGCTGTTCCCCTGTCGGCAGTGCGAGCGGAT |
| | | GAACCAGAACAGCAACATCTGCTTCACCCACGGCCAGAAAGACTGCCTGGA |
| | | ATGCTTCCCCGTGTCCGAGTCTCAGCCTGTGTCCGTGGTCAAGAAGGCCTACC |
| | | AGAAGCTGTGTTACATCCACCACATCATGGGCAAAGTGCCCGATGCCTGCAC |
| | | CGCCTGCGATCTGGTTAATGTGGACCTGGATGACTGCATCTTCGAGCAGTGA |
| 25 | Rep52 | GCCGCCACCATGGAATTAGTGGGCTGGTTGGTCGATAAAGGCATCACAAGCG |
| | IRES | AAAAACAATGGATTCAAGAAGATCAAGCGAGCTATATTAGTTTTAACGCCGC |
| | Rep78 | TAGTAATAGCAGAAGTCAGATTAAAGCCGCTCTCGATAACGCCGGCAAAATC |
| | Only | ATGTCTTTAACCAAGACAGCTCCTGATTATTTAGTCGGGCAACAACCTGTCGA |
| | | GGACATCAGTTCTAACAGAATCTACAAGATCCTCGAATTGAATGGCTATGAC |
| | | CCTCAGTACGCCGCCAGTGTGTTCTTAGGCTGGGCTACCAAGAAATTTGGGA |
| | | AACGCAATACAATTTGGTTATTCGGCCCCGCCACCACAGGCAAAACAAATAT |
| | | TGCCGAAGCTATCGCTCATACCGTCCCTTTCTATGGCTGTGTGAATTGGACAA |
| | | ACGAAAATTTCCCTTTTAATGATTGCGTGGATAAAATGGTCATTTGGTGGGAA |
| | | GAAGGCAAAATGACAGCTAAAGTGGTCGAAAGCGCTAAGGCTATCTTGGGC |
| | | GGCTCTAAAGTCAGAGTCGATCAAAAGTGTAAAAGTAGCGCTCAAATCGATC |
| | | CCACCCCTGTCATTGTGACAAGTAATACAAATATGTGTGCTGTCATCGATGGC |
| | | AATAGCACCACATTTGAGCATCAACAACCCCTCCAGGATAGAATGTTTAAGT |
| | | TCGAGTTGACAAGAAGATTAGACCACGATTTCGGCAAAGTGACAAAACAAG |
| | | AGGTGAAGGATTTCTTTAGATGGGCCAAAGACCATGTCGTGGAAGTCGAACA |
| | | CGAGTTTTATGTGAAGAAAGGCGGCGCTAAAAAGCGGCCTGCTCCTTCCGAT |
| | | GCCGACATCTCCGAACCTAAGAGAGTCAGAGAAAGCGTGGCCCAACCCAGC |
| | | ACCAGCGATGCCGAGGCCAGCATTAATTATGCCGATCGCTATCAGAATAAGT |
| | | GCAGCAGACATGTCGGGATGAACTTAATGTTATTCCCTTGTCGGCAGTGTGA |
| | | ACGGATGAACCAAAACAGCAACATTTGTTTTACCCACGGACAAAAGGATTGC |
| | | CTGGAATGTTTCCCTGTCAGCGAGAGCCAGCCTGTGAGCGTGGTGAAGAAAG |
| | | CCTACCAAAAGTTATGTTATATCCACCACATTATGGGCAAAGTCCCCGATGC |
| | | CTGTACCGCTTGTGACTTAGTGAACGTAGACCTCGACGATTGTATTTTCGAGC |
| | | AGTGAtaaGcccctetecctcccccccccctaacgttactggccgaagccgcttggaataaggccggtgtgcgtttgt |
| | | ctatatgttattttccaccatattgccgtcttttggcaatgtgagggcccggaaacctggccctgtcttcttgacgag |
| | | cattcctaggggtctttcccctctcgccaaaggaatgcaaggtctgttgaatgtcgtgaaggaagcagttcctctgga |
| | | agcttcttgaagacaaacaacgtctgtagcgaccctttgcaggcagcggaaccccccacctggcgacaggtgcctctg |
| | | cggccaaaagccacgtgtataagatacacctgcaaaggcggcacaaccccagtgccacgttgtgagttggatagttgt |
| | | ggaaagagtcaaatggctctcctcaagcgtattcaacaaggggctgaaggatgcccagaaggtaccccattgtatggg |
| | | atctgatctggggcctcggtgcacatgctttacatgtgtttagtcgaggttaaaaaaacgtctaggccccccgaacca |
| | | cggggacgtggttttcctttgaaaaacacgatgataatatgCCTGGCTTCTACGAGATCGTGATCAAGGTGCCCAGCG |
| | | ACCTGGACGAGCATCTGCCTGGCATCAGCGACAGCTTCGTGAATTGGGTCGCCGAGAAAGAGTGGGAGCTG |
| | | CCTCCTGACAGCGACATGGACCTGAACCTGATTGAGCAGGCCCCTCTGACAG |
| | | TGGCCGAGAAGCTGCAGAGGGATTTCCTGACAGAGTGGCGGAGAGTGTCTA |
| | | AGGCCCCTGAGGCTCTGTTCTTCGTGCAGTTCGAGAAGGGCGAGAGCTACTT |
| | | CCACATGCACGTGCTGGTCGAGACAACCGGCGTGAAGTCTATGGTGCTGGGC |
| | | AGATTCCTGAGCCAGATCAGAGAGAAGCTGATCCAGCGGATCTACCGGGGC |
| | | ATCGAGCCCACACTGCCTAATTGGTTCGCCGTGACCAAGACCAGAAACGGTG |
| | | CTGGCGGCGGAAACAAGGTGGTGGACGAGTGCTACATCCCCAACTACCTGCT |
| | | GCCTAAGACACAGCCCGAACTGCAGTGGGCCTGGACCAACATGGAACAGTA |
| | | CCTGAGCGCCTGCCTGAATCTGACCGAGCGGAAAAGACTGGTGGCCCAGCAT |
| | | CTGACCCACGTGTCCCAGACACAAGAGCAGAACAAAGAGAATCAGAACCCC |
| | | AACAGCGACGCCCCTGTGATCAGAAGCAAGACCAGCGCCAGATACGGaGAA |
| | | CTCGTTGGCTGGCTGGTGGACAAGGGCATCACAAGCGAGAAGCAGTGGATCC |
| | | AAGAGGACCAGGCCAGCTACATCAGCTTCAACGCCGCCTCCAACAGCAGATC |
| | | CCAGATCAAGGCCGCTCTGGACAACGCCGGCAAGATCATGAGCCTGACAAA |
| | | GACAGCCCCTGACTACCTCGTGGGCCAGCAGCCTGTGGAAGATATCAGCAGC |
| | | AACCGGATCTACAAGATCCTGGAACTGAACGGCTACGACCCTCAGTATGCCG |
| | | CCTCTGTGTTTCTCGGCTGGGCTACCAAGAAGTTCGGCAAGCGGAACACCAT |
| | | CTGGCTGTTTGGCCCTGCCACAACCGGCAAGACCAATATCGCCGAGGCTATC |
| | | GCCCACACCGTGCCTTTTTACGGCTGCGTGAACTGGACCAATGAGAACTTCC |
| | | CCTTCAACGACTGCGTGGACAAGATGGTCATTTGGTGGGAAGAGGGCAAGAT |
| | | GACCGCCAAAGTGGTGGAAAGCGCCAAGGCCATCCTCGGCGGATCTAAAGTT |
| | | CGCGTGGACCAGAAGTGCAAGTCTAGCGCCCAGATCGACCCCACACCTGTGA |
| | | TCGTGACCAGCAACACCAACATGTGCGCCGTGATCGACGGCAACAGCACCAC |
| | | CTTTGAACACCAGCAGCCACTGCAGGACCGGATGTTCAAGTTCGAGCTGACC |
| | | AGACGGCTGGACCACGACTTCGGCAAAGTGACCAAGCAAGAAGTGAAGGAC |
| | | TTCTTCCGCTGGGCCAAAGATCACGTGGTGGAAGTGGAACACGAGTTCTACG |
| | | TGAAGAAAGGCGGAGCCAAGAAGAGGCCCGCTCCTTCCGATGCCGATATCA |
| | | GCGAGCCTAAGCGCGTGCGGGAATCTGTGGCTCAGCCTAGCACATCTGATGC |
| | | CGAGGCCAGCATCAACTACGCCGACAGATACCAGAACAAGTGCAGCCGGCA |
| | | CGTGGGAATGAATCTGATGCTGTTCCCCTGTCGGCAGTGCGAGCGGATGAAC |
| | | CAGAACAGCAACATCTGCTTCACCCACGGCCAGAAAGACTGCCTGGAATGCT |
| | | TCCCCGTGTCCGAGTCTCAGCCTGTGTCCGTGGTCAAGAAGGCCTACCAGAA |
| | | GCTGTGTTACATCCACCACATCATGGGCAAAGTGCCCGATGCCTGCACCGCC |
| | | TGCGATCTGGTTAATGTGGACCTGGATGACTGCATCTTCGAGCAGTGA |
| 26 | Rep68 | MPGFYEIVIKVPSDLDEHLPGISDSFVNWVAEKEWELPPDSDMDLNLIEQAPLTV |
| | | AEKLQRDFLTEWRRVSKAPEALFFVQFEKGESYFHMHVLVETTGVKSMVLGRF |
| | | LSQIREKLIQRIYRGIEPTLPNWFAVTKTRNGAGGGNKVVDECYIPNYLLPKTQPE |
| | | LQWAWTNMEQYLSACLNLTERKRLVAQHLTHVSQTQEQNKENQNPNSDAPVIR |
| | | SKTSARYMELVGWLVDKGITSEKQWIQEDQASYISFNAASNSRSQIKAALDNAG |
| | | KIMSLTKTAPDYLVGQQPVEDISSNRIYKILELNGYDPQYAASVFLGWATKKFGK |
| | | RNTIWLFGPATTGKTNIAEAIAHTVPFYGCVNWTNENFPFNDCVDKMVIWWEE |
| | | GKMTAKVVESAKAILGGSKVRVDQKCKSSAQIDPTPVIVTSNTNMCAVIDGNST |
| | | TFEHQQPLQDRMFKFELTRRLDHDFGKVTKQEVKDFFRWAKDHVVEVEHEFYV |
| | | KKGGAKKRPAPSDADISEPKRVRESVAQPSTSDAEASINYADRLARGHSL |
| 27 | SC- | MPGFYEIVIKVPSDLDEHLPGISDSFVNWVAEKEWELPPDSDMDLNLIEQAPLTV |
| | Rep68 | AEKLQRDFLTEWRRVSKAPEALFFVQFEKGESYFHMHVLVETTGVKSMVLGRF |
| | (start | LSQIREKLIQRIYRGIEPTLPNWFAVTKTRNGAGGGNKVVDECYIPNYLLPKTQPE |
| | mut) | LQWAWTNMEQYLSACLNLTERKRLVAQHLTHVSQTQEQNKENQNPNSDAPVIR |
| | | SKTSARYGELVGWLVDKGITSEKQWIQEDQASYISFNAASNSRSQIKAALDNAG |
| | | KIMSLTKTAPDYLVGQQPVEDISSNRIYKILELNGYDPQYAASVFLGWATKKFGK |
| | | RNTIWLFGPATTGKTNIAEAIAHTVPFYGCVNWTNENFPFNDCVDKMVIWWEE |
| | | GKMTAKVVESAKAILGGSKVRVDQKCKSSAQIDPTPVIVTSNTNMCAVIDGNST |
| | | TFEHQQPLQDRMFKFELTRRLDHDFGKVTKQEVKDFFRWAKDHVVEVEHEFYV |
| | | KKGGAKKRPAPSDADISEPKRVRESVAQPSTSDAEASINYADRLARGHSL |
| 28 | | ATGCCTGGCTTCTACGAGATCGTGATCAAGGTGCCCAGCGACCTGGACGAGC |
| | | ATCTGCCTGGCATCAGCGACAGCTTCGTGAATTGGGTCGCCGAGAAAGAGTG |
| | | GGAGCTGCCTCCTGACAGCGACATGGACCTGAACCTGATTGAGCAGGCCCCT |
| | | CTGACAGTGGCCGAGAAGCTGCAGAGGGATTTCCTGACAGAGTGGCGGAGA |
| | | GTGTCTAAGGCCCCTGAGGCTCTGTTCTTCGTGCAGTTCGAGAAGGGCGAGA |
| | | GCTACTTCCACATGCACGTGCTGGTCGAGACAACCGGCGTGAAGTCTATGGT |
| | | GCTGGGCAGATTCCTGAGCCAGATCAGAGAGAAGCTGATCCAGCGGATCTAC |
| | | CGGGGCATCGAGCCCACACTGCCTAATTGGTTCGCCGTGACCAAGACCAGAA |
| | | ACGGTGCTGGCGGCGGAAACAAGGTGGTGGACGAGTGCTACATCCCCAACT |
| | | ACCTGCTGCCTAAGACACAGCCCGAACTGCAGTGGGCCTGGACCAACATGGA |
| | | ACAGTACCTGAGCGCCTGCCTGAATCTGACCGAGCGGAAAAGACTGGTGGCC |
| | | CAGCATCTGACCCACGTGTCCCAGACACAAGAGCAGAACAAAGAGAATCAG |
| | | AACCCCAACAGCGACGCCCCTGTGATCAGAAGCAAGACCAGCGCCAGATAC |
| | | GGaGAACTCGTTGGCTGGCTGGTGGACAAGGGCATCACAAGCGAGAAGCAGT |
| | | GGATCCAAGAGGACCAGGCCAGCTACATCAGCTTCAACGCCGCCTCCAACAG |
| | | CAGATCCCAGATCAAGGCCGCTCTGGACAACGCCGGCAAGATCATGAGCCTG |
| | | ACAAAGACAGCCCCTGACTACCTCGTGGGCCAGCAGCCTGTGGAAGATATCA |
| | | GCAGCAACCGGATCTACAAGATCCTGGAACTGAACGGCTACGACCCTCAGTA |
| | | TGCCGCCTCTGTGTTTCTCGGCTGGGCTACCAAGAAGTTCGGCAAGCGGAAC |
| | | ACCATCTGGCTGTTTGGCCCTGCCACAACCGGCAAGACCAATATCGCCGAGG |
| | | CTATCGCCCACACCGTGCCTTTTTACGGCTGCGTGAACTGGACCAATGAGAA |
| | | CTTCCCCTTCAACGACTGCGTGGACAAGATGGTCATTTGGTGGGAAGAGGGC |
| | | AAGATGACCGCCAAAGTGGTGGAAAGCGCCAAGGCCATCCTCGGCGGATCT |
| | | AAAGTTCGCGTGGACCAGAAGTGCAAGTCTAGCGCCCAGATCGACCCCACAC |
| | | CTGTGATCGTGACCAGCAACACCAACATGTGCGCCGTGATCGACGGCAACAG |
| | | CACCACCTTTGAACACCAGCAGCCACTGCAGGACCGGATGTTCAAGTTCGAG |
| | | CTGACCAGACGGCTGGACCACGACTTCGGCAAAGTGACCAAGCAAGAAGTG |
| | | AAGGACTTCTTCCGCTGGGCCAAAGATCACGTGGTGGAAGTGGAACACGAGT |
| | | TCTACGTGAAGAAAGGCGGAGCCAAGAAGAGGCCCGCTCCTTCCGATGCCG |
| | | ATATCAGCGAGCCTAAGCGCGTGCGGGAATCTGTGGCTCAGCCTAGCACATC |
| | | TGATGCCGAGGCCAGCATCAACTACGCCGACAGACTGGCCAGGGGCCACAG |
| | | CCTGTGA |
| 29 | E2A | MASREEEQRETTPERGRGAARRPPTMEDVSSPSPSPPPPRAPPKKRLRRRLESEDE |
| | | EDSSQDALVPRTPSPRPSTSTADLAIASKKKKKRPSPKPERPPSPEVIVDSEEERED |
| | | VALQMVGFSNPPVLIKHGKGGKRTVRRLNEDDPVARGMRTQEEKEESSEAESES |
| | | TVINPLSLPIVSAWEKGMEAARALMDKYHVDNDLKANFKLLPDQVEALAAVCK |
| | | TWLNEEHRGLQLTFTSNKTFVTMMGRFLQAYLQSFAEVTYKHHEPTGCALWLH |
| | | RCAEIEGELKCLHGSIMINKEHVIEMDVTSENGQRALKEQSSKAKIVKNRWGRN |
| | | VVQISNTDARCCVHDAACPANQFSGKSCGMFFSEGAKAQVAFKQIKAFMQALY |
| | | PNAQTGHGHLLMPLRCECNSKPGHAPFLGRQLPKLTPFALSNAEDLDADLISDKS |
| | | VLASVHHPALIVFQCCNPVYRNSRAQGGGPNCDFKISAPDLLNALVMVRSLWSE |
| | | NFTELPRMVVPEFKWSTKHQYRNVSLPVAHSDARQNPFDF |
| 30 | VP1 | MAADGYLPDWLEDTLSEGIRQWWKLKPGPPPPKPAERHKDDSRGLVLPGYKYL |
| | | GPFNGLDKGEPVNEADAAALEHDKAYDRQLDSGDNPYLKYNHADAEFQERLK |
| | | EDTSFGGNLGRAVFQAKKRVLEPLGLVEEPVKTAPGKKRPVEHSPVEPDSSSGT |
| | | GKAGQQPARKRLNFGQTGDADSVPDPQPLGQPPAAPSGLGTNTMATGSGAPMA |
| | | DNNEGADGVGNSSGNWHCDSTWMGDRVITTSTRTWALPTYNNHLYKQISSQSG |
| | | ASNDNHYFGYSTPWGYFDFNRFHCHFSPRDWQRLINNNWGFRPKRLNFKLFNIQ |
| | | VKEVTQNDGTTTIANNLTSTVQVFTDSEYQLPYVLGSAHQGCLPPFPADVFMVP |
| | | QYGYLTLNNGSQAVGRSSFYCLEYFPSQMLRTGNNFTFSYTFEDVPFHSSYAHS |
| | | QSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFP |
| | | QSGVLIFGKQGSEKTNVDIEKVMITDEEEIRTTNPVATEQYGSVSTNLQRGNRQA |
| | | ATADVNTQGVLPGMVWQDRDVYLQGPIWAKIPHTDGHFHPSPLMGGFGLKHPP |
| | | PQILIKNTPVPANPSTTFSAAKFASFITQYSTGQVSVEIEWELQKENSKRWNPEIQ |
| | | YTSNYNKSVNVDFTVDTNGVYSEPRPIGTRYLTRNL |
| 31 | SC-VP1 | MAADGYLPDWLEDTLSEGIRQWWKLKPGPPPPKPAERHKDDSRGLVLPGYKYL |
| | (start | GPFNGLDKGEPVNEADAAALEHDKAYDRQLDSGDNPYLKYNHADAEFQERLK |
| | mut) | EDTSFGGNLGRAVFQAKKRVLEPLGLVEEPVKTAPGKKRPVEHSPVEPDSSSGT |
| | | GKAGQQPARKRLNFGQTGDADSVPDPQPLGQPPAAPSGLGTNTMATGSGAPMA |
| | | DNNEGADGVGNSSGNWHCDSTWMGDRVITTSTRTWALPTYNNHLYKQISSQSG |
| | | ASNDNHYFGYSTPWGYFDFNRFHCHFSPRDWQRLINNNWGFRPKRLNFKLFNIQ |
| | | VKEVTQNDGTTTIANNLTSTVQVFTDSEYQLPYVLGSAHQGCLPPFPADVFMVP |
| | | QYGYLTLNNGSQAVGRSSFYCLEYFPSQMLRTGNNFTFSYTFEDVPFHSSYAHS |
| | | QSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFP |
| | | QSGVLIFGKQGSEKTNVDIEKVMITDEEEIRTTNPVATEQYGSVSTNLQRGNRQA |
| | | ATADVNTQGVLPGMVWQDRDVYLQGPIWAKIPHTDGHFHPSPLMGGFGLKHPP |
| | | PQILIKNTPVPANPSTTFSAAKFASFITQYSTGQVSVEIEWELQKENSKRWNPEIQ |
| | | YTSNYNKSVNVDFTVDTNGVYSEPRPIGTRYLTRNL |
| 32 | | ATGGCCGCTGACGGCTACTTGCCCGACTGGTTGGAAGATACCTTGAGCGAGG |
| | | GCATCCGGCAATGGTGGAAATTGAAGCCCGGACCTCCACCTCCCAAACCAGC |
| | | CGAAAGACACAAAGATGATTCTCGCGGCTTGGTCTTGCCCGGCTATAAATAT |
| | | TTGGGCCCTTTTAATGGCTTGGATAAAGGCGAACCCGTGAATGAAGCCGATG |
| | | CTGCCGCTTTGGAACATGATAAGGCTTATGATAGACAATTGGATTCTGGCGA |
| | | TAATCCCTATTTGAAATATAATCATGCTGATGCCGAATTCCAAGAAAGATTG |
| | | AAGGAGGACACCAGCTTCGGCGGGAATTTGGGCAGGGCCGTGTTTCAAGCCA |
| | | AGAAACGCGTGTTGGAGCCCTTAGGGTTAGTGGAAGAGCCCGTGAAAACCG |
| | | CCCCTGGCAAGAAACGCCCCGTGGAACATAGCCCCGTCGAACCCGATAGTAG |
| | | TAGCGGCACAGGCAAAGCCGGGCAACAACCCGCCCGGAAGCGGCTCAACTT |
| | | CGGCCAAACCGGCGATGCCGATAGCGTGCCCGATCCTCAACCCTTGGGCCAA |
| | | CCACCCGCCGCTCCTAGCGGCTTAGGCACCAACACTATGGCCACCGGGTCCG |
| | | GTGCCCCTATGGCCGATAACAATGAAGGGGCTGATGGCGTCGGCAACAGTAG |
| | | CGGCAACTGGCACTGTGACAGTACCTGGATGGGTGATCGGGTGATTACAACA |
| | | TCTACAAGGACATGGGCTTTACCTACATATAATAATCATTTGTATAAGCAGAT |
| | | CAGTTCTCAGAGCGGCGCAAGCAATGATAACCATTATTTCGGGTATTCTACA |
| | | CCCTGGGGCTACTTCGATTTTAATCGGTTTCATTGTCATTTCAGCCCCAGAGA |
| | | TTGGCAGCGGTTGATTAATAATAATTGGGGCTTTAGGCCTAAACGGTTGAATT |
| | | TTAAATTGTTCAATATCCAGGTGAAGGAAGTGACCCAAAACGATGGCACCAC |
| | | CACCATCGCTAACAATTTGACATCTACCGTGCAAGTCTTCACCGATAGCGAA |
| | | TATCAATTGCCCTATGTGTTGGGGAGCGCCCACCAGGGCTGTTTGCCTCCCTT |
| | | TCCCGCCGATGTGTTTATGGTCCCGCAATACGGCTATTTGACATTAAATAATG |
| | | GCTCCCAAGCCGTGGGCAGAAGCAGCTTCTATTGTTTAGAATATTTCCCCAGC |
| | | CAAATGTTAAGAACAGGCAATAATTTCACATTCTCTTATACCTTCGAAGATGT |
| | | GCCCTTTCATTCTTCTTATGCCCATTCTCAATCCTTAGATAGATTGATGAACCC |
| | | CTTGATTGATCAATATTTATACTATCTCTCTCGGACCAATACCCCCTCCGGCA |
| | | CAACAACCCAAAGCCGCTTGCAATTCAGCCAAGCTGGCGCCTCCGATATCAG |
| | | AGATCAAAGCCGCAATTGGTTGCCCGGCCCTTGCTATAGACAACAAAGGGTG |
| | | AGCAAAACCAGCGCCGACAATAATAATTCCGAGTATAGCTGGACCGGCGCC |
| | | ACAAAATATCATTTGAACGGGCGGGATAGCTTAGTCAACCCCGGGCCCGCTA |
| | | TGGCCTCTCATAAAGATGACGAGGAGAAATTCTTCCCACAATCTGGCGTGTT |
| | | GATTTTCGGCAAACAGGGGAGCGAAAAGACCAACGTCGATATTGAGAAAGT |
| | | GATGATCACCGATGAGGAAGAGATTCGCACCACAAACCCTGTCGCCACCGAA |
| | | CAATACGGCAGCGTGAGCACAAATTTGCAACGGGGTAATCGGCAGGCCGCC |
| | | ACAGCCGACGTGAATACCCAGGGAGTGTTGCCCGGGATGGTGTGGCAAGATC |
| | | GGGACGTCTATTTGCAAGGCCCTATTTGGGCCAAAATCCCTCATACCGATGG |
| | | CCACTTCCATCCTAGCCCTTTGATGGGCGGCTTTGGCTTGAAGCATCCTCCGC |
| | | CCCAAATCTTGATTAAAAATACACCCGTGCCCGCCAACCCCAGCACAACATT |
| | | TTCCGCCGCCAAATTCGCCAGTTTCATTACCCAATATAGTACCGGCCAAGTGT |
| | | CTGTCGAAATTGAATGGGAATTACAAAAAGAGAATTCTAAGAGATGGAACC |
| | | CTGAGATCCAATATACCAGTAATTATAACAAAAGCGTGAACGTCGATTTCAC |
| | | CGTCGATACCAACGGGGTCTACAGCGAACCCAGACCTATCGGGACACGGTAT |
| | | TTAACCAGAAACTTATAG |
| 33 | VP2 | TAPGKKRPVEHSPVEPDSSSGTGKAGQQPARKRLNFGQTGDADSVPDPQPLGQP |
| | | PAAPSGLGTNTMATGSGAPMADNNEGADGVGNSSGNWHCDSTWMGDRVITTS |
| | | TRTWALPTYNNHLYKQISSQSGASNDNHYFGYSTPWGYFDFNRFHCHFSPRDW |
| | | QRLINNNWGFRPKRLNFKLFNIQVKEVTQNDGTTTIANNLTSTVQVFTDSEYQLP |
| | | YVLGSAHQGCLPPFPADVFMVPQYGYLTLNNGSQAVGRSSFYCLEYFPSQMLRT |
| | | GNNFTFSYTFEDVPFHSSYAHSQSLDRLMNPLIDQYLYYLSRTNTPSGTTTQSRL |
| | | QFSQAGASDIRDQSRNWLPGPCYRQQRVSKTSADNNNSEYSWTGATKYHLNGR |
| | | DSLVNPGPAMASHKDDEEKFFPQSGVLIFGKQGSEKTNVDIEKVMITDEEEIRTT |
| | | NPVATEQYGSVSTNLQRGNRQAATADVNTQGVLPGMVWQDRDVYLQGPIWAK |
| | | IPHTDGHFHPSPLMGGFGLKHPPPQILIKNTPVPANPSTTFSAAKFASFITQYSTGQ |
| | | VSVEIEWELQKENSKRWNPEIQYTSNYNKSVNVDFTVDTNGVYSEPRPIGTRYLT |
| | | RNL |
| 34 | VP3 | MATGSGAPMADNNEGADGVGNSSGNWHCDSTWMGDRVITTSTRTWALPTYN |
| | | NHLYKQISSQSGASNDNHYFGYSTPWGYFDFNRFHCHFSPRDWQRLINNNWGF |
| | | RPKRLNFKLFNIQVKEVTQNDGTTTIANNLTSTVQVFTDSEYQLPYVLGSAHQGC |
| | | LPPFPADVFMVPQYGYLTLNNGSQAVGRSSFYCLEYFPSQMLRTGNNFTFSYTFE |
| | | DVPFHSSYAHSQSLDRLMNPLIDQYLYYLSRTNTPSGTTTQSRLQFSQAGASDIR |
| | | DQSRNWLPGPCYRQQRVSKTSADNNNSEYSWTGATKYHLNGRDSLVNPGPAM |
| | | ASHKDDEEKFFPQSGVLIFGKQGSEKTNVDIEKVMITDEEEIRTTNPVATEQYGS |
| | | VSTNLQRGNRQAATADVNTQGVLPGMVWQDRDVYLQGPIWAKIPHTDGHFHP |
| | | SPLMGGFGLKHPPPQILIKNTPVPANPSTTFSAAKFASFITQYSTGQVSVEIEWELQ |
| | | KENSKRWNPEIQYTSNYNKSVNVDFTVDTNGVYSEPRPIGTRYLTRNL |
| 35 | E4 ORF6 | MTTSGVPFGMTLRPTRSRLSRRTPYSRDRLPPFETETRATILEDHPLLPECNTLTM |
| | | HNVSYVRGLPCSVGFTLIQEWVVPWDMVLTREELVILRKCMHVCLCCANIDIMT |
| | | SMMIHGYESWALHCHCSSPGSLQCIAGGQVLASWFRMVVDGAMFNQRFIWYRE |
| | | VVNYNMPKEVMFMSSVFMRGRHLIYLRLWYDGHVGSVVPAMSFGYSALHCGI |
| | | LNNIVVLCCSYCADLSEIRVRCCARRTRRLMLRAVRIIAEETTAMLYSCRTERRR |
| | | QQFIRALLQHHRPILMHDYDSTPM |
| 36 | SS-E4 | ATGACTACGTCCGGCGTTCCATTTGGCATGACACTACGACCAACACGATCTC |
| | ORF6 | GGTTGTCTCGGCGCACTCCGTACAGTAGGGATCGCCTACCTCCTTTTGAGACA |
| | (splice | GAGACCCGCGCTACCATACTGGAGGATCATCCGCTGCTGCCCGAATGTAACA |
| | site | CTTTGACAATGCACAATGTTTCCTACGTGCGAGGTCTTCCCTGCAGTGTGGGA |
| | removed) | TTTACGCTGATTCAGGAATGGGTTGTTCCCTGGGATATGGTTCTGACGCGGGA |
| | | GGAGCTTGTAATCCTGAGGAAGTGTATGCACGTGTGCCTGTGTTGTGCCAAC |
| | | ATTGATATCATGACGAGCATGATGATCCATGGTTACGAGTCCTGGGCTCTCC |
| | | ACTGTCATTGTTCCAGTCCCGGTTCCCTGCAGTGCATAGCCGGCGGGCAGGTT |
| | | TTGGCCAGCTGGTTTAGGATGGTGGTGGATGGCGCCATGTTTAATCAGAGGT |
| | | TTATATGGTACCGGGAGGTGGTGAATTACAACATGCCAAAAGAGGTAATGTT |
| | | TATGTCCAGCGTGTTTATGAGGGGTCGCCACTTAATCTACCTGCGCTTGTGGT |
| | | ATGATGGCCACGTGGGTTCTGTGGTCCCCGCCATGAGCTTTGGATACAGCGC |
| | | CTTGCACTGTGGGATTTTGAACAATATTGTGGTGCTGTGCTGCAGTTACTGTG |
| | | CTGATTTAAGTGAGATCAGGGTGCGCTGCTGTGCCCGGAGGACAAGGCGTCT |
| | | CATGCTGCGGGCGGTGCGAATCATCGCTGAGGAGACCACTGCCATGTTGTAT |
| | | TCCTGCAGGACGGAGCGGCGGCGGCAGCAGTTTATTCGCGCGCTGCTGCAGC |
| | | ACCACCGCCCTATCCTGATGCACGATTATGACTCTACCCCCATGTAGTAA |
| 37 | AAP | LETQTQYLTPSLSDSHQQPPLVWELIRWLQAVAHQWQTITRAPTEWVIPREIGIAI |
| | | PHGWATESSPPAPEPGPCPPTTTTSTNKFPANQEPRTTITTLATAPLGGILTSTDST |
| | | ATFHHVTGKDSSTTTGDSDPRDSTSSSLTFKSKRSRRMTVRRRLPITLPARFRCLL |
| | | TRSTSSRTSSARRIKDASRRSQQTSSWCHSMDTSP |
| 38 | VA RNA | CGACGTAATCCGTAGATGTACCTGGACATCCAGGTGATGCCGGCGGCGGTGG |
| | | TGGAGGCGCGCGGAAAGTCGCGGACGCGGTTCCAGATGTTGCGCAGCGGCA |
| | | AAAAGTGCTCCATGGTCGGGACGCTCTGGCCGGTGAGGCGTGCGCAGTCGTT |
| | | GACGCTCTAGACCGTGCAAAAGGAGAGCCTGTAAGCGGGCACTCTTCCGTGG |
| | | TCTGGTGGATAAATTCGCAAGGGTATCATGGCGGACGACCGGGGTTCGAACC |
| | | CCGGATCCGGCCGTCCGCCGTGATCCATGCGGTTACCGCCCGCGTGTCGAAC |
| | | CCAGGTGTGCGACGTCAGACAACGGGGGAGCGCTCCTTTTGGCTTCCTTCCA |
| | | GGCGCGGCGGCTGCTGCGCTAGCTTTTTTGGCCACTGGCCGCGCGCGGCGTA |
| | | AGCGGTTAGGCTGGAAAGCGAAAGCATTAAGTGGCTCGCTCCCTGTAGCCGG |
| | | AGGGTTATTTTCCAAGGGTTGAGTCGCAGGACCCCCGGTTCGAGTCTCGGGC |
| | | CGGCCGGACTGCGGCGAACGGGGGTTTGCCTCCCCGTCATGCAAGACCCCGC |
| | | TTGCAAATTCCTCCGGAAACAGGGACGAGCCCCTTTTTTGCTTTTCCCAGATG |
| | | CATCCGGTGCTGCGGCAGATGCGCCCCCCTCCTCAGCAGCGGCAAGAGCAAG |
| | | AGCAGCGGCAGACATGCAGGGCACCCTCCCCTTCTCCTACCGCGTCAGGAGG |
| | | GGCAACATCC |
| 39 | UL30 | MFSGGGGPLSPGGKSAARAASGFFAPAGPRGASRGPPPCLRQNFYNPYLAPVGT |
| | | QQKPTGPTQRHTYYSECDEFRFIAPRVLDEDAPPEKRAGVHDGHLKRAPKVYCG |
| | | GDERDVLRVGSGGFWPRRSRLWGGVDHAPAGFNPTVTVFHVYDILENVEHAYG |
| | | MRAAQFHARFMDAITPTGTVITLLGLTPEGHRVAVHVYGTRQYFYMNKEEVDR |
| | | HLQCRAPRDLCERMAAALRESPGASFRGISADHFEAEVVERTDVYYYETRPALF |
| | | YRVYVRSGRVLSYLCDNFCPAIKKYEGGVDATTRFILDNPGFVTFGWYRLKPGR |
| | | NNTLAQPRAPMAFGTSSDVEFNCTADNLAIEGGMSDLPAYKLMCFDIECKAGGE |
| | | DELAFPVAGHPEDLVIQISCLLYDLSTTALEHVLLFSLGSCDLPESHLNELAARGL |
| | | PTPVVLEFDSEFEMLLAFMTLVKQYGPEFVTGYNIINFDWPFLLAKLTDIYKVPL |
| | | DGYGRMNGRGVFRVWDIGQSHFQKRSKIKVNGMVNIDMYGIITDKIKLSSYKLN |
| | | AVAEAVLKDKKKDLSYRDIPAYYAAGPAQRGVIGEYCIQDSLLVGQLFFKFLPH |
| | | LELSAVARLAGINITRTIYDGQQIRVFTCLLRLADQKGFILPDTQGRFRGAGGEAP |
| | | KRPAAAREDEERPEEEGEDEDEREEGGGEREPEGARETAGRHVGYQGARVLDPT |
| | | SGFHVNPVVVFDFASLYPSIIQAHNLCFSTLSLRADAVAHLEAGKDYLEIEVGGR |
| | | RLFFVKAHVRESLLSILLRDWLAMRKQIRSRIPQSSPEEAVLLDKQQAAIKVVCN |
| | | SVYGFTGVQHGLLPCLHVAATVTTIGREMLLATREYVHARWAAFEQLLADFPE |
| | | AADMRAPGPYSMRIIYGDTDSIFVLCRGLTAAGLTAVGDKMASHISRALFLPPIK |
| | | LECEKTFTKLLLIAKKKYIGVIYGGKMLIKGVDLVRKNNCAFINRTSRALVDLLF |
| | | YDDTVSGAAAALAERPAEEWLARPLPEGLQAFGAVLVDAHRRITDPERDIQDFV |
| | | LTAELSRHPRAYTNKRLAHLTVYYKLMARRAQVPSIKDRIPYVIVAQTREVEET |
| | | VARLAALRELDAAAPGDEPAPPAALPSPAKRPRETPSPADPPGGASKPRKLLVSE |
| | | LAEDPAYAIAHGVALNTDYYFSHLLGAACVTFKALFGNNAKITESLLKRFIPEVW |
| | | HPPDDVAARLRTAGFGAVGAGATAEETRRMLHRAFDTLA |
| 40 | UL42 | MTDSPGGVAPASPVEDASDASLGQPEEGAPCQVVLQGAELNGILQAFAPLRTSL |
| | | LDSLLVMGDRGILIHNTIFGEQVFLPLEHSQFSRYRWRGPTAAFLSLVDQKRSLLS |
| | | VFRANQYPDLRRVELAITGQAPFRTLVQRIWTTTSDGEAVELASETLMKRELTSF |
| | | VVLVPQGTPDVQLRLTRPQLTKVLNATGADSATPTTFELGVNGKFSVFTTSTCVT |
| | | FAAREEGVSSSTSTQVQILSNALTKAGQAAANAKTVYGENTHRTFSVVVDDCSM |
| | | RAVLRRLQVGGGTLKFFLTTPVPSLCVTATGPNAVSAVFLLKPQKICLDWLGHS |
| | | QGSPSAGSSASRASGSEPTDSQDSASDAVSHGDPEDLDGAARAGEAGALHACPM |
| | | PSSTTRVTPTTKRGRSGGEDARADTALKKPKTGSPTAPPPADPVPLDTEDDSDAA |
| | | DGTAARPAAPDARSGSRYACYFRDLPTGEASPGAFSAFRGGPQTPYGFGFP |
| 41 | UL5 | MAAAGGERQLDGQKPGPPHLQQPGDRPAVPGRAEAFLNFTSMHGVQPILKRIRE |
| | | LSQQQLDGAQVPHLQWFRDVAALESPAGLPLREFPFAVYLITGNAGSGKSTCVQ |
| | | TINEVLDCVVTGATRIAAQNMYAKLSGAFLSRPINTIFHEFGFRGNHVQAQLGQY |
| | | PYTLTSNPASLEDLQRRDLTYYWEVILDLTKRALAASGGEELRNEFRALAALERT |
| | | LGLAEGALTRLAPATHGALPAFTRSNVIVIDEAGLLGRHLLTAVVYCWWMINAL |
| | | YHTPQYAARLRPVLVCVGSPTQTASLESTFEHQKLRCSVRQSENVLTYLICNRTL |
| | | REYARLSYSWAIFINNKRCVEHEFGNLMKVLEYGLPITEEHMQFVDRFVVPENYI |
| | | TNPANLPGWTRLFSSHKEVSAYMAKLHAYLKVTREGEFVVFTLPVLTFVSVKEF |
| | | DEYRRLTHQPGLTIEKWLTANASRITNYSQSQDQDAGHMRCEVHSKQQLVVAR |
| | | NDVTYVLNSQIAVTARLRKLVFGFSGTFRAFEAVLRDDSFVKTQGETSVEFAYRF |
| | | LSRLIFSGLISFYNFLQRPGLDATQRTLAYARMGELTAEILSLRPKSSGVPTQASV |
| | | MADAGAPGERAFDFKQLGPRDGGPDDFPDDDLDVIFAGLDEQQLDVFYCHYTP |
| | | GEPETTAAVHTQFALLKRAFLGRFRILQELFGEAFEVAPFSTYVDNVIFRGCEML |
| | | TGSPRGGLMSVALQTDNYTLMGYTYARVFAFADELRRRHATANVAELLEEAPL |
| | | PYVVLRDQHGFMSVVNTNISEFVESIDSTELAMAINADYGISSKLAMTITRSQGLS |
| | | LDKVAICFTPGNLRLNSAYVAMSRTTSSEFLRMNLNPLRERHERDDVISEHILSAL |
| | | RDPNVVIVY |
| 42 | UL8 | MDTADIVWVEESVSAITLYAVWLPPRAREYFHALVYFVCRNAAGEGRARFAEV |
| | | SVTATELRDFYGSADVSVQAVVAAARAATTPAASPLEPLENPTLWRALYACVL |
| | | AALERQTGPVALFAPLRIGSDPRTGLVVKVERASWGPPAAPRAALLVAEANIDID |
| | | PMALAARVAEHPDARLAWARLAAIRDTPQCASAASLTVNITTGTALFAREYQTL |
| | | AFPPIKKEGAFGDLVEVCEVGLRPRGHPQRVTARVLLPRDYDYFVSAGEKFSAP |
| | | ALVALFRQWHTTVHAAPGALAPVFAFLGPEFEVRGGPVPYFAVLGFPGWPTFTV |
| | | PATAESARDLVRGAAAAYAALLGAWPAVGARVVLPPRAWPGVASAAAGCLLP |
| | | AVREAVARWHPATKIIQLLDPPAAVGPVWTARFCFPGLRAQLLAALADLGGSGL |
| | | ADPHGRTGLARLDALVVAAPSEPWAGAVLERLVPDTCNACPALRQLLGGVMA |
| | | AVCLQIEETASSVKFAVCGGDGGAFWGVFNVDPQDADAASGVIEDARRAIETAV |
| | | GAVLRANGLRLRHPLCLALEGVYTHAVAWSQAGVWFWNSRDNTDHLGGFPLR |
| | | GPAYTTAAGVVRDTLRRVLGLTTACVPEEDALTARGLMEDACDRLILDAFNKR |
| | | LDAEYWSVRVSPFEASDPLPPTAFRGGALLDAEHYWRRVVRVCPGGGESVGVP |
| | | VDLYPRPLVLPPVDCAHHLREILREIELVFTGVLAGVWGEGGKFVYPFDDKMSF |
| | | LFA |
| 43 | UL52 | MGQEDGNRGERRAAGTPVEVTALYATDGCVITSSIALLTNSLLGAEPVYIFSYDA |
| | | YTHDGRADGPTEQDRFEESRALYQASGGLNGDSFRVTFCLLGTEVGGTHQARG |
| | | RTRPMFVCRFERADDVAALQDALAHGTPLQPDHIAATLDAEATFALHANMILAL |
| | | TVAINNASPRTGRDAAAAQYDQGASLRSLVGRTSLGQRGLTTLYVHHEVRVLA |
| | | AYRRAYYGSAQSPFWFLSKFGPDEKSLVLTTRYYLLQAQRLGGAGATYDLQAIK |
| | | DICATYAIPHAPRPDTVSAASLTSFAAITRFCCTSQYARGAAAAGFPLYVERRIAA |
| | | DVRETSALEKFITHDRSCLRVSDREFITYIYLAHFECFSPPRLATHLRAVTTHDPNP |
| | | AASTEQPSPLGREAVEQFFCHVRAQLNIGEYVKHNVTPRETVLDGDTAKAYLRA |
| | | RTYAPGALTPAPAYCGAVDSATKMMGRLADAEKLLVPRGWPAFAPASPGEDTA |
| | | GGTPPPQTCGIVKRLLRLAATEQQGPTPPAIAALIRNAAVQTPLPVYRISMVPTGQ |
| | | AFAALAWDDWARITRDARLAEAVVSAEAAAHPDHGALGRRLTDRIRAQGPVM |
| | | PPGGLDAGGQMYVNRNEIFNGALAITNIILDLDIALKEPVPFRRLHEALGHFRRG |
| | | ALAAVQLLFPAARVDPDAYPCYFFKSACRPGPASVGSGSGLGNDDDGDWFPCY |
| | | DDAGDEEWAEDPGAMDTSHDPPDDEVAYFDLCHEVGPTAEPRETDSPVCSCTD |
| | | KIGLRVCMPVPAPYVVHGSLTMRGVARVIQQAVLLDRDFVEAIGSYVKNFLLID |
| | | TGVYAHGHSLRLPYFAKIAPDGPACGRLLPVFVIPPACKDVPAFVAAHADPRRFH |
| | | FHAPPTYLASPREIRVLHSLGGDYVSFFERKASRNALEHFGRRETLTEVLGRYNV |
| | | QPDAGGTVEGFASELLGRIVACIETHFPEHAGEYQAVSVRRAVSKDDWVLLQLV |
| | | PVRGTLQQSLSCLRFKHGRASRATARTFVALSVGANNRLCVSLCQQCFAAKCDS |
| | | NRLHTLFTIDAGTPCSPSVPCSTSQPSS |
| 44 | UL29 | METKPKTATTIKVPPGPLGYVYARACPSEGIELLALLSARSGDSDVAVAPLVVGL |
| | | TVESGFEANVAVVVGSRTTGLGGTAVSLKLTPSHYSSSVYVFHGGRHLDPSTQA |
| | | PNLTRLCERARRHFGFSDYTPRPGDLKHETTGEALCERLGLDPDRALLYLVVTE |
| | | GFKEAVCINNTFLHLGGSDKVTIGGAEVHRIPVYPLQLFMPDFSRVIAEPFNANH |
| | | RSIGENFTYPLPFFNRPLNRLLFEAVVGPAAVALRCRNVDAVARAAAHLAFDEN |
| | | HEGAALPADITFTAFEASQGKTPRGGRDGGGKGPAGGFEQRLASVMAGDAALA |
| | | LESIVSMAVFDEPPTDISAWPLFEGQDTAAARANAVGAYLARAAGLVGAMVFST |
| | | NSALHLTEVDDAGPADPKDHSKPSFYRFFLVPGTHVAANPQVDREGHVVPGFEG |
| | | RPTAPLVGGTQEFAGEHLAMLCGFSPALLAKMLFYLERCDGGVIVGRQEMDVF |
| | | RYVADSNQTDVPCNLCTFDTRHACVHTTLMRLRARHPKFASAARGAIGVFGTM |
| | | NSMYSDCDVLGNYAAFSALKRADGSETARTIMQETYRAATERVMAELETLQYV |
| | | DQAVPTAMGRLETIITNREALHTVVNNVRQVVDREVEQLMRNLVEGRNFKFRD |
| | | GLGEANHAMSLTLDPYACGPCPLLQLLGRRSNLAVYQDLALSQCHGVFAGQSV |
| | | EGRNFRNQFQPVLRRRVMDMFNNGFLSAKTLTVALSEGAAICAPSLTAGQTAPA |
| | | ESSFEGDVARVTLGFPKELRVKSRVLFAGASANASEAAKARVASLQSAYQKPDK |
| | | RVDILLGPLGFLLKQFHAAIFPNGKPPGSNQPNPQWFWTALQRNQLPARLLSRED |
| | | IETIAFIKKFSLDYGAINFINLAPNNVSELAMYYMANQILRYCDHSTYFINTLTAII |
| | | AGSRRPPSVQAAAAWSAQGGAGLEAGARALMDAVDAHPGAWTSMFASCNLLR |
| | | PVMAARPMVVLGLSISKYYGMAGNDRVFQAGNWASLMGGKNACPLLIFDRTR |
| | | KFVLACPRAGFVCAASSLGGGAHESSLCEQLRGIISEGGAAVASSVFVATVKSLG |
| | | PRTQQLQIEDWLALLEDEYLSEEMMELTARALERGNGEWSTDAALEVAHEAEA |
| | | LVSQLGNAGEVFNFGDFGCEDDNATPFGGPGAPGPAFAGRKRAFHGDDPFGEGP |
| | | PDKKGDLTLDML |
| 49 | MAAP | LAHHHQSPQSGIRTTAGVLCFLGTSTSDPSTDSTRESRSTRQTPRPSSTTKPTTGSS |
| | | TAETTRTSSTTTPTRSFRSALKKIRLLGATSDEQSSRRKRGFLNLWAWLRNLLRR |
| | | LREKRGR |
| 50 | UL12 | MESTVGPACPPGRTVTKRPWALAEDTPRGPDSPPKRPRPNSLPLTTTFRPLPPPPQ |
| | | TTSAVDPSSHSPVNPPRDQHATDTADEKPRAASPALSDASGPPTPDIPLSPGGTHA |
| | | RDPDADPDSPDLDSMWSASVIPNALPSHILAETFERHLRGLLRGVRAPLAIGPLW |
| | | ARLDYLCSLAVVLEEAGMVDRGLGRHLWRLTRRGPPAAADAVAPRPLMGFYE |
| | | AATQNQADCQLWALLRRGLTTASTLRWGPQGPCFSPQWLKHNASLRPDVQSSA |
| | | VMFGRVNEPTARSLLFRYCVGRADDGGEAGADTRRFIFHEPSDLAEENVHTCGV |
| | | LMDGHTGMVGASLDILVCPRDIHGYLAPVPKTPLAFYEVKCRAKYAFDPMDPS |
| | | DPTASAYEDLMAHRSPEAFRAFIRSIPKPSVRYFAPGRVPGPEEALVTQDQAWSE |
| | | AHASGEKRRCSAADRALVELNSGVVSEVLLFGAPDLGRHTISPVSWSSGDLVRR |
| | | EPVFANPRHPNFKQILVQGYVLDSHFPDCPPHPHLVTFIGRHRTSAEEGVTFRLED |
| | | GAGALGAAGPSKASILPNQAVPIALIITPVRIDPEIYKAIQRSSRLAFDDTLAELWA |
| | | SRSPGPGPAAAETTSSSPTTGRSSR |
| 51 | ICP0 | MEPRPGASTRRPEGRPQREPAPDVWVFPCDRDLPDSSDSEAETEVGGRGDADHH |
| | | DDDSASEADSTDTELFETGLLGPQGVDGGAVSGGSPPREEDPGSCGGAPPREDG |
| | | GSDEGDVCAVCTDEIAPHLRCDTFPCMHRFCIPCMKTWMQLRNTCPLCNAKLV |
| | | YLIVGVTPSGSFSTIPIVNDPQTRMEAEEAVRAGTAVDFIWTGNQRFAPRYLTLG |
| | | GHTVRALSPTHPEPTTDEDDDDLDDADYVPPAPRRTPRAPPRRGAAAPPVTGGA |
| | | SHAAPQPAAARTAPPSAPIGPHGSSNTNTTTNSSGGGGSRQSRAAAPRGASGPSG |
| | | GVGVGVGVVEAEAGRPRGRTGPLVNRPAPLANNRDPIVISDSPPASPHRPPAAPM |
| | | PGSAPRPGPPASAAASGPARPRAAVAPCVRAPPPGPGPRAPAPGAEPAARPADAR |
| | | RVPQSHSSLAQAANQEQSLCRARATVARGSGGPGVEGGHGPSRGAAPSGAAPLP |
| | | SAASVEQEAAVRPRKRRGSGQENPSPQSTRPPLAPAGAKRAATHPPSDSGPGGR |
| | | GQGGPGTPLTSSAASASSSSASSSSAPTPAGAASSAAGAASSSASASSGGAVGAL |
| | | GGRQEETSLGPRAASGPRGPRKCARKTRHAETSGAVPAGGLTRYLPISGVSSVV |
| | | ALSPYVNKTITGDCLPILDMETGNIGAYVVLVDQTGNMATRLRAAVPGWSRRTL |
| | | LPETAGNHVMPPEYPTAPASEWNSLWMTPVGNMLFDQGTLVGALDFRSLRSRH |
| | | PWSGEQGASTRDEGKQ |
| 52 | ICP4 | MASENKQRPGSPGPTDGPPPTPSPDRDERGALGWGAETEEGGDDPDHDPDHPHD |
| | | LDDARRDGRAPAAGTDAGEDAGDAVSPRQLALLASMVEEAVRTIPTPDPAASPP |
| | | RTPAFRADDDDGDEYDDAADAAGDRAPARGREREAPLRGAYPDPTDRLSPRPP |
| | | AQPPRRRRHGRWRPSASSTSSDSGSSSSSSASSSSSSSDEDEDDDGNDAADHARE |
| | | ARAVGRGPSSAAPAAPGRTPPPPGPPPLSEAAPKPRAAARTPAASAGRIERRRAR |
| | | AAVAGRDATGRFTAGQPRRVELDADATSGAFYARYRDGYVSGEPWPGAGPPPP |
| | | GRVLYGGLGDSRPGLWGAPEAEEARRRFEASGAPAAVWAPELGDAAQQYALIT |
| | | RLLYTPDAEAMGWLQNPRVVPGDVALDQACFRISGAARNSSSFITGSVARAVPH |
| | | LGYAMAAGRFGWGLAHAAAAVAMSRRYDRAQKGFLLTSLRRAYAPLLARENA |
| | | ALTGAAGSPGAGADDEGVAAVAAAAPGERAVPAGYGAAGILAALGRLSAAPAS |
| | | PAGGDDPDAARHADADDDAGRRAQAGRVAVECLAACRGILEALAEGFDGDLA |
| | | AVPGLAGARPASPPRPEGPAGPASPPPPHADAPRLRAWLRELRFVRDALVLMRL |
| | | RGDLRVAGGSEAAVAAVRAVSLVAGALGPALPRDPRLPSSAAAAAADLLFDNQ |
| | | SLRPLLAAAASAPDAADALAAAAASAAPREGRKRKSPGPARPPGGGGPRPPKTK |
| | | KSGADAPGSDARAPLPAPAPPSTPPGPEPAPAQPAAPRAAAAQARPRPVAVSRRP |
| | | AEGPDPLGGWRRQPPGPSHTAAPAAAALEAYCSPRAVAELTDHPLFPVPWRPAL |
| | | MFDPRALASIAARCAGPAPAAQAACGGGDDDDNPHPHGAAGGRLFGPLRASGP |
| | | LRRMAAWMRQIPDPEDVRVVVLYSPLPGEDLAGGGASGGPPEWSAERGGLSCL |
| | | LAALANRLCGPDTAAWAGNWTGAPDVSALGAQGVLLLSTRDLAFAGAVEFLG |
| | | LLASAGDRRLIVVNTVRACDWPADGPAVSRQHAYLACELLPAVQCAVRWPAA |
| | | RDLRRTVLASGRVFGPGVFARVEAAHARLYPDAPPLRLCRGGNVRYRVRTRFGP |
| | | DTPVPMSPREYRRAVLPALDGRAAASGTTDAMAPGAPDFCEEEAHSHAACARW |
| | | GLGAPLRPVYVALGREAVRAGPARWRGPRRDFCARALLEPDDDAPPLVLRGDD |
| | | DGPGALPPAPPGIRWASATGRSGTVLAAAGAVEVLGAEAGLATPPRREVVDWE |
| | | GAWDEDDGGAFEGDGVL |
| 53 | ICP22 | MADISPGAFAPCVKARRPALRSPPLGTRKRKRPSRPLSSESEVESDTALESEVESE |
| | | TASDSTESGDQDEAPRIGGRRAPRRLGGRFFLDMSAESTTGTETDASVSDDPDDT |
| | | SDWSYDDIPPRPKRARVNLRLTSSPDRRDGVIFPKMGRVRSTRETQPRAPTPSAPS |
| | | PNAMLRRSVRQAQRRSSARWTPDLGYMRQCINQLFRVLRVARDPHGSANRLRH |
| | | LIRDCYLMGYCRARLAPRTWCRLLQVSGGTWGMHLRNTIREVEARFDATAEPV |
| | | CKLPCLETRRYGPECDLSNLEIHLSATSDDEISDATDLEAAGSDHTLASQSDTEDA |
| | | PSPVTLETPEPRGSLAVRLEDEFGEFDWTPQEGSQPWLSAVVADTSSVERPGPSD |
| | | SGAGRAAEDRKCLDGCRKMRFSTACPYPCSDTFLRP |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. An engineered cell for AAV production, comprising one or more stably integrated polynucleic acid collectively comprising a nucleic acid sequence encoding for each of: Rep52 or Rep40; Rep78 or Rep68; E2A; E4Orf6; VARNA; VP1; VP2; VP3; and AAP; at least one of which is operably linked to a chemically inducible promoter; wherein:
(i) the nucleic acid sequence encoding for Rep68 lacks the Rep52 start codon;
(ii) the nucleic acid sequence encoding for Rep78 lacks the Rep52 start codon and the Rep68/40 splice site;
(iii) the nucleic acid sequence encoding for Rep52 lacks the Rep40 splice site; and
(iv) the nucleic acid sequence encoding for VP1 lacks start codons for VP2, VP3, and AAP.

2. The engineered cell of claim 1, wherein the engineered cell comprises a chemically inducible promoter comprising the nucleic acid sequence of one or more of SEQ ID NOs: 1-9, operably linked to a nucleic acid sequence encoding for at least one of Rep52, Rep40, Rep78, Rep68, E2A, E4Orf6, VARNA, SC-VP1, VP2, VP3, and AAP.

3. The engineered cell of claim 1 or claim 2, wherein the engineered cell comprises at least two chemically inducible promoters, wherein two or more of the at least two chemically inducible promoters comprise the same nucleic acid sequence.

4. The engineered cell of claim 3, wherein each of the at least two chemically inducible promoters comprise the same nucleic acid sequence.

5. The engineered cell of any one of claims 1-4, wherein the engineered cell comprises a nucleic acid sequence encoding for Rep52 operably linked to a chemically inducible promoter comprising the nucleic acid sequence of one or more of SEQ ID NOs: 1-9, optionally wherein Rep52 comprises the amino acid sequence of SEQ ID NO: 18, optionally wherein the nucleic acid sequence encoding for Rep52 comprises the nucleic acid sequence of SEQ ID NO: 19.

6. The engineered cell of any one of claims 1-5, wherein the engineered cell comprises a nucleic acid sequence encoding for Rep40 operably linked to a chemically inducible promoter comprising the nucleic acid sequence of one or more of SEQ ID NOs: 1-9, optionally wherein Rep40 comprises the amino acid sequence of SEQ ID NO: 20.

7. The engineered cell of any one of claims 1-6, wherein the engineered cell comprises a nucleic acid sequence encoding for Rep78 operably linked to a chemically inducible promoter comprising the nucleic acid sequence of one or more of SEQ ID NOs: 1-9, optionally wherein Rep78 comprises the amino acid sequence of SEQ ID NO: 22, optionally wherein the nucleic acid sequence encoding for Rep78 comprises the nucleic acid sequence of SEQ ID NO: 23.

8. The engineered cell of any one of claims 1-7, wherein the engineered cell comprises a nucleic acid sequence encoding for Rep68 operably linked to a chemically inducible promoter comprising the nucleic acid sequence of one or more of SEQ ID NOs: 1-9, optionally wherein Rep68 comprises the amino acid sequence of SEQ ID NO: 27, optionally wherein the nucleic acid sequence encoding for Rep68 comprises the nucleic acid sequence of SEQ ID NO: 28.

9. The engineered cell of any one of claims 1-8, wherein the engineered cell comprises a nucleic acid sequence encoding for E2A operably linked to a chemically inducible promoter comprising the nucleic acid sequence of one or more of SEQ ID NOs: 1-9, optionally wherein E2A comprises the amino acid sequence of SEQ ID NO: 31.

10. The engineered cell of any one of claims 1-9, wherein the engineered cell comprises a nucleic acid sequence encoding for E4Orf6 operably linked to a chemically inducible promoter comprising the nucleic acid sequence of one or more of SEQ ID NOs: 1-9, optionally wherein E4Orf6 comprises the amino acid sequence of SEQ ID NO: 35, optionally wherein the nucleic acid sequence encoding for E4Orf6 comprises the nucleic acid sequence of SEQ ID NO: 36.

11. The engineered cell of any one of claims 1-10, wherein the engineered cell comprises a nucleic acid sequence encoding for VARNA operably linked to a chemically inducible promoter comprising the nucleic acid sequence of one or more of SEQ ID NOs: 1-9, optionally wherein VARNA comprises the nucleic acid sequence of SEQ ID NO: 38.

12. The engineered cell of any one of claims 1-11, wherein the engineered cell comprises a nucleic acid sequence encoding for VP1 operably linked to a chemically inducible promoter comprising the nucleic acid sequence of one or more of SEQ ID NOs: 1-9, optionally wherein VP1 comprises the amino acid sequence of SEQ ID NO: 31, optionally wherein the nucleic acid sequence encoding for VP1 comprises the nucleic acid sequence of SEQ ID NO: 32.

13. The engineered cell of any one of claims 1-12, wherein the engineered cell comprises a nucleic acid sequence encoding for VP2 operably linked to a chemically inducible promoter comprising the nucleic acid sequence of one or more of SEQ ID NOs: 1-9, optionally wherein VP2 comprises the amino acid sequence of SEQ ID NO: 33.

14. The engineered cell of any one of claims 1-13, wherein the engineered cell comprises a nucleic acid sequence encoding for VP3 operably linked to a chemically inducible promoter comprising the nucleic acid sequence of one or more of SEQ ID NOs: 1-9, optionally wherein VP3 comprises the amino acid sequence of SEQ ID NO: 35.

15. The engineered cell of any one of claims 1-14, wherein the engineered cell comprises a nucleic acid sequence encoding for AAP operably linked to a chemically inducible promoter comprising the nucleic acid sequence of one or more of SEQ ID NOs: 1-9, optionally wherein AAP comprises the amino acid sequence of SEQ ID NO: 37.

16. The engineered cell of any one of claims 1-15, wherein the engineered cell comprises a first stably integrated polynucleic acid, wherein the first stably integrated polynucleic acid comprises the nucleic acid sequence encoding for Rep52 or Rep40 and the nucleic acid sequence encoding for Rep78 or Rep68.

17. The engineered cell of claim 16, wherein the first stably integrated polynucleic acid comprising the nucleic acid sequence encoding for Rep52 and the nucleic acid sequence encoding for Rep78 or Rep68.

18. The engineered cell of claim 17, wherein the nucleic acid sequence encoding for Rep52 and the nucleic acid sequence encoding for Rep78 or Rep68 are each operably linked to a first chemically inducible promoter.

19. The engineered cell of claim 18, wherein the first chemically inducible promoter comprises the nucleic acid sequence of one or more of SEQ ID NOs: 1-9.

20. The engineered cell of any one of claims 16-19, wherein the first stably integrated polynucleic acid further comprises a nucleic acid sequence encoding for an internal ribosome entry site (IRES) or a 2A peptide.

21. The engineered cell of claim 20, wherein the nucleic acid sequence encoding for the IRES or the 2A peptide separates the nucleic acid sequence encoding for Rep52 and the nucleic acid sequence encoding for Rep78 or Rep68.

22. The engineered cell of any one of claims 16-21, wherein the first stably integrated polynucleic acid further comprises a selection marker that is operably linked to a promoter.

23. The engineered cell of any one of claims 1-22, wherein the engineered cell comprises a second stably integrated polynucleic acid, wherein the second stably integrated polynucleic acid comprises the nucleic acid sequence encoding for E2A, the nucleic acid sequence encoding for E4Orf6, and the nucleic acid sequence encoding for VARNA.

24. The engineered cell of claim 23, wherein the nucleic acid sequence encoding for E2A, the nucleic acid sequence encoding for E4Orf6, and the nucleic acid sequence encoding for VARNA are each operably linked to the second chemically inducible promoter.

25. The engineered cell of claim 24, wherein the second chemically inducible promoter comprises the nucleic acid sequence of one or more of SEQ ID NOs: 1-9.

26. The engineered cell of any one of claims 23-25, wherein the second stably integrated polynucleic acid further comprises a nucleic acid sequence encoding for an internal ribosome entry site (IRES) or a 2A peptide.

27. The engineered cell of claim 26, wherein the nucleic acid sequence encoding for the IRES or the 2A peptide separates the nucleic acid sequence encoding for E2A and the nucleic acid sequence encoding E4Orf6.

28. The engineered cell of any one of claims 23-27, wherein the second stably integrated polynucleic acid further comprises a selection marker that is operably linked to a promoter.

29. The engineered cell of any one of claims 1-28, wherein engineered cell comprises a third stably integrated polynucleic acid, wherein the third stably integrated polynucleic acid comprises the nucleic acid sequence encoding for VP1, the nucleic acid sequence encoding for VP2, the nucleic acid sequence encoding for VP3, and the nucleic acid sequence encoding for AAP.

30. The engineered cell of claim 29, wherein the nucleic acid sequence encoding for VP1 is operably linked to a third chemically inducible promoter and the nucleic acid sequence encoding for VP2, the nucleic acid sequence encoding for VP3, and the nucleic acid sequence encoding for AAP are each operably linked to a fourth chemically inducible promoter.

31. The engineered cell of claim 30, wherein the third chemically inducible promoter comprises the nucleic acid sequence of one or more of SEQ ID NOs: 1-9.

32. The engineered cell of claim 30 or claim 31, wherein the fourth chemically inducible promoter comprises the nucleic acid sequence of one or more of SEQ ID NOs: 1-9.

33. The engineered cell of any one of claims 29-32, wherein the third stably integrated polynucleic acid further comprises a selection marker that is operably linked to a promoter.

34. The engineered cell of any one of claims 1-33, wherein the engineered cell comprises a fourth stably integrated polynucleic acid, wherein the fourth stably integrated polynucleic acid comprises a nucleic acid sequence encoding for a transcriptional activator that, when expressed in the presence of a small molecule inducer, binds to a chemically inducible promoter of the engineered cell.

35. The engineered cell of claim 34, wherein the transcriptional activator comprises the amino acid sequence of any one of SEQ ID NOs: 10-16.

36. The engineered cell of claim 34 or claim 35, wherein the fourth stably integrated polynucleic molecule further comprises a selection marker that is operably linked to a promoter.

37. The engineered cell of any one of claims 1-36, wherein the engineered cell further comprises a stable landing pad.

38. The engineered cell of any one of claims 1-37, wherein the engineered cell is derived from a HEK293 cell, a HeLa cell, a BHK cell, or a Sf9 cell.

39. An engineered cell for adeno-associated virus (AAV) production, comprising one or more stably integrated polynucleic acids collectively comprising a nucleic acid sequence encoding for each of: Rep52 or Rep40; Rep78 or Rep68; E2A; E4Orf6; VARNA; VP1; VP2; VP3; and AAP; each of which is operably linked to a chemically inducible promoter.

40. The engineered cell of claim 39, wherein the engineered cell comprises a chemically inducible promoter comprising the nucleic acid sequence of one or more of SEQ ID NOs: 1-9 operably linked to a nucleic acid sequence encoding for at least one of Rep52, Rep40, Rep78, Rep68, E2A, E4Orf6, VARNA, VP1, VP2, VP3, and AAP.

41. The engineered cell of claim 39 or claim 40, wherein the engineered cell comprises at least two chemically inducible promoters, wherein two or more of the at least two chemically inducible promoters comprise the same nucleic acid sequence.

42. The engineered cell of claim 41, wherein each of the at least two chemically inducible promoters comprise the same nucleic acid sequence.

43. The engineered cell of any one of claims 39-42, wherein the engineered cell comprises a nucleic acid sequence encoding for Rep52 operably linked to a chemically inducible promoter comprising the nucleic acid sequence of one or more of SEQ ID NOs: 1-9, optionally wherein Rep52 comprises the amino acid sequence of SEQ ID NO: 18.

44. The engineered cell of any one of claims 39-43, wherein the engineered cell comprises a nucleic acid sequence encoding for Rep40 operably linked to a chemically inducible promoter comprising the nucleic acid sequence of one or more of SEQ ID NOs: 1-9, optionally wherein Rep40 comprises the amino acid sequence of SEQ ID NO: 20.

45. The engineered cell of any one of claims 39-44, wherein the engineered cell comprises a nucleic acid sequence encoding for Rep78 operably linked to a chemically inducible promoter comprising the nucleic acid sequence of one or more of SEQ ID NOs: 1-9, optionally wherein Rep78 comprises the amino acid sequence of any one of SEQ ID NOs: 21 or 22.

46. The engineered cell of any one of claims 39-45, wherein the engineered cell comprises a nucleic acid sequence encoding for Rep68 operably linked to a chemically inducible promoter comprising the nucleic acid sequence of one or more of SEQ ID NOs: 1-9, optionally wherein Rep68 comprises the amino acid sequence of any one of SEQ ID NOs: 26 or 27.

47. The engineered cell of any one of claims 39-46, wherein the engineered cell comprises a nucleic acid sequence encoding for E2A operably linked to a chemically inducible promoter comprising the nucleic acid sequence of one or more of SEQ ID NOs: 1-9, optionally wherein E2A comprises the amino acid sequence of SEQ ID NO: 29.

48. The engineered cell of any one of claims 39-47, wherein the engineered cell comprises a nucleic acid sequence encoding for E4Orf6 operably linked to a chemically inducible promoter comprising the nucleic acid sequence of one or more of SEQ ID NOs: 1-9, optionally wherein E4Orf6 comprises the amino acid sequence of SEQ ID NO: 35.

49. The engineered cell of any one of claims 39-48, wherein the engineered cell comprises a nucleic acid sequence encoding for VARNA operably linked to a chemically inducible promoter comprising the nucleic acid sequence of one or more of SEQ ID NOs: 1-9, optionally wherein VARNA comprises the nucleic acid sequence of SEQ ID NO: 38.

50. The engineered cell of any one of claims 39-49, wherein the engineered cell comprises a nucleic acid sequence encoding for VP1 operably linked to a chemically inducible promoter comprising the nucleic acid sequence of one or more of SEQ ID NOs: 1-9, optionally wherein VP1 comprises the amino acid sequence of any one of SEQ ID NOs: 30 or 31.

51. The engineered cell of any one of claims 39-50, wherein the engineered cell comprises a nucleic acid sequence encoding for VP2 operably linked to a chemically inducible promoter comprising the nucleic acid sequence of one or more of SEQ ID NOs: 1-9, optionally wherein VP2 comprises the amino acid sequence of SEQ ID NO: 33.

52. The engineered cell of any one of claims 39-51, wherein the engineered cell comprises a nucleic acid sequence encoding for VP3 operably linked to a chemically inducible promoter comprising the nucleic acid sequence of one or more of SEQ ID NOs: 1-9, optionally wherein VP3 comprises the amino acid sequence of SEQ ID NO: 34.

53. The engineered cell of any one of claims 39-52, wherein the engineered cell comprises a nucleic acid sequence encoding for AAP operably linked to a chemically inducible promoter comprising the nucleic acid sequence of one or more of SEQ ID NOs: 1-9, optionally wherein AAP comprises the amino acid sequence of SEQ ID NO: 37.

54. The engineered cell of any one of claims 39-53, wherein the engineered cell comprises a first stably integrated polynucleic acid, wherein the first stably integrated polynucleic acid comprises the nucleic acid sequence encoding for Rep52 or Rep40 and the nucleic acid sequence encoding for Rep78 or Rep68.

55. The engineered cell of claim 54, wherein the nucleic acid sequence encoding for Rep52 or Rep40 and the nucleic acid sequence encoding for Rep78 or Rep68 are each operably linked to a first chemically inducible promoter.

56. The engineered cell of claim 55, wherein the first chemically inducible promoter comprises the nucleic acid sequence of one or more of SEQ ID NOs: 1-9.

57. The engineered cell of any one of claims 54-56, wherein the first stably integrated polynucleic acid further comprises a nucleic acid sequence encoding for an internal ribosome entry site (IRES).

58. The engineered cell of claim 57, wherein the nucleic acid sequence encoding for the IRES separates the nucleic acid sequence encoding for Rep52 or Rep40 and the nucleic acid sequence encoding for Rep78 or Rep68.

59. The engineered cell of any one of claims 54-58, wherein the first stably integrated polynucleic acid further comprises a selection marker that is operably linked to a promoter.

60. The engineered cell of any one of claims 39-59, wherein the engineered cell comprises a second stably integrated polynucleic acid, wherein the second stably integrated polynucleic acid comprises the nucleic acid sequences encoding for E2A, E4Orf6, and VARNA.

61. The engineered cell of claim 60, wherein the nucleic acid sequences encoding for E2A, E4Orf6, and VARNA are each operably linked to a second chemically inducible promoter.

62. The engineered cell of claim 61, wherein the second chemically inducible promoter comprises the nucleic acid sequence of one or more of SEQ ID NOs: 1-9.

63. The engineered cell of any one of claims 60-62, wherein the second stably integrated polynucleic acid further comprises a nucleic acid sequence encoding an IRES.

64. The engineered cell of claim 63, wherein the nucleic acid sequence encoding for the IRES separates the nucleic acid sequence encoding for E2A and the nucleic acid sequence encoding for E4Orf6.

65. The engineered cell of any one of claims 60-64, wherein the second stably integrated polynucleic acid further comprises a selection marker that is operably linked to a promoter.

66. The engineered cell of any one of claims 39-65, wherein the engineered cell comprises a third stably integrated polynucleic acid, wherein the third stably integrated polynucleic acid comprises the nucleic acid sequences encoding for VP1, VP2, VP3, and AAP.

67. The engineered cell of claim 66, wherein the third stably integrated polynucleic acid further comprises a selection marker that is operably linked to a promoter.

68. The engineered cell of claim 66 or claim 67, wherein the nucleic acid sequences encoding for VP1, VP2, VP3, and AAP are each operably linked to a third chemically inducible promoter.

69. The engineered cell of claim 68, wherein the third chemically inducible promoter comprises the nucleic acid sequence of one or more of SEQ ID NOs: 1-9.

70. The engineered cell of any one of claims 39-69, further comprising a fourth stably integrated polynucleic acid, wherein the fourth stably integrated polynucleic acid comprises a nucleic acid sequence encoding for a transcriptional activator that, when expressed in the presence of a small molecule inducer, binds to a chemically inducible promoter of the engineered cell.

71. The engineered cell of claim 70, wherein the transcriptional activator comprises the amino acid sequence of any one of SEQ ID NOs: 10-16.

72. The engineered cell of claim 70 or claim 71, wherein the fourth stably integrated polynucleic acid further comprises a selection marker that is operably linked to a promoter.

73. The engineered cell of any one of claims 39-72, wherein the engineered cell further comprises a stable landing pad.

74. The engineered cell of any one of claims 39-73, wherein the engineered cell is derived from a HEK293 cell, a HeLa cell, a BHK cell, or a Sf9 cell.

75. A kit comprising the engineered cell of any one of claims 1-74.

76. The kit of claim 75, further comprising a transfer polynucleic acid molecule comprising, from 5′ to 3′: (i) a nucleic acid sequence of a 5′ AAV inverted tandem repeat (ITR); (ii) a multiple cloning site; and (iii) a nucleic acid sequence of a 3′ AAV inverted tandem repeat (ITR).

77. The kit of claim 76, wherein the transfer polynucleic acid is a plasmid or a vector.

78. The kit of any one of claims 75-77, wherein the kit further comprises a small molecule inducer corresponding to a chemically inducible promoter of the engineered cell.

79. The kit of any one of claims 75-78, wherein the engineered cell comprises a chemically inducible promoter comprising the nucleic acid sequence of one or more of SEQ ID NOs: 1-9 operably linked to the nucleic acid sequence encoding for at least one of Rep52, Rep40, Rep78, Rep68, E2A, E4Orf6, VARNA, VP1, VP2, VP3, and AAP.

80. The kit of any one of claims 75-79, wherein the nucleic acid sequence encoding for Rep52 or Rep40, the nucleic acid sequence encoding for Rep78 or Rep68, the nucleic acid sequence encoding for E2A, the nucleic acid sequence encoding for EOrf6, the nucleic acid sequence encoding for VARNA, the nucleic acid sequence encoding for VP1, the nucleic acid sequence encoding for VP2, the nucleic acid sequence encoding for Vp3, and the nucleic acid sequence encoding for AAP are each operably linked to a chemically inducible promoter comprising the nucleic acid sequence of one or more of SEQ ID NOs: 1-9.

81. The kit of claim 80, wherein the engineered cell comprises at least two chemically inducible promoters.

82. The kit of claim 81, wherein two or more of the at least two chemically inducible promoters are distinct.

83. The kit of any one of claims 75-82, wherein the kit comprise a polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the engineered cell, optionally wherein the engineered cell comprises the polynucleic acid comprising the nucleic acid sequence of the transcriptional activator.

84. The kit of claim 83, wherein the transcriptional activator comprises the amino acid sequence of any one of SEQ ID NOs: 10-12.

85. The kit of claim 83 or claim 84, wherein the kit comprises the small molecule inducer doxycycline or tetracycline.

86. A method of producing an AAV vector comprising:
(a) introducing a transfer polynucleic acid into the engineered cell of any one of claims 1-74; and
(b) contacting the engineered cell with a small molecule inducer corresponding to a chemically inducible promoter of the engineered cell, thereby inducing expression of: Rep52 or Rep40; Rep78 or SC-Rep68; E2A; E4Orf6; VARNA; VP1; VP2; VP3; and AAP;
wherein the engineered cell comprises a heterologous polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the engineered cell;
wherein (b) occurs before, concurrently with, or after (a).

87. The method of claim 86, wherein the engineered cell comprises a chemically inducible promoter comprising the nucleic acid sequence of one or more of SEQ ID NOs: 1-9 operably linked to the nucleic acid sequence encoding for at least one of Rep52, Rep40, Rep78, Rep68, E2A, E4Orf6, VARNA, VP1, VP2, VP3, and AAP.

88. The method of claim 86 or claim 87, wherein the nucleic acid sequence encoding for Rep52 or Rep40, the nucleic acid sequence encoding for Rep78 or Rep68, the nucleic acid sequence encoding for E2A, the nucleic acid sequence encoding for EOrf6, the nucleic acid sequence encoding for VARNA, the nucleic acid sequence encoding for VP1, the nucleic acid sequence encoding for VP2, the nucleic acid sequence encoding for VP3, and the nucleic acid sequence encoding for AAP are each operably linked to a chemically inducible promoter comprising the nucleic acid sequence of one or more of SEQ ID NOs: 1-9.

89. The method of claim 88, wherein the engineered cell comprises at least two chemically inducible promoters.

90. The method of claim 89, wherein two or more of the at least two chemically inducible promoters are distinct.

91. The method of any one of claims 86-90, wherein the transcriptional activator comprises the amino acid sequence of any one of SEQ ID NOs: 10-12.

92. The method of any one of claims 86-91, wherein the small molecule inducer is doxycycline or tetracycline.

93. An engineered cell for AAV production, comprising one or more stably integrated polynucleic acid collectively comprising a nucleic acid sequence encoding for each of: UL5; UL8; UL29; UL30; UL42; and UL52; each of which is operably linked to a chemically inducible promoter.

94. The engineered cell of claim 93, wherein the one or more stably integrated polynucleic acid further comprise a nucleic acid sequence encoding for one or more of UL12, ICP0, ICP4, and ICP22.

95. The engineered cell of claim 94, wherein the one or more stably integrated polynucleic acid further comprise a nucleic acid sequence encoding for each of UL12, ICP0, ICP4, and ICP22.

96. The engineered cell of any one of claims 93-95, wherein the engineered cell comprises a chemically inducible promoter comprising the nucleic acid sequence of one or more of SEQ ID NOs: 1-9 operably linked to a nucleic acid sequence encoding for at least one of UL5, UL8, UL29, UL30, UL42, UL52, UL12, ICP0, ICP4, and ICP22.

97. The engineered cell of any one of claims 93-96, wherein the engineered cell comprises at least two chemically inducible promoters, wherein two or more of the at least two chemically inducible promoters comprise the same nucleic acid sequence.

98. The engineered cell of claim 97, wherein each of the at least two chemically inducible promoters comprise the same nucleic acid sequence.

99. The engineered cell of any one of claims 93-98, wherein the engineered cell comprises a nucleic acid sequence encoding for UL5 operably linked to a chemically inducible promoter comprising the nucleic acid sequence of one or more of SEQ ID NOs: 1-9, optionally wherein UL5 comprises the amino acid sequence of SEQ ID NO: 41.

100. The engineered cell of any one of claims 93-99, wherein the engineered cell comprises a nucleic acid sequence encoding for UL8 operably linked to a chemically inducible promoter comprising the nucleic acid sequence of one or more of SEQ ID NOs: 1-9, optionally wherein UL8 comprises the amino acid sequence of SEQ ID NO: 42.

101. The engineered cell of any one of claims 93-100, wherein the engineered cell comprises a nucleic acid sequence encoding for UL29 operably linked to a chemically inducible promoter comprising the nucleic acid sequence of one or more of SEQ ID NOs: 1-9, optionally wherein UL29 comprises the amino acid sequence of SEQ ID NO: 44.

102. The engineered cell of any one of claims 93-101, wherein the engineered cell comprises a nucleic acid sequence encoding for UL30 operably linked to a chemically inducible promoter comprising the nucleic acid sequence of one or more of SEQ ID NOs: 1-9, optionally wherein UL30 comprises the amino acid sequence of SEQ ID NO: 39.

103. The engineered cell of any one of claims 93-102, wherein the engineered cell comprises a nucleic acid sequence encoding for UL42 operably linked to a chemically inducible promoter comprising the nucleic acid sequence of one or more of SEQ ID NOs: 1-9, optionally wherein UL42 comprises the amino acid sequence of SEQ ID NO: 40.

104. The engineered cell of any one of claims 93-103, wherein the engineered cell comprises a nucleic acid sequence encoding for UL52 operably linked to a chemically inducible promoter comprising the nucleic acid sequence of one or more of SEQ ID NOs: 1-9, optionally wherein UL52 comprises the amino acid sequence of SEQ ID NO: 43.

105. The engineered cell of any one of claims 94-104, wherein the engineered cell comprises a nucleic acid sequence encoding for UL12 operably linked to a chemically inducible promoter comprising the nucleic acid sequence of one or more of SEQ ID NOs: 1-9, optionally wherein UL12 comprises the amino acid sequence of SEQ ID NO: 50.

106. The engineered cell of any one of claims 94-105, wherein the engineered cell comprises a nucleic acid sequence encoding for ICP0 operably linked to a chemically inducible promoter comprising the nucleic acid sequence of one or more of SEQ ID NOs: 1-9, optionally wherein ICP0 comprises the amino acid sequence of SEQ ID NO: 51.

107. The engineered cell of any one of claims 94-106, wherein the engineered cell comprises a nucleic acid sequence encoding for ICP4 operably linked to a chemically inducible promoter comprising the nucleic acid sequence of one or more of SEQ ID NOs: 1-9, optionally wherein ICP4 comprises the amino acid sequence of SEQ ID NO: 52.

108. The engineered cell of any one of claims 94-107, wherein the engineered cell comprises a nucleic acid sequence encoding for ICP22 operably linked to a chemically inducible promoter comprising the nucleic acid sequence of one or more of SEQ ID NOs: 1-9, optionally wherein ICP22 comprises the amino acid sequence of SEQ ID NO: 53.

109. The engineered cell of any one of claims 93-108, wherein the engineered cell comprises a first stably integrated polynucleic acid, wherein the first stably integrated polynucleic acid comprises the nucleic acid sequence encoding for UL30 and the nucleic acid sequence encoding for UL42.

110. The engineered cell of claim 109, wherein the nucleic acid sequence encoding for UL30 and the nucleic acid sequence encoding for UL42 are each operably linked to a first chemically inducible promoter.

111. The engineered cell of claim 110, wherein the first chemically inducible promoter comprises the nucleic acid sequence of one or more of SEQ ID NOs: 1-9.

112. The engineered cell of any one of claims 109-111, wherein the first stably integrated polynucleic acid further comprises a nucleic acid sequence encoding for an internal ribosome entry site (IRES).

113. The engineered cell of claim 112, wherein the nucleic acid sequence encoding for the IRES separates the nucleic acid sequence encoding for UL30 and the nucleic acid sequence encoding for UL42.

114. The engineered cell of any one of claims 109-113, wherein the first stably integrated polynucleic acid further comprises a selection marker that is operably linked to a promoter.

115. The engineered cell of any one of claims 93-114, the engineered cell comprises a second stably integrated polynucleic acid, wherein the second stably integrated polynucleic acid comprises the nucleic acid sequence encoding for UL5, the nucleic acid sequence encoding for UL8, the nucleic acid sequence encoding for UL52, and the nucleic acid sequence encoding for UL29.

116. The engineered cell of claim 115, wherein the nucleic acid sequence encoding for UL5, the nucleic acid sequence encoding for UL8, and the nucleic acid sequence encoding for UL52 are each operably linked to a second chemically inducible promoter, and the nucleic acid sequence encoding for UL29 is operably linked to a third chemically inducible promoter.

117. The engineered cell of claim 116, wherein the second chemically inducible promoter comprises the nucleic acid sequence of one or more of SEQ ID NOs: 1-9.

118. The engineered cell of claim 116 or claim 117, wherein the third chemically inducible promoter the nucleic acid sequence of one or more of SEQ ID NOs: 1-9.

119. The engineered cell of any one of claims 115-118, wherein the second stably integrated polynucleic acid further comprises a nucleic acid sequence encoding for an internal ribosome entry site (IRES).

120. The engineered cell of claim 119, wherein the nucleic acid sequence encoding for the IRES separates the nucleic acid sequence encoding for UL8 and the nucleic acid sequence encoding UL52.

121. The engineered cell of any one of claims 115-120, wherein the second stably integrated polynucleic acid further comprises a selection marker that is operably linked to a promoter.

122. The engineered cell of any one of claims 93-121, wherein the engineered cell comprises a third stably integrated polynucleic acid, wherein the third stably integrated polynucleic acid comprises a nucleic acid sequence encoding for a transcriptional activator that, when expressed in the presence of a small molecule inducer, binds to a chemically inducible promoter of the engineered cell.

123. The engineered cell of claim 122, wherein the transcriptional activator comprises the amino acid sequence of any one of SEQ ID NOs: 10-16.

124. The engineered cell of claim 122 or claim 123, wherein the third stably integrated polynucleic acid further comprises a selection marker that is operably linked to a promoter.

125. The engineered cell of any one of claims 93-124, wherein the engineered cell comprises a fourth stably integrated polynucleic acid, wherein the fourth stably integrated polynucleic acid comprises the nucleic acid sequence encoding for ICP0, the nucleic acid sequence encoding for UL12, the nucleic acid sequence encoding for ICP4, and the nucleic acid sequence encoding for ICP22.

126. The engineered cell of claim 125, wherein the nucleic acid sequence encoding for ICP0, the nucleic acid sequence encoding for UL12, and the nucleic acid sequence encoding for ICP4, are each linked to a fourth chemically inducible promoter and the nucleic acid sequence encoding for ICP22 is operably linked to a fifth chemically inducible promoter.

127. The engineered cell of claim 126, wherein the fourth chemically inducible promoter and fifth chemically inducible promoter comprises the nucleic acid sequence of one or more of SEQ ID NOs: 1-9.

128. The engineered cell of any one of claims 125-127, wherein the first stably integrated polynucleic acid further comprises a nucleic acid sequence encoding for an internal ribosome entry site (IRES).

129. The engineered cell of claim 128, wherein the nucleic acid sequence encoding for the IRES separates the nucleic acid sequence encoding for UL12 and the nucleic acid sequence encoding for ICP4.

130. The engineered cell of any one of claims 125-128, wherein the first stably integrated polynucleic acid further comprises a selection marker that is operably linked to a promoter.

131. The engineered cell of any one of claims 93-130, wherein the engineered cell further comprises a stable landing pad.

132. The engineered cell of any one of claims 93-131, wherein the engineered cell is derived from a HEK293 cell, a HeLa cell, a BHK cell, or a Sf9 cell.

133. A kit comprising the engineered cell of any one of claims 93-132.

134. The kit of claim 133, further comprising a transfer polynucleic acid molecule comprising, from 5′ to 3′: (i) a nucleic acid sequence of a 5′ AAV inverted tandem repeat (ITR); (ii) a multiple cloning site; and (iii) a nucleic acid sequence of a 3′ AAV inverted tandem repeat (ITR).

135. The kit of claim 134, wherein the transfer polynucleic acid is a plasmid or a vector.

136. The kit of any one of claims 133-135, wherein the kit further comprises a small molecule inducer corresponding to a chemically inducible promoter of the engineered cell.

137. The kit of any one of claims 133-136, wherein the engineered cell comprises a chemically inducible promoter comprising the nucleic acid sequence of one or more of SEQ ID NOs: 1-9 operably linked to the nucleic acid sequence encoding for at least one of ICP0, ICP4, ICP22, UL5, UL8, UL12, UL29, UL30, UL42, and UL52.

138. The kit of any one of claims 133-137, wherein the nucleic acid sequence encoding for ICP0, the nucleic acid sequence encoding for ICP4, the nucleic acid sequence encoding for ICP22, the nucleic acid sequence encoding for UL5, the nucleic acid sequence encoding for UL8, the nucleic acid sequence encoding for UL12, the nucleic acid sequence encoding for UL29, the nucleic acid sequence encoding for UL30, the nucleic acid sequence encoding for UL42, and the nucleic acid sequence encoding for UL52 are each operably linked to a chemically inducible promoter comprising the nucleic acid sequence of one or more of SEQ ID NOs: 1-9.

139. The kit of claim 138, wherein the engineered cell comprises at least two chemically inducible promoters.

140. The kit of claim 139, wherein two or more of the at least two chemically inducible promoters are distinct.

141. The kit of any one of claims 133-139, wherein the kit comprise a polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the engineered cell, optionally wherein the engineered cell comprises the polynucleic acid comprising the nucleic acid sequence of the transcriptional activator.

142. The kit of claim 141, wherein the transcriptional activator comprises the amino acid sequence of any one of SEQ ID NOs: 10-12.

143. The kit of claim 141 or claim 142, wherein the kit comprises the small molecule inducer doxycycline or tetracycline.

144. A method of producing an AAV vector comprising:
(a) introducing a transfer polynucleic acid into the engineered cell of any one of claims 93-132; and
(b) contacting the engineered cell with a small molecule inducer corresponding to a chemically inducible promoter of the engineered cell, thereby inducing expression of: UL5, UL8, UL29, UL30, UL42, and UL52;
wherein the engineered cell comprises a heterologous polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the engineered cell;
wherein (b) occurs before, concurrently with, or after (a).

145. The method of claim 144, wherein the engineered cell comprises the engineered cell comprises one or more polynucleic acids collectively comprising a nucleic acid sequence encoding for each of: Rep52 or Rep40; Rep78 or Rep68; E2A; E4Orf6; VARNA; VP1; VP2; VP3; and AAP.

146. The method of claim 144 or claim 145, wherein the engineered cell comprise one or more stably integrated polynucleic acids collectively comprising a nucleic acid sequence encoding for each of: Rep52 or Rep40; Rep78 or Rep68; E2A; E4Orf6; VARNA; VP1; VP2; VP3; and AAP.

147. The method of claim 144 or claim 145, wherein the method further comprises: (c) introducing one or more polynucleic acids collectively comprising a nucleic acid sequence encoding for each of: Rep52 or Rep40; Rep78 or Rep68; E2A; E4Orf6; VARNA; VP1; VP2; VP3; and AAP; wherein (c) occurs prior to, concurrently with, or after (a) or (b).

148. The method of any one of claims 144-147, wherein the engineered cell comprises a chemically inducible promoter comprising the nucleic acid sequence of one or more of SEQ ID NOs: 1-9 operably linked to the nucleic acid sequence encoding for at least one of ICP0, ICP4, ICP22, UL5, UL8, UL12, UL29, UL30, UL42, and UL52.

149. The method of any one of claims 144-148, wherein the nucleic acid sequence encoding for Rep52 or Rep40, the nucleic acid sequence encoding for ICP0, the nucleic acid sequence encoding for ICP4, the nucleic acid sequence encoding for ICP22, the nucleic acid sequence encoding for UL5, the nucleic acid sequence encoding for UL8, the nucleic acid sequence encoding for UL12, the nucleic acid sequence encoding for UL29, the nucleic acid sequence encoding for UL30, the nucleic acid sequence encoding for UL42, and the nucleic acid sequence encoding for UL52 are each operably linked to a chemically inducible promoter comprising the nucleic acid sequence of one or more of SEQ ID NOs: 1-9.

150. The method of claim 149, wherein the engineered cell comprises at least two chemically inducible promoters.

151. The method of claim 150, wherein two or more of the at least two chemically inducible promoters are distinct.

152. The method of any one of claims 144-151, wherein the transcriptional activator comprises the amino acid sequence of any one of SEQ ID NOs: 10-12.

153. The method of any one of claims 144-152, wherein the small molecule inducer is doxycycline or tetracycline.


SEQUENCE LISTING (53 sequences)

SEQ ID NO: 1 - DNA, 346 bp, synthetic construct
  source 1..346: mol_type = other DNA
ctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggagaattcgagctcggtacccgggga

SEQ ID NO: 2 - DNA, 423 bp, synthetic construct
  source 1..423: mol_type = other DNA
gtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctatcgatcggccggataacgccacc

SEQ ID NO: 3 - DNA, 496 bp, synthetic construct
  source 1..496: mol_type = other DNA
gaattctccaggcgatctgacggttcactaaacgagctctgcttatataggcctcccaccgtacacgccacctcgacatactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctccgcggggatcc

SEQ ID NO: 4 - DNA, 12 bp, synthetic construct
  source 1..12: mol_type = other DNA
attggatccaat

SEQ ID NO: 5 - DNA, 28 bp, synthetic construct
  source 1..28: mol_type = other DNA
tatttacaaacaaccatgaatgtaagta

SEQ ID NO: 6 - DNA, 18 bp, synthetic construct
  source 1..18: mol_type = other DNA
cggagtactgtcctccga

SEQ ID NO: 7 - DNA, 30 bp, synthetic construct
  source 1..30: mol_type = other DNA
atgatacgaaacgtaccgtatcgttaaggt

SEQ ID NO: 8 - DNA, 26 bp, synthetic construct
  source 1..26: mol_type = other DNA
agaaacaaaccaacctgtctgtatta

SEQ ID NO: 9 - DNA, 28 bp, synthetic construct
  source 1..28: mol_type = other DNA
aacaaacagacaatctggtctgtttgta

SEQ ID NO: 10 - protein, 249 aa, synthetic construct
MSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYELTLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 11 - protein, 248 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 12 - protein, 298 aa, synthetic construct
MDMPRIKPGQRVMMALRKMIASGEIKSGERIAEIPTAAALGVSRMPVRIALRSLEQEGLVVRLGARGYAARGVSSDQIRDAIEVRGVLEGFAARRLAERGMTAETHARFVVLIAEGEALFAAGRLNGEDLDRYAAYNQAFHDTLVSAAGNGAVESALARNGFEPFAAAGALALDLMDLSAEYEHLLAAHRQHQAVLDAVSCGDAEGAERIMRDHALAAIRNAKVFEAAASAGAPLGAAWSIRADSGGGGPTDALDDFDLDMLPADALDDFDLDMLPADALDDFDLDMLPGPPKKKRKV

SEQ ID NO: 13 - protein, 264 aa, synthetic construct
MVRRTKEEAQETRAQIIEAAERAFYKRGVARTTLADIAELAGVTRGAIYWHFNNKAELVQALLDSLHETHDHLARASESEDEVDPLGCMRKLLLQVFNELVLDARTRRINEILHHKCEFTDDMCEIRQQHQSAVLDCHKGITLTLANVVRRGQLPGELDAERAAVAMFAYVDGLIRRWLLLPDSVDLLGDVEKWVDTGLDMLRLSPALRKSGGGGPTDALDDFDLDMLPADALDDFDLDMLPADALDDFDLDMLPGPPKKKRKV

SEQ ID NO: 14 - protein, 254 aa, synthetic construct
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQCSGGGGPTDALDDFDLDMLPADALDDFDLDMLPADALDDFDLDMLPGPPKKKRKV

SEQ ID NO: 15 - protein, 257 aa, synthetic construct
MSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSIGLDLIVAADRDPALREGIQRTVERNRFVVEDMWLGVLVSRGLSRDDAEDILWLIFNSVRGLVVRSLWQKDKERFERVRNSTLEIARERYAKFKRSGGGGPTDALDDFDLDMLPADALDDFDLDMLPADALDDFDLDMLPGPPKKKRKV

SEQ ID NO: 16 - protein, 260 aa, synthetic construct
MVIMSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSIGLDLIVAADRDPVLREGIQRTVERNRFVVGDIWLGVLVSRGLSRDDAEDILWLIFNSVRGLVVRSLWQKDKERFERVRNSTLEIARERYAKFKRSGGGGPTDALDDFDLDMLPADALDDFDLDMLPADALDDFDLDMLPGPPKKKRKV

SEQ ID NO: 17 - DNA, 1932 bp, unidentified
  source 1..1932: mol_type = genomic DNA; note = Adeno-associated virus 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 18 - protein, 397 aa, synthetic construct
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 19 - DNA, 1194 bp, synthetic construct
  source 1..1194: mol_type = other DNA
atggaattagtgggctggttggtcgataaaggcatcacaagcgaaaaacaatggattcaagaagatcaagcgagctatattagttttaacgccgctagtaatagcagaagtcagattaaagccgctctcgataacgccggcaaaatcatgtctttaaccaagacagctcctgattatttagtcgggcaacaacctgtcgaggacatcagttctaacagaatctacaagatcctcgaattgaatggctatgaccctcagtacgccgccagtgtgttcttaggctgggctaccaagaaatttgggaaacgcaatacaatttggttattcggccccgccaccacaggcaaaacaaatattgccgaagctatcgctcataccgtccctttctatggctgtgtgaattggacaaacgaaaatttcccttttaatgattgcgtggataaaatggtcatttggtgggaagaaggcaaaatgacagctaaagtggtcgaaagcgctaaggctatcttgggcggctctaaagtcagagtcgatcaaaagtgtaaaagtagcgctcaaatcgatcccacccctgtcattgtgacaagtaatacaaatatgtgtgctgtcatcgatggcaatagcaccacatttgagcatcaacaacccctccaggatagaatgtttaagttcgagttgacaagaagattagaccacgatttcggcaaagtgacaaaacaagaggtgaaggatttctttagatgggccaaagaccatgtcgtggaagtcgaacacgagttttatgtgaagaaaggcggcgctaaaaagcggcctgctccttccgatgccgacatctccgaacctaagagagtcagagaaagcgtggcccaacccagcaccagcgatgccgaggccagcattaattatgccgatcgctatcagaataagtgcagcagacatgtcgggatgaacttaatgttattcccttgtcggcagtgtgaacggatgaaccaaaacagcaacatttgttttacccacggacaaaaggattgcctggaatgtttccctgtcagcgagagccagcctgtgagcgtggtgaagaaagcctaccaaaagttatgttatatccaccacattatgggcaaagtccccgatgcctgtaccgcttgtgacttagtgaacgtagacctcgacgattgtattttcgagcagtga

SEQ ID NO: 20 - protein, 312 aa, synthetic construct
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 21 - protein, 621 aa, synthetic construct
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 22 - protein, 621 aa, synthetic construct
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYGELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 23 - DNA, 1866 bp, synthetic construct
  source 1..1866: mol_type = other DNA
atgcctggcttctacgagatcgtgatcaaggtgcccagcgacctggacgagcatctgcctggcatcagcgacagcttcgtgaattgggtcgccgagaaagagtgggagctgcctcctgacagcgacatggacctgaacctgattgagcaggcccctctgacagtggccgagaagctgcagagggatttcctgacagagtggcggagagtgtctaaggcccctgaggctctgttcttcgtgcagttcgagaagggcgagagctacttccacatgcacgtgctggtcgagacaaccggcgtgaagtctatggtgctgggcagattcctgagccagatcagagagaagctgatccagcggatctaccggggcatcgagcccacactgcctaattggttcgccgtgaccaagaccagaaacggtgctggcggcggaaacaaggtggtggacgagtgctacatccccaactacctgctgcctaagacacagcccgaactgcagtgggcctggaccaacatggaacagtacctgagcgcctgcctgaatctgaccgagcggaaaagactggtggcccagcatctgacccacgtgtcccagacacaagagcagaacaaagagaatcagaaccccaacagcgacgcccctgtgatcagaagcaagaccagcgccagatacggagaactcgttggctggctggtggacaagggcatcacaagcgagaagcagtggatccaagaggaccaggccagctacatcagcttcaacgccgcctccaacagcagatcccagatcaaggccgctctggacaacgccggcaagatcatgagcctgacaaagacagcccctgactacctcgtgggccagcagcctgtggaagatatcagcagcaaccggatctacaagatcctggaactgaacggctacgaccctcagtatgccgcctctgtgtttctcggctgggctaccaagaagttcggcaagcggaacaccatctggctgtttggccctgccacaaccggcaagaccaatatcgccgaggctatcgcccacaccgtgcctttttacggctgcgtgaactggaccaatgagaacttccccttcaacgactgcgtggacaagatggtcatttggtgggaagagggcaagatgaccgccaaagtggtggaaagcgccaaggccatcctcggcggatctaaagttcgcgtggaccagaagtgcaagtctagcgcccagatcgaccccacacctgtgatcgtgaccagcaacaccaacatgtgcgccgtgatcgacggcaacagcaccacctttgaacaccagcagccactgcaggaccggatgttcaagttcgagctgaccagacggctggaccacgacttcggcaaagtgaccaagcaagaagtgaaggacttcttccgctgggccaaagatcacgtggtggaagtggaacacgagttctacgtgaagaaaggcggagccaagaagaggcccgctccttccgatgccgatatcagcgagcctaagcgcgtgcgggaatctgtggctcagcctagcacatctgatgccgaggccagcatcaactacgccgacagataccagaacaagtgcagccggcacgtgggaatgaatctgatgctgttcccctgtcggcagtgcgagcggatgaaccagaacagcaacatctgcttcacccacggccagaaagactgcctggaatgcttccccgtgtccgagtctcagcctgtgtccgtggtcaagaaggcctaccagaagctgtgttacatccaccacatcatgggcaaagtgcccgatgcctgcaccgcctgcgatctggttaatgtggacctggatgactgcatcttcgagcagtga

SEQ ID NO: 24 - DNA, 1866 bp, synthetic construct
  source 1..1866: mol_type = other DNA
ctggcgggcttctacgagatcgtgatcaaggtgcccagcgacctggacgagcatctgcctggcatcagcgacagcttcgtgaattgggtcgccgagaaagagtgggagctgcctcctgacagcgacttggacctgaacctgattgagcaggcccctctgacagtggccgagaagctgcagagggatttcctgacagagtggcggagagtgtctaaggcccctgaggctctgttcttcgtgcagttcgagaagggcgagagctacttccacttacacgtgctggtcgagacaaccggcgtgaagtctttagtgctgggcagattcctgagccagatcagagagaagctgatccagcggatctaccggggcatcgagcccacactgcctaattggttcgccgtgaccaagaccagaaacggcgctggcggcggaaacaaggtggtggacgagtgctacatccccaactacctgctgcctaagacacagcccgaactgcagtgggcctggaccaacttagaacagtacctgagcgcctgcctgaatctgaccgagcggaaaagactggtggcccagcatctgacccacgtgtcccagacacaagagcagaacaaagagaatcagaaccccaacagcgacgcccctgtgatcagaagcaagaccagcgccagatacatggaactcgttggctggctggtggacaagggcatcacaagcgagaagcagtggatccaagaggaccaggccagctacatcagcttcaacgccgcctccaacagcagatcccagatcaaggccgctctggacaacgccggcaagatcatgagcctgacaaagacagcccctgactacctcgtgggccagcagcctgtggaagatatcagcagcaaccggatctacaagatcctggaactgaacggctacgaccctcagtatgccgcctctgtgtttctcggctgggctaccaagaagttcggcaagcggaacaccatctggctgtttggccctgccacaaccggcaagaccaatatcgccgaggctatcgcccacaccgtgcctttttacggctgcgtgaactggaccaatgagaacttccccttcaacgactgcgtggacaagatggtcatttggtgggaagagggcaagatgaccgccaaagtggtggaaagcgccaaggccatcctcggcggatctaaagttcgcgtggaccagaagtgcaagtctagcgcccagatcgaccccacacctgtgatcgtgaccagcaacaccaacatgtgcgccgtgatcgacggcaacagcaccacctttgaacaccagcagccactgcaggaccggatgttcaagttcgagctgaccagacggctggaccacgacttcggcaaagtgaccaagcaagaagtgaaggacttcttccgctgggccaaagatcacgtggtggaagtggaacacgagttctacgtgaagaaaggcggagccaagaagaggcccgctccttccgatgccgatatcagcgagcctaagcgcgtgcgggaatctgtggctcagcctagcacatctgatgccgaggccagcatcaactacgccgacagataccagaacaagtgcagccggcacgtgggaatgaatctgatgctgttcccctgtcggcagtgcgagcggatgaaccagaacagcaacatctgcttcacccacggccagaaagactgcctggaatgcttccccgtgtccgagtctcagcctgtgtccgtggtcaagaaggcctaccagaagctgtgttacatccaccacatcatgggcaaagtgcccgatgcctgcaccgcctgcgatctggttaatgtggacctggatgactgcatcttcgagcagtga

SEQ ID NO: 25 - DNA, 3648 bp, synthetic construct
  source 1..3648: mol_type = other DNA
gccgccaccatggaattagtgggctggttggtcgataaaggcatcacaagcgaaaaacaatggattcaagaagatcaagcgagctatattagttttaacgccgctagtaatagcagaagtcagattaaagccgctctcgataacgccggcaaaatcatgtctttaaccaagacagctcctgattatttagtcgggcaacaacctgtcgaggacatcagttctaacagaatctacaagatcctcgaattgaatggctatgaccctcagtacgccgccagtgtgttcttaggctgggctaccaagaaatttgggaaacgcaatacaatttggttattcggccccgccaccacaggcaaaacaaatattgccgaagctatcgctcataccgtccctttctatggctgtgtgaattggacaaacgaaaatttcccttttaatgattgcgtggataaaatggtcatttggtgggaagaaggcaaaatgacagctaaagtggtcgaaagcgctaaggctatcttgggcggctctaaagtcagagtcgatcaaaagtgtaaaagtagcgctcaaatcgatcccacccctgtcattgtgacaagtaatacaaatatgtgtgctgtcatcgatggcaatagcaccacatttgagcatcaacaacccctccaggatagaatgtttaagttcgagttgacaagaagattagaccacgatttcggcaaagtgacaaaacaagaggtgaaggatttctttagatgggccaaagaccatgtcgtggaagtcgaacacgagttttatgtgaagaaaggcggcgctaaaaagcggcctgctccttccgatgccgacatctccgaacctaagagagtcagagaaagcgtggcccaacccagcaccagcgatgccgaggccagcattaattatgccgatcgctatcagaataagtgcagcagacatgtcgggatgaacttaatgttattcccttgtcggcagtgtgaacggatgaaccaaaacagcaacatttgttttacccacggacaaaaggattgcctggaatgtttccctgtcagcgagagccagcctgtgagcgtggtgaagaaagcctaccaaaagttatgttatatccaccacattatgggcaaagtccccgatgcctgtaccgcttgtgacttagtgaacgtagacctcgacgattgtattttcgagcagtgataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgcctggcttctacgagatcgtgatcaaggtgcccagcgacctggacgagcatctgcctggcatcagcgacagcttcgtgaattgggtcgccgagaaagagtgggagctgcctcctgacagcgacatggacctgaacctgattgagcaggcccctctgacagtggccgagaagctgcagagggatttcctgacagagtggcggagagtgtctaaggcccctgaggctctgttcttcgtgcagttcgagaagggcgagagctacttccacatgcacgtgctggtcgagacaaccggcgtgaagtctatggtgctgggcagattcctgagccagatcagagagaagctgatccagcggatctaccggggcatcgagcccacactgcctaattggttcgccgtgaccaagaccagaaacggtgctggcggcggaaacaaggtggtggacgagtgctacatccccaactacctgctgcctaagacacagcccgaactgcagtgggcctggaccaacatggaacagtacctgagcgcctgcctgaatctgaccgagcggaaaagactggtggcccagcatctgacccacgtgtcccagacacaagagcagaacaaagagaatcagaaccccaacagcgacgcccctgtgatcagaagcaagaccagcgccagatacggagaactcgttggctggctggtggacaagggcatcacaagcgagaagcagtggatccaagaggaccaggccagctacatcagcttcaacgccgcctccaacagcagatcccagatcaaggccgctctggacaacgccggcaagatcatgagcctgacaaagacagcccctgactacctcgtgggccagcagcctgtggaagatatcagcagcaaccggatctacaagatcctggaactgaacggctacgaccctcagtatgccgcctctgtgtttctcggctgggctaccaagaagttcggcaagcggaacaccatctggctgtttggccctgccacaaccggcaagaccaatatcgccgaggctatcgcccacaccgtgcctttttacggctgcgtgaactggaccaatgagaacttccccttcaacgactgcgtggacaagatggtcatttggtgggaagagggcaagatgaccgccaaagtggtggaaagcgccaaggccatcctcggcggatctaaagttcgcgtggaccagaagtgcaagtctagcgcccagatcgaccccacacctgtgatcgtgaccagcaacaccaacatgtgcgccgtgatcgacggcaacagcaccacctttgaacaccagcagccactgcaggaccggatgttcaagttcgagctgaccagacggctggaccacgacttcggcaaagtgaccaagcaagaagtgaaggacttcttccgctgggccaaagatcacgtggtggaagtggaacacgagttctacgtgaagaaaggcggagccaagaagaggcccgctccttccgatgccgatatcagcgagcctaagcgcgtgcgggaatctgtggctcagcctagcacatctgatgccgaggccagcatcaactacgccgacagataccagaacaagtgcagccggcacgtgggaatgaatctgatgctgttcccctgtcggcagtgcgagcggatgaaccagaacagcaacatctgcttcacccacggccagaaagactgcctggaatgcttccccgtgtccgagtctcagcctgtgtccgtggtcaagaaggcctaccagaagctgtgttacatccaccacatcatgggcaaagtgcccgatgcctgcaccgcctgcgatctggttaatgtggacctggatgactgcatcttcgagcagtga

SEQ ID NO: 26 - protein, 536 aa, synthetic construct
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 27 - protein, 536 aa, synthetic construct
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYGELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 28 - DNA, 1611 bp, synthetic construct
  source 1..1611: mol_type = other DNA
atgcctggcttctacgagatcgtgatcaaggtgcccagcgacctggacgagcatctgcctggcatcagcgacagcttcgtgaattgggtcgccgagaaagagtgggagctgcctcctgacagcgacatggacctgaacctgattgagcaggcccctctgacagtggccgagaagctgcagagggatttcctgacagagtggcggagagtgtctaaggcccctgaggctctgttcttcgtgcagttcgagaagggcgagagctacttccacatgcacgtgctggtcgagacaaccggcgtgaagtctatggtgctgggcagattcctgagccagatcagagagaagctgatccagcggatctaccggggcatcgagcccacactgcctaattggttcgccgtgaccaagaccagaaacggtgctggcggcggaaacaaggtggtggacgagtgctacatccccaactacctgctgcctaagacacagcccgaactgcagtgggcctggaccaacatggaacagtacctgagcgcctgcctgaatctgaccgagcggaaaagactggtggcccagcatctgacccacgtgtcccagacacaagagcagaacaaagagaatcagaaccccaacagcgacgcccctgtgatcagaagcaagaccagcgccagatacggagaactcgttggctggctggtggacaagggcatcacaagcgagaagcagtggatccaagaggaccaggccagctacatcagcttcaacgccgcctccaacagcagatcccagatcaaggccgctctggacaacgccggcaagatcatgagcctgacaaagacagcccctgactacctcgtgggccagcagcctgtggaagatatcagcagcaaccggatctacaagatcctggaactgaacggctacgaccctcagtatgccgcctctgtgtttctcggctgggctaccaagaagttcggcaagcggaacaccatctggctgtttggccctgccacaaccggcaagaccaatatcgccgaggctatcgcccacaccgtgcctttttacggctgcgtgaactggaccaatgagaacttccccttcaacgactgcgtggacaagatggtcatttggtgggaagagggcaagatgaccgccaaagtggtggaaagcgccaaggccatcctcggcggatctaaagttcgcgtggaccagaagtgcaagtctagcgcccagatcgaccccacacctgtgatcgtgaccagcaacaccaacatgtgcgccgtgatcgacggcaacagcaccacctttgaacaccagcagccactgcaggaccggatgttcaagttcgagctgaccagacggctggaccacgacttcggcaaagtgaccaagcaagaagtgaaggacttcttccgctgggccaaagatcacgtggtggaagtggaacacgagttctacgtgaagaaaggcggagccaagaagaggcccgctccttccgatgccgatatcagcgagcctaagcgcgtgcgggaatctgtggctcagcctagcacatctgatgccgaggccagcatcaactacgccgacagactggccaggggccacagcctgtga

SEQ ID NO: 29 - protein, 529 aa, synthetic construct
MASREEEQRETTPERGRGAARRPPTMEDVSSPSPSPPPPRAPPKKRLRRRLESEDEEDSSQDALVPRTPSPRPSTSTADLAIASKKKKKRPSPKPERPPSPEVIVDSEEEREDVALQMVGFSNPPVLIKHGKGGKRTVRRLNEDDPVARGMRTQEEKEESSEAESESTVINPLSLPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 30 - protein, 735 aa, synthetic construct [sequence omitted]

SEQ ID NO: 31 - protein, 735 aa, synthetic construct [sequence omitted]

SEQ ID NO: 32 - DNA, 2208 bp, synthetic construct
  source 1..2208: mol_type = other DNA
atggccgctgacggctacttgcccgactggttggaagataccttgagcgagggcatccggcaatggtggaaattgaagcccggacctccacctcccaaaccagccgaaagacacaaagatgattctcgcggcttggtcttgcccggctataaatatttgggcccttttaatggcttggataaaggcgaacccgtgaatgaagccgatgctgccgctttggaacatgataaggcttatgatagacaattggattctggcgataatccctatttgaaatataatcatgctgatgccgaattccaagaaagattgaaggaggacaccagcttcggcgggaatttgggcagggccgtgtttcaagccaagaaacgcgtgttggagcccttagggttagtggaagagcccgtgaaaaccgcccctggcaagaaacgccccgtggaacatagccccgtcgaacccgatagtagtagcggcacaggcaaagccgggcaacaacccgcccggaagcggctcaacttcggccaaaccggcgatgccgatagcgtgcccgatcctcaacccttgggccaaccacccgccgctcctagcggcttaggcaccaacactatggccaccgggtccggtgcccctatggccgataacaatgaaggggctgatggcgtcggcaacagtagcggcaactggcactgtgacagtacctggatgggtgatcgggtgattacaacatctacaaggacatgggctttacctacatataataatcatttgtataagcagatcagttctcagagcggcgcaagcaatgataaccattatttcgggtattctacaccctggggctacttcgattttaatcggtttcattgtcatttcagccccagagattggcagcggttgattaataataattggggctttaggcctaaacggttgaattttaaattgttcaatatccaggtgaaggaagtgacccaaaacgatggcaccaccaccatcgctaacaatttgacatctaccgtgcaagtcttcaccgatagcgaatatcaattgccctatgtgttggggagcgcccaccagggctgtttgcctccctttcccgccgatgtgtttatggtcccgcaatacggctatttgacattaaataatggctcccaagccgtgggcagaagcagcttctattgtttagaatatttccccagccaaatgttaagaacaggcaataatttcacattctcttataccttcgaagatgtgccctttcattcttcttatgcccattctcaatccttagatagattgatgaaccccttgattgatcaatatttatactatctctctcggaccaataccccctccggcacaacaacccaaagccgcttgcaattcagccaagctggcgcctccgatatcagagatcaaagccgcaattggttgcccggcccttgctatagacaacaaagggtgagcaaaaccagcgccgacaataataattccgagtatagctggaccggcgccacaaaatatcatttgaacgggcgggatagcttagtcaaccccgggcccgctatggcctctcataaagatgacgaggagaaattcttcccacaatctggcgtgttgattttcggcaaacaggggagcgaaaagaccaacgtcgatattgagaaagtgatgatcaccgatgaggaagagattcgcaccacaaaccctgtcgccaccgaacaatacggcagcgtgagcacaaatttgcaacggggtaatcggcaggccgccacagccgacgtgaatacccagggagtgttgcccgggatggtgtggcaagatcgggacgtctatttgcaaggccctatttgggccaaaatccctcataccgatggccacttccatcctagccctttgatgggcggctttggcttgaagcatcctccgccccaaatcttgattaaaaatacacccgtgcccgccaaccccagcacaacattttccgccgccaaattcgccagtttcattacccaatatagtaccggccaagtgtctgtcgaaattgaatgggaattacaaaaagagaattctaagagatggaaccctgagatccaatataccagtaattataacaaaagcgtgaacgtcgatttcaccgtcgataccaacggggtctacagcgaacccagacctatcgggacacggtatttaaccagaaacttatag

SEQ ID NO: 33 - protein, 598 aa, synthetic construct
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 34 - protein, 533 aa, synthetic construct
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 35 - protein, 294 aa, synthetic construct
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 36 - DNA, 888 bp, synthetic construct
  source 1..888: mol_type = other DNA
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaatgtttcctacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagtaa

SEQ ID NO: 37 - protein, 204 aa, synthetic construct
LETQTQYLTPSLSDSHQQPPLVWELIRWLQAVAHQWQTITRAPTEWVIPREIGIAIPHGWATESSPPAPEPGPCPPTTTTSTNKFPANQEPRTTITTLATAPLGGILTSTDSTATFHHVTGKDSSTTTGDSDPRDSTSSSLTFKSKRSRRMTVRRRLPITLPARFRCLLTRSTSSRTSSARRIKDASRRSQQTSSWCHSMDTSP

SEQ ID NO: 38 - DNA, 738 bp, synthetic construct
  source 1..738: mol_type = other DNA
cgacgtaatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatcc

SEQ ID NO: 39 - protein, 1235 aa, synthetic construct
MFSGGGGPLSPGGKSAARAASGFFAPAGPRGASRGPPPCLRQNFYNPYLAPVGTQQKPTGPTQRHTYYSECDEFRFIAPRVLDEDAPPEKRAGVHDGHLKRAPKVYCGGDERDVLRVGSGGFWPRRSRLWGGVDHAPAGFNPTVTVFHVYDILENVEHAYGMRAAQFHARFMDAITPTGTVITLLGLTPEGHRVAVHVYGTRQYFYMNKEEVDRHLQCRAPRDLCERMAAALRESPGASFRGISADHFEAEVVERTDVYYYETRPALFYRVYVRSGRVLSYLCDNFCPAIKKYEGGVDATTRFILDNPGFVTFGWYRLKPGRNNTLAQPRAPMAFGTSSDVEFNCTADNLAIEGGMSDLPAYKLMCFDIECKAGGEDELAFPVAGHPEDLVIQISCLLYDLSTTALEHVLLFSLGSCDLPESHLNELAARGLPTPVVLEFDSEFEMLLAFMTLVKQYGPEFVTGYNIINFDWPFLLAKLTDIYKVPLDGYGRMNGRGVFRVWDIGQSHFQKRSKIKVNGMVNIDMYGIITDKIKLSSYKLNAVAEAVLKDKKKDLSYRDIPAYYAAGPAQRGVIGEYCIQDSLLVGQLFFKFLPHLELSAVARLAGINITRTIYDGQQIRVFTCLLRLADQKGFILPDTQGRFRGAGGEAPKRPAAAREDEERPEEEGEDEDEREEGGGEREPEGARETAGRHVGYQGARVLDPTSGFHVNPVVVFDFASLYPSIIQAHNLCFSTLSLRADAVAHLEAGKDYLEIEVGGRRLFFVKAHVRESLLSILLRDWLAMRKQIRSRIPQSSPEEAVLLDKQQAAIKVVCNSVYGFTGVQHGLLPCLHVAATVTTIGREMLLATREYVHARWAAFEQLLADFPEAADMRAPGPYSMRIIYGDTDSIFVLCRGLTAAGLTAVGDKMASHISRALFLPPIKLECEKTFTKLLLIAKKKYIGVIYGGKMLIKGVDLVRKNNCAFINRTSRALVDLLFYDDTVSGAAAALAERPAEEWLARPLPEGLQAFGAVLVDAHRRITDPERDIQDFVLTAELSRHPRAYTNKRLAHLTVYYKLMARRAQVPSIKDRIPYVIVAQTREVEETVARLAALRELDAAAPGDEPAPPAALPSPAKRPRETPSPADPPGGASKPRKLLVSELAEDPAYAIAHGVALNTDYYFSHLLGAACVTFKALFGNNAKITESLLKRFIPEVWHPPDDVAARLRTAGFGAVGAGATAEETRRMLHRAFDTLA

SEQ ID NO: 40 - protein, 488 aa, synthetic construct
MTDSPGGVAPASPVEDASDASLGQPEEGAPCQVVLQGAELNGILQAFAPLRTSLLDSLLVMGDRGILIHNTIFGEQVFLPLEHSQFSRYRWRGPTAAFLSLVDQKRSLLSVFRANQYPDLRRVELAITGQAPFRTLVQRIWTTTSDGEAVELASETLMKRELTSFVVLVPQGTPDVQLRLTRPQLTKVLNATGADSATPTTFELGVNGKFSVFTTSTCVTFAAREEGVSSSTSTQVQILSNALTKAGQAAANAKTVYGENTHRTFSVVVDDCSMRAVLRRLQVGGGTLKFFLTTPVPSLCVTATGPNAVSAVFLLKPQKICLDWLGHSQGSPSAGSSASRASGSEPTDSQDSASDAVSHGDPEDLDGAARAGEAGALHACPMPSSTTRVTPTTKRGRSGGEDARADTALKKPKTGSPTAPPPADPVPLDTEDDSDAADGTAARPAAPDARSGSRYACYFRDLPTGEASPGAFSAFRGGPQTPYGFGFP

SEQ ID NO: 41 - protein, 882 aa, synthetic construct
MAAAGGERQLDGQKPGPPHLQQPGDRPAVPGRAEAFLNFTSMHGVQPILKRIRELSQQQLDGAQVPHLQWFRDVAALESPAGLPLREFPFAVYLITGNAGSGKSTCVQTINEVLDCVVTGATRIAAQNMYAKLSGAFLSRPINTIFHEFGFRGNHVQAQLGQYPYTLTSNPASLEDLQRRDLTYYWEVILDLTKRALAASGGEELRNEFRALAALERTLGLAEGALTRLAPATHGALPAFTRSNVIVIDEAGLLGRHLLTAVVYCWWMINALYHTPQYAARLRPVLVCVGSPTQTASLESTFEHQKLRCSVRQSENVLTYLICNRTLREYARLSYSWAIFINNKRCVEHEFGNLMKVLEYGLPITEEHMQFVDRFVVPENYITNPANLPGWTRLFSSHKEVSAYMAKLHAYLKVTREGEFVVFTLPVLTFVSVKEFDEYRRLTHQPGLTIEKWLTANASRITNYSQSQDQDAGHMRCEVHSKQQLVVARNDVTYVLNSQIAVTARLRKLVFGFSGTFRAFEAVLRDDSFVKTQGETSVEFAYRFLSRLIFSGLISFYNFLQRPGLDATQRTLAYARMGELTAEILSLRPKSSGVPTQASVMADAGAPGERAFDFKQLGPRDGGPDDFPDDDLDVIFAGLDEQQLDVFYCHYTPGEPETTAAVHTQFALLKRAFLGRFRILQELFGEAFEVAPFSTYVDNVIFRGCEMLTGSPRGGLMSVALQTDNYTLMGYTYARVFAFADELRRRHATANVAELLEEAPLPYVVLRDQHGFMSVVNTNISEFVESIDSTELAMAINADYGISSKLAMTITRSQGLSLDKVAICFTPGNLRLNSAYVAMSRTTSSEFLRMNLNPLRERHERDDVISEHILSALRDPNVVIVY

SEQ ID NO: 42 - protein, 750 aa, synthetic construct
MDTADIVWVEESVSAITLYAVWLPPRAREYFHALVYFVCRNAAGEGRARFAEVSVTATELRDFYGSADVSVQAVVAAARAATTPAASPLEPLENPTLWRALYACVLAALERQTGPVALFAPLRIGSDPRTGLVVKVERASWGPPAAPRAALLVAEANIDIDPMALAARVAEHPDARLAWARLAAIRDTPQCASAASLTVNITTGTALFAREYQTLAFPPIKKEGAFGDLVEVCEVGLRPRGHPQRVTARVLLPRDYDYFVSAGEKFSAPALVALFRQWHTTVHAAPGALAPVFAFLGPEFEVRGGPVPYFAVLGFPGWPTFTVPATAESARDLVRGAAAAYAALLGAWPAVGARVVLPPRAWPGVASAAAGCLLPAVREAVARWHPATKIIQLLDPPAAVGPVWTARFCFPGLRAQLLAALADLGGSGLADPHGRTGLARLDALVVAAPSEPWAGAVLERLVPDTCNACPALRQLLGGVMAAVCLQIEETASSVKFAVCGGDGGAFWGVFNVDPQDADAASGVIEDARRAIETAVGAVLRANGLRLRHPLCLALEGVYTHAVAWSQAGVWFWNSRDNTDHLGGFPLRGPAYTTAAGVVRDTLRRVLGLTTACVPEEDALTARGLMEDACDRLILDAFNKRLDAEYWSVRVSPFEASDPLPPTAFRGGALLDAEHYWRRVVRVCPGGGESVGVPVDLYPRPLVLPPVDCAHHLREILREIELVFTGVLAGVWGEGGKFVYPFDDKMSFLFA

SEQ ID NO: 43 - protein, 1058 aa, synthetic construct
MGQEDGNRGERRAAGTPVEVTALYATDGCVITSSIALLTNSLLGAEPVYIFSYDAYTHDGRADGPTEQDRFEESRALYQASGGLNGDSFRVTFCLLGTEVGGTHQARGRTRPMFVCRFERADDVAALQDALAHGTPLQPDHIAATLDAEATFALHANMILALTVAINNASPRTGRDAAAAQYDQGASLRSLVGRTSLGQRGLTTLYVHHEVRVLAAYRRAYYGSAQSPFWFLSKFGPDEKSLVLTTRYYLLQAQRLGGAGATYDLQAIKDICATYAIPHAPRPDTVSAASLTSFAAITRFCCTSQYARGAAAAGFPLYVERRIAADVRETSALEKFITHDRSCLRVSDREFITYIYLAHFECFSPPRLATHLRAVTTHDPNPAASTEQPSPLGREAVEQFFCHVRAQLNIGEYVKHNVTPRETVLDGDTAKAYLRARTYAPGALTPAPAYCGAVDSATKMMGRLADAEKLLVPRGWPAFAPASPGEDTAGGTPPPQTCGIVKRLLRLAATEQQGPTPPAIAALIRNAAVQTPLPVYRISMVPTGQAFAALAWDDWARITRDARLAEAVVSAEAAAHPDHGALGRRLTDRIRAQGPVMPPGGLDAGGQMYVNRNEIFNGALAITNIILDLDIALKEPVPFRRLHEALGHFRRGALAAVQLLFPAARVDPDAYPCYFFKSACRPGPASVGSGSGLGNDDDGDWFPCYDDAGDEEWAEDPGAMDTSHDPPDDEVAYFDLCHEVGPTAEPRETDSPVCSCTDKIGLRVCMPVPAPYVVHGSLTMRGVARVIQQAVLLDRDFVEAIGSYVKNFLLIDTGVYAHGHSLRLPYFAKIAPDGPACGRLLPVFVIPPACKDVPAFVAAHADPRRFHFHAPPTYLASPREIRVLHSLGGDYVSFFERKASRNALEHFGRRETLTEVLGRYNVQPDAGGTVEGFASELLGRIVACIETHFPEHAGEYQAVSVRRAVSKDDWVLLQLVPVRGTLQQSLSCLRFKHGRASRATARTFVALSVGANNRLCVSLCQQCFAAKCDSNRLHTLFTIDAGTPCSPSVPCSTSQPSS

SEQ ID NO: 44 - protein, 1196 aa, synthetic construct
METKPKTATTIKVPPGPLGYVYARACPSEGIELLALLSARSGDSDVAVAPLVVGLTVESGFEANVAVVVGSRTTGLGGTAVSLKLTPSHYSSSVYVFHGGRHLDPSTQAPNLTRLCERARRHFGFSDYTPRPGDLKHETTGEALCERLGLDPDRALLYLVVTEGFKEAVCINNTFLHLGGSDKVTIGGAEVHRIPVYPLQLFMPDFSRVIAEPFNANHRSIGENFTYPLPFFNRPLNRLLFEAVVGPAAVALRCRNVDAVARAAAHLAFDENHEGAALPADITFTAFEASQGKTPRGGRDGGGKGPAGGFEQRLASVMAGDAALALESIVSMAVFDEPPTDISAWPLFEGQDTAAARANAVGAYLARAAGLVGAMVFSTNSALHLTEVDDAGPADPKDHSKPSFYRFFLVPGTHVAANPQVDREGHVVPGFEGRPTAPLVGGTQEFAGEHLAMLCGFSPALLAKMLFYLERCDGGVIVGRQEMDVFRYVADSNQTDVPCNLCTFDTRHACVHTTLMRLRARHPKFASAARGAIGVFGTMNSMYSDCDVLGNYAAFSALKRADGSETARTIMQETYRAATERVMAELETLQYVDQAVPTAMGRLETIITNREALHTVVNNVRQVVDREVEQLMRNLVEGRNFKFRDGLGEANHAMSLTLDPYACGPCPLLQLLGRRSNLAVYQDLALSQCHGVFAGQSVEGRNFRNQFQPVLRRRVMDMFNNGFLSAKTLTVALSEGAAICAPSLTAGQTAPAESSFEGDVARVTLGFPKELRVKSRVLFAGASANASEAAKARVASLQSAYQKPDKRVDILLGPLGFLLKQFHAAIFPNGKPPGSNQPNPQWFWTALQRNQLPARLLSREDIETIAFIKKFSLDYGAINFINLAPNNVSELAMYYMANQILRYCDHSTYFINTLTAIIAGSRRPPSVQAAAAWSAQGGAGLEAGARALMDAVDAHPGAWTSMFASCNLLRPVMAARPMVVLGLSISKYYGMAGNDRVFQAGNWASLMGGKNACPLLIFDRTRKFVLACPRAGFVCAASSLGGGAHESSLCEQLRGIISEGGAAVASSVFVATVKSLGPRTQQLQIEDWLALLEDEYLSEEMMELTARALERGNGEWSTDAALEVAHEAEALVSQLGNAGEVFNFGDFGCEDDNATPFGGPGAPGPAFAGRKRAFHGDDPFGEGPPDKKGDLTLDML

SEQ ID NO: 45 - DNA, 578 bp, synthetic construct
  source 1..578: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatg

SEQ ID NO: 46 - DNA, 571 bp, synthetic construct
  source 1..571: mol_type = other DNA
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatagttatc

SEQ ID NO: 47 - protein, 19 aa, synthetic construct
ATNFSLLKQAGDVEENPGP

SEQ ID NO: 48 - protein, 18 aa, synthetic construct
EGRGSLLTCGDVEENPGP

SEQ ID NO: 49 - protein, 119 aa, synthetic construct
LAHHHQSPQSGIRTTAGVLCFLGTSTSDPSTDSTRESRSTRQTPRPSSTTKPTTGSSTAETTRTSSTTTPTRSFRSALKKIRLLGATSDEQSSRRKRGFLNLWAWLRNLLRRLREKRGR

SEQ ID NO: 50 - protein, 626 aa, synthetic construct
MESTVGPACPPGRTVTKRPWALAEDTPRGPDSPPKRPRPNSLPLTTTFRPLPPPPQTTSAVDPSSHSPVNPPRDQHATDTADEKPRAASPALSDASGPPTPDIPLSPGGTHARDPDADPDSPDLDSMWSASVIPNALPSHILAETFERHLRGLLRGVRAPLAIGPLWARLDYLCSLAVVLEEAGMVDRGLGRHLWRLTRRGPPAAADAVAPRPLMGFYEAATQNQADCQLWALLRRGLTTASTLRWGPQGPCFSPQWLKHNASLRPDVQSSAVMFGRVNEPTARSLLFRYCVGRADDGGEAGADTRRFIFHEPSDLAEENVHTCGVLMDGHTGMVGASLDILVCPRDIHGYLAPVPKTPLAFYEVKCRAKYAFDPMDPSDPTASAYEDLMAHRSPEAFRAFIRSIPKPSVRYFAPGRVPGPEEALVTQDQAWSEAHASGEKRRCSAADRALVELNSGVVSEVLLFGAPDLGRHTISPVSWSSGDLVRREPVFANPRHPNFKQILVQGYVLDSHFPDCPPHPHLVTFIGRHRTSAEEGVTFRLEDGAGALGAAGPSKASILPNQAVPIALIITPVRIDPEIYKAIQRSSRLAFDDTLAELWASRSPGPGPAAAETTSSSPTTGRSSR

SEQ ID NO: 51 - protein, 775 aa, synthetic construct
MEPRPGASTRRPEGRPQREPAPDVWVFPCDRDLPDSSDSEAETEVGGRGDADHHDDDSASEADSTDTELFETGLLGPQGVDGGAVSGGSPPREEDPGSCGGAPPREDGGSDEGDVCAVCTDEIAPHLRCDTFPCMHRFCIPCMKTWMQLRNTCPLCNAKLVYLIVGVTPSGSFSTIPIVNDPQTRMEAEEAVRAGTAVDFIWTGNQRFAPRYLTLGGHTVRALSPTHPEPTTDEDDDDLDDADYVPPAPRRTPRAPPRRGAAAPPVTGGASHAAPQPAAARTAPPSAPIGPHGSSNTNTTTNSSGGGGSRQSRAAAPRGASGPSGGVGVGVGVVEAEAGRPRGRTGPLVNRPAPLANNRDPIVISDSPPASPHRPPAAPMPGSAPRPGPPASAAASGPARPRAAVAPCVRAPPPGPGPRAPAPGAEPAARPADARRVPQSHSSLAQAANQEQSLCRARATVARGSGGPGVEGGHGPSRGAAPSGAAPLPSAASVEQEAAVRPRKRRGSGQENPSPQSTRPPLAPAGAKRAATHPPSDSGPGGRGQGGPGTPLTSSAASASSSSASSSSAPTPAGAASSAAGAASSSASASSGGAVGALGGRQEETSLGPRAASGPRGPRKCARKTRHAETSGAVPAGGLTRYLPISGVSSVVALSPYVNKTITGDCLPILDMETGNIGAYVVLVDQTGNMATRLRAAVPGWSRRTLLPETAGNHVMPPEYPTAPASEWNSLWMTPVGNMLFDQGTLVGALDFRSLRSRHPWSGEQGASTRDEGKQ

SEQ ID NO: 52 - protein, 1298 aa, synthetic construct
MASENKQRPGSPGPTDGPPPTPSPDRDERGALGWGAETEEGGDDPDHDPDHPHDLDDARRDGRAPAAGTDAGEDAGDAVSPRQLALLASMVEEAVRTIPTPDPAASPPRTPAFRADDDDGDEYDDAADAAGDRAPARGREREAPLRGAYPDPTDRLSPRPPAQPPRRRRHGRWRPSASSTSSDSGSSSSSSASSSSSSSDEDEDDDGNDAADHAREARAVGRGPSSAAPAAPGRTPPPPGPPPLSEAAPKPRAAARTPAASAGRIERRRARAAVAGRDATGRFTAGQPRRVELDADATSGAFYARYRDGYVSGEPWPGAGPPPPGRVLYGGLGDSRPGLWGAPEAEEARRRFEASGAPAAVWAPELGDAAQQYALITRLLYTPDAEAMGWLQNPRVVPGDVALDQACFRISGAARNSSSFITGSVARAVPHLGYAMAAGRFGWGLAHAAAAVAMSRRYDRAQKGFLLTSLRRAYAPLLARENAALTGAAGSPGAGADDEGVAAVAAAAPGERAVPAGYGAAGILAALGRLSAAPASPAGGDDPDAARHADADDDAGRRAQAGRVAVECLAACRGILEALAEGFDGDLAAVPGLAGARPASPPRPEGPAGPASPPPPHADAPRLRAWLRELRFVRDALVLMRLRGDLRVAGGSEAAVAAVRAVSLVAGALGPALPRDPRLPSSAAAAAADLLFDNQSLRPLLAAAASAPDAADALAAAAASAAPREGRKRKSPGPARPPGGGGPRPPKTKKSGADAPGSDARAPLPAPAPPSTPPGPEPAPAQPAAPRAAAAQARPRPVAVSRRPAEGPDPLGGWRRQPPGPSHTAAPAAAALEAYCSPRAVAELTDHPLFPVPWRPALMFDPRALASIAARCAGPAPAAQAACGGGDDDDNPHPHGAAGGRLFGPLRASGPLRRMAAWMRQIPDPEDVRVVVLYSPLPGEDLAGGGASGGPPEWSAERGGLSCLLAALANRLCGPDTAAWAGNWTGAPDVSALGAQGVLLLSTRDLAFAGAVEFLGLLASAGDRRLIVVNTVRACDWPADGPAVSRQHAYLACELLPAVQCAVRWPAARDLRRTVLASGRVFGPGVFARVEAAHARLYPDAPPLRLCRGGNVRYRVRTRFGPDTPVPMSPREYRRAVLPALDGRAAASGTTDAMAPGAPDFCEEEAHSHAACARWGLGAPLRPVYVALGREAVRAGPARWRGPRRDFCARALLEPDDDAPPLVLRGDDDGPGALPPAPPGIRWASATGRSGTVLAAAGAVEVLGAEAGLATPPRREVVDWEGAWDEDDGGAFEGDGVL

SEQ ID NO: 53 - protein, 420 aa, synthetic construct
MADISPGAFAPCVKARRPALRSPPLGTRKRKRPSRPLSSESEVESDTALESEVESETASDSTESGDQDEAPRIGGRRAPRRLGGRFFLDMSAESTTGTETDASVSDDPDDTSDWSYDDIPPRPKRARVNLRLTSSPDRRDGVIFPKMGRVRSTRETQPRAPTPSAPSPNAMLRRSVRQAQRRSSARWTPDLGYMRQCINQLFRVLRVARDPHGSANRLRHLIRDCYLMGYCRARLAPRTWCRLLQVSGGTWGMHLRNTIREVEARFDATAEPVCKLPCLETRRYGPECDLSNLEIHLSATSDDEISDATDLEAAGSDHTLASQSDTEDAPSPVTLETPEPRGSLAVRLEDEFGEFDWTPQEGSQPWLSAVVADTSSVERPGPSDSGAGRAAEDRKCLDGCRKMRFSTACPYPCSDTFLRP